(19) United States
(12) Patent Application Publication
(10) Pub. No.: US 2026/0115315 A1
(43) Pub. Date: Apr. 30, 2026

(54) DRUG DELIVERY SYSTEM FOR BLOOD-BRAIN BARRIER PENETRATION

(71) Applicant: KOREA INSTITUTE OF SCIENCE AND TECHNOLOGY, Seoul (KR)

(72) Inventors: Dae-Ro AHN, Seoul (KR); Kyoung-Ran KIM, Seoul (KR); Ji hyun BACK, Seoul (KR); Ji Eun LEE, Seoul (KR); Young Tag KO, Gimpo-si (KR); Ji Hee KANG, Incheon (KR)

(73) Assignee: KOREA INSTITUTE OF SCIENCE AND TECHNOLOGY, Seoul (KR)

(21) Appl. No.: 19/263,946

(22) Filed: Jul. 9, 2025

(30) Foreign Application Priority Data
Jul. 10, 2024 (KR) 10-2024-0091296

(51) Int. Cl. A61K 47/69; A61K 31/713; A61K 47/54; A61P 35/00; C12N 15/113

(52) U.S. Cl. CPC A61K 47/6931; A61K 31/713; A61K 47/549; A61P 35/00; C12N 15/113; C12N 2310/11; C12N 2310/14

(57) ABSTRACT

This invention relates to a drug delivery vehicle for penetrating the Blood-Brain Barrier (BBB). Said drug delivery vehicle is a cube-structured nanostructure formed from a double-stranded deoxyribonucleic acid (DNA) framework, specifically D-form DNA. This drug delivery vehicle forms a protein corona on its surface within the serum, and through this, it passes the BBB via receptor-mediated transcytosis. The present invention provides a drug delivery vehicle for the purpose of being loaded with drugs, such as antisense oligonucleotides (ASOs), to deliver them to brain tissue for the treatment of brain tumors like glioblastoma.

### CROSS-REFERENCE TO RELATED APPLICATION

This application claims the benefit of Korean Patent Application No. 10-2024-0091296, filed on Jul. 10, 2024, at the Korean Intellectual Property Office, the entire disclosure of which is incorporated herein by reference for all purposes.

### REFERENCE TO AN ELECTRONIC SEQUENCE LISTING

The contents of the electronic sequence listing (“NewApp_0210520010_20250704LFC_APC-2023-0696.xml”; Size is 28,745 bytes and it was created on Jul. 4, 2025) is herein incorporated by reference in its entirety.

### BACKGROUND

### 1. Field of the Invention

One or more embodiments relate to the use of a cube-shaped DNA nanostructure as a drug delivery system for the purpose of penetrating the blood-brain barrier (BBB).

### 2. Description of Related Art

Oligonucleotide therapeutics (OTs) such as small interference RNAs (siRNAs) and antisense oligonucleotides (ASOs) hybridize with disease-related mRNA sequences and downregulate the expression of the downstream proteins. OTs have emerged as a strategy for treating diseases that are difficult to treat with traditional small molecule drugs. With systemic administration, most of the clinically available OTs may be delivered to the liver by lipid nanoparticles (LNPs) or GalNacs (triantennary or N-acetylgalactosamine), the hepatocyte-specific ligands which are used to treat various liver diseases. However, a suitable method for systemic administration and delivery of OTs into organs or tissues other than the liver has not yet been developed.

The blood-brain barrier (BBB) as a protective layer of the central nervous system (CNS) presents a significant challenge for the delivery of systemically injected therapeutics to the brain. The BBB is composed of brain microvascular endothelial cells (BMEC), which are components of brain capillaries, and is composed of other brain tissue components such as pericytes, smooth muscle cells, and astrocytes. Only a limited number of hydrophobic substances with a low molecular weight of <500 Da and fewer than 10 hydrogen bonds may passively diffuse through the BBB. While the highly ionic and hydrophilic small molecules unable to diffuse through the BBB may reach the brain parenchyma via the paracellular pathway, the tight junctions between endothelial cells filled with multiprotein junctional complexes maintain the low paracellular permeability of the BBB. Nutrients and proteins required for brain function and homeostasis may pass through the BBB by transcytosis mediated by specific transporters and receptors expressed on each BMEC. Another transcytosis pathway for BBB penetration is adsorptive-mediated transcytosis, which is triggered by non-specific interactions between positively charged molecules and the negatively charged BMEC membrane. Polyanionic macromolecules such as OTs are intrinsically unable to penetrate the BBB through diffusion or transcytosis. OTs may be delivered into the brain parenchyma via BBB-bypassing routes such as intraventricular and intrathecal injections. However, these invasive methods may limit the applications for OTs. If BBB penetration via a systemic route, such as intravenous administration, is available, OTs may be delivered to the brain parenchyma in a relatively less invasive manner.

A prevalent approach for enhancing the delivery of OTs to the brain via a systemic route involves the conjugation of OT-loaded nanocarriers with ligands that target endocytic receptors in BMEC and promote transcytosis through the BBB. Various receptor-targeting ligands have been conjugated with nanocarriers to improve the distribution of OTs within the brain. However, this delivery method does not always guarantee an increase in brain distribution, and in certain cases, there is no considerable improvement in brain distribution or even a decrease in degree of brain delivery compared to when unconjugated nanocarriers are used. It is suspected that the formation of a protein corona, a layer of serum proteins adsorbed onto the surface of the nanocarriers, inhibits the ligand-based enhancement of brain delivery. Meanwhile, the transcytosis efficiency may also be limited in case where receptors are saturated with high levels of endogenous ligands and/or the targeting ligands are not sufficiently stable. Given that the protein corona inevitably forms in situ and affects the in vivo distribution of nanocarriers, the design of nanocarriers for enhanced systemic delivery of OTs to the brain is important.

### SUMMARY

The inventors discovered a DNA construct with a protein corona that may enhance brain distribution through in vivo screening of various DNA nanostructures and created the present disclosure. More specifically, the inventors prepared six different wireframe DNA nanostructures and examined their biodistribution upon intravenous injection (FIG. 1). The results showed that a cube-shaped DNA nanostructure had brain distribution properties. The inventors subsequently investigated the role of the protein corona in the brain distribution of this DNA nanostructure. Finally, the inventors employed the DNA nanostructure as a carrier for delivering ASOs to treat glioblastoma multiforme (GBM) in an orthotopic GBM mouse model. The results showed that the cube-shaped DNA nanostructure had practical potential as a platform for enhanced systemic delivery of OTs to the brain.

Hence, the technical goal to be achieved by the present disclosure is to provide a DNA nanostructure as a drug delivery system for penetrating the BBB.

However, the technical goal to be achieved is not limited to the one described above, and other goals not mentioned above will be clearly understood by one of ordinary skill in the art from the following description.

To achieve the technical goal described above, the present disclosure provides a DNA nanostructure as a drug delivery system for penetrating the blood-brain barrier (BBB).

The inventors fabricated DNA nanostructures in the structures of a tetrahedron, a triangular prism (the term triangular prism is used interchangeably with the term trigonal prism), and a regular hexahedron and confirmed the BBB permeability of the nanostructures. As a result, it was confirmed that the regular hexahedron structure has excellent BBB permeability. Therefore, the DNA nanostructure of the present disclosure is a cube structure using double-stranded deoxyribonucleic acids (DNA) as a frame.

In the present disclosure, the DNA nanostructure is a cube structure. As used herein, the term cube structure is used interchangeably with the term regular hexahedral structure, but although the intention is that the terms may refer to a regular hexahedron, the terms may not strictly refer to a regular hexahedron.

When one side of the cube-structured DNA nanostructure of the present disclosure is 5 bp or less in length, there is a problem with the stability of the structure, and when it exceeds 30 bp in length, the BBB permeation efficiency is reduced. Thus, one side of the DNA nanostructure of the present disclosure may preferably have a length of greater than 5 bp and less than 30 bp, and may have a length of greater than 5 bp and less than 25 bp, greater than 7 bp and less than 25 bp, greater than 9 bp and less than 25 bp, greater than 5 bp and less than 20 bp, greater than 7 bp and less than 20 bp, greater than 9 bp and less than 20 bp, greater than 5 bp and less than 17 bp, greater than 7 bp and less than 17 bp, greater than 9 bp and less than 17 bp, greater than 18 bp and less than 25 bp, greater than 18 bp and less than 25 bp, greater than 18 bp and less than 25 bp.

As used herein, the term double-stranded DNA is used interchangeably with the terms skeleton and frame in that double-stranded DNA forms the frame of a DNA nanostructure, and the term frame is used interchangeably with the term wireframe in that the frame is double-stranded.

Meanwhile, the inventors found that the cube-shaped DNA nanostructures formed from D-form DNA had better BBB permeability than the cube-shaped DNA nanostructures formed from L-form DNA. Therefore, the DNA nanostructure of the present disclosure may consist of D-form DNA.

As used herein, a DNA nanostructure is represented by its structure and the type of DNA constituting the nanostructure. For example, a cube-shaped DNA nanostructure consisting of D-form DNA is denoted as D-Cb.

The DNA nanostructure of the present disclosure may penetrate the BBB and further show accumulation in brain tumor cells and thus, may be used as a drug delivery system for treating brain tumors by loading a drug capable of inhibiting the proliferation of tumor cells or inducing tumor cell death.

Specifically, the inventors loaded ASO targeting PLK1 mRNA, a target molecule for the treatment of glioblastoma, into D-Cb to treat glioblastoma cells (U87MG) in order to evaluate the gene silencing effect. As a result, it was confirmed that D-Cb significantly enhanced the cellular uptake of ASO, and the cellularly introduced ASO inhibited the expression of the target molecule, i.e. PLK1.

In addition, the inventors loaded ASO into D-Cb and intravenously injected it into GBM-induced mice. Treatment with ASO@D-Cb resulted in a higher distribution level in the brain than treatment with ASO alone. The D-Cb protects the ASO until the ASO enters the target cells. Furthermore, ASO loaded onto D-Cb was introduced into glioblastoma cells and exhibited therapeutic efficacy.

Accordingly, the present disclosure provides a pharmaceutical composition for treating brain tumors including the drug-loaded DNA nanostructure as an active ingredient.

In one embodiment of the present disclosure, the drug may be a known drug that inhibits the proliferation of cancer cells or induces cell death, and preferably may be an oligonucleotide or a hydrophobic anticancer drug such as doxorubicin.

According to embodiments, the present disclosure provides a DNA nanostructure for penetrating the BBB. It has been confirmed that the DNA nanostructure of the present disclosure has high stability in vivo, efficiently penetrates the BBB, has brain tissue selectivity, and may be accumulated in brain tumors. In addition, the DNA nanostructure of the present disclosure may deliver drugs to brain tissue stably without being affected by brain accessibility even after drug loading and thus, may be used as a drug delivery system capable of loading drugs for treating brain tumors. Further, it is expected that the DNA nanostructure of the present disclosure may be used in the development of treatments for degenerative brain diseases, whose prevalence is increasing since the population is aging.

### BRIEF DESCRIPTION OF THE DRAWINGS

These and/or other aspects, features, and advantages of the invention will become apparent and more readily appreciated from the following description of embodiments, taken in conjunction with the accompanying drawings.

FIG. 1 is a schematic diagram of the process whereby a protein corona is formed on the DNA cube following intravenous (IV) injection, and receptor-mediated transcytosis for BBB penetration occurs.

FIGS. 2A to 2E show the design and characterization results of self-assembled wireframe DNA nanostructures:

FIG. 2A: Schematic structures of six types of DNA nanostructures.

FIG. 2B: AFM analysis of DNA nanostructures. Schematic diagrams of the DNA nanostructure shown in the inset images are? presented beside the AFM images. Scale bar: 10 nm.

FIG. 2C: Hydrodynamic sizes of DNA nanostructures estimated on DLS (n=3, mean±SEM, SEM represents the standard error of the mean).

FIG. 2D: Agarose gel (1%) electrophoresis results of DNA nanostructures after incubation in 50% mouse serum. C represents the control group, which is the structure when serum is absent. S represents serum only. Asterisks and arrows indicate bands of serum and undamaged structures, respectively.

FIG. 2E: 10% non-denaturing PAGE results showing the assembly of DNA nanostructures. M represents the 100 bp size marker.

FIGS. 3A to 3C show the results of the in vivo biodistribution of DNA nanostructures over time in BALB/c mice after intravenous injection of Cy5.5-labeled structures (2 μM, 200 μL).

FIG. 4 is ex vivo images of major organs showing the distribution of Cy5.5-labeled DNA nanostructures at 2 hours post injection (2 μM, 200 μL) in BALB/c mice.

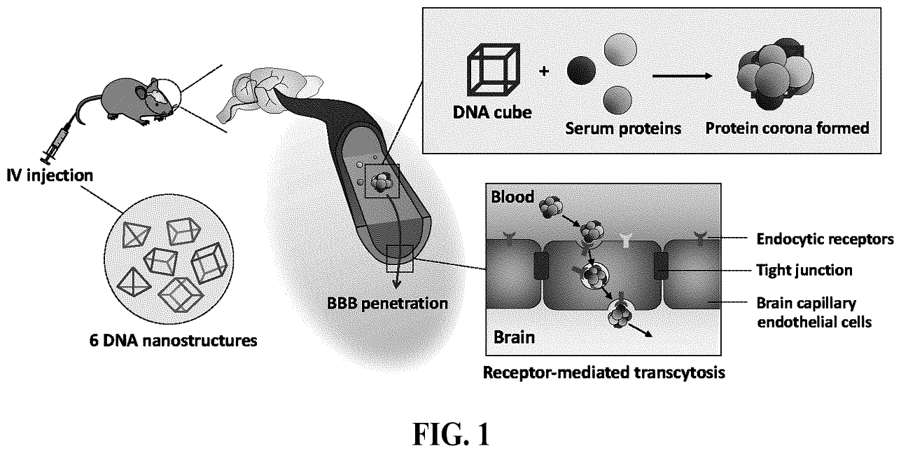

FIG. 5 shows the results of distributing DNA nanostructures in a brain:
(a) Ex vivo images of a mouse brain collected 2 hours after intravenous injection of DNA nanostructures.
(b) Distribution levels of intact D-Cb and L-Cb in the brain (% ID/g) estimated by total Cy5.5 intensity of homogenized brain tissue lysates (Total, Cy5.5). The band intensities of fluorescently labeled oligonucleotides by PAGE analysis of brain tissue lysates were quantified to calculate the delivery amount of intact D-Cb and L-Cb (n=3, mean±SEM, SEM represents the standard error of the mean, ns; not statistically significant).
(c) Schematic diagram of an in vitro BBB monolayer model.
(d) Transcytosis efficiency of DNA nanostructures and single-stranded (SS) D-DNA (D-Cb-S1) across the BBB model at different time points (n=4, mean±SEM, ****P<0.0001 vs. SS, D-Tp, D-Td, and L-Cb).

FIGS. 6A and 6B show the results of the intact form analysis of an S1 oligonucleotide in brain lysate 2 hours after injection (n=3). The amount estimated by quantification of the band intensity is shown in FIG. 5B. M represents the size marker. FIG. 6A shows the result of the intact form analysis of an S1 oligonucleotide labeled with FAM, and FIG. 6B shows the result of the intact form analysis of an S1 oligonucleotide labeled with Cy5.5.

FIG. 7 is a representative ESI-MS spectrum (n=2) for detecting an intact biotinylated S5 oligonucleotide in brain lysate.

In FIG. 8, (a) shows a fluorescence image of FAM-labeled DNA nanostructures and single-stranded D-DNA (SS, D-Cb-S1) after having passed through an in vitro BBB model (n=4), (b) shows the TEER measurement results before and after transcytosis (n=4, mean±SEM), and (c) shows the apparent permeability coefficient (Pₐₚₚ) of DNA nanostructures and SS measured using an in vitro BBB model (n=4, mean±SEM, ****P<0.0001 vs. D-Cb).

FIG. 9 shows the results of identifying the effect of a protein corona on D-Cb for brain delivery:
(a) Schematic presentation of the preparation of a protein corona by incubating biotinylated D-Cb and L-Cb immobilized on streptavidin-coated-magnetic beads in mouse serum.
(b) SDS-PAGE (12%) analysis of proteins extracted from mouse serum with D-Cb and L-Cb. The protein bands were stained by Coomassie Blue. M represents the size marker.
(c) Venn diagram showing the number of proteins identified from three replicates of LC-MS/MS runs for each group of samples.
(d) Volcano plot of 210 proteins identified among the proteins pulled-down with D-Cb and L-Cb. The proteins shown above the gray horizontal line are the 165 proteins that were statistically significant (P-value <0.05). Orange spots represent 15 protein ligands of: selenoprotein P (SELENOP), vitamin D-binding protein (GC), -2-glycoprotein 1 (APOH), plasminogen (PLG), gelsolin (GSN), transthyretin (TTR), hemoglobin subunit β-2 (HBB-B2), β-2-microglobulin (B2M), retinol-binding protein 4 (RBP4), apolipoprotein M (APOM), cathepsin B (CTSB), clusterin (CLU), serotransferrin (TF), and apolipoprotein E (APOE).
(e) Heat map showing hierarchical clustering of the 15 proteins with statistically significant changes (P-value <0.05) between the two types of samples extracted with D-Cb and L-Cb. The rows represent each protein and the columns show three LC-MS/MS runs of samples extracted with beads, L-Cb, and D-Cb. Hierarchical clustering of the 15 proteins was performed using Perseus software (1.6.14.0) based on log-transformed normalized abundance values after the data was z-score normalized.
(f) Flow cytometric analysis of bEnd.3 cells after treatment with D-Cb in 10% FBS in the presence or absence of anti-TfR, -LRP1, -LRP2, or -LDLR antibodies (n=3, mean±SEM, ns; not statistically significant, *p<0.05. ***p<0.001, ****p<0.0001 vs No Ab groups). Ab represents antibodies.
(g) Transcytosis efficiency of D-Cb in 10% FBS containing medium in the presence or absence of antibodies (n=4, mean±SEM) **P<0.01 versus LRP1 Ab, ****P<0.0001 versus TfR Ab, LRP2 Ab, and LDLR Ab).

FIG. 10 is a heat map showing hierarchical clustering of the 165 proteins with statistically significant changes (P-value <0.05) in the three types of samples extracted with beads, D-Cb, and L-Cb. The rows represent each protein and the columns show technical replicates of samples extracted with beads, D-Cb and L-Cb. Hierarchical clustering of the 165 proteins was performed using Perseus software (1.6.14.0) based on log-transformed normalized abundance values after the data was z-score normalized.

FIG. 11 is a heat map showing hierarchical clustering of the 133 proteins with statistically significant changes (P-value <0.05) between the two types of samples extracted with D-Cb and L-Cb. The rows represent each protein and the columns show three LC-MS/MS runs of samples extracted with beads, D-Cb and L-Cb. Hierarchical clustering of the 133 proteins was performed using Perseus software (1.6.14.0) based on log-transformed normalized abundance values after the data was z-score normalized.

In FIG. 12, (a) shows a fluorescence image of FAM-labeled D-Cb (initial dose: 1 μM, 100 μL) after having passed through a BBB monolayer in the presence or absence of antibodies (TfR Ab, LRP1 Ab, LRP2 Ab, or LDLR Ab), and (b) shows the result of quantifying the fluorescence of FAM-labeled D-Cb after having passed through a BBB monolayer in the presence or absence of anti-TfR, anti-LRP1, anti-LRP2, or anti-LDLR antibodies (n=3, mean±SEM, ns; no statistically significant vs. no Ab groups). Ab represents antibodies. (c) is the TEER result before and after antibody treatment (n=4, means±SEM).

FIG. 13 shows the results of analysis of the protein corona-assisted distribution of D-Cb in glioblastoma tumors:
(a) Schematic diagram showing the protein corona-assisted distribution of D-Cb in glioblastoma tumors.
(b) Ex vivo images of brains collected 2 hours after intravenous injection of Cy5.5-labeled DNA nanostructures in glioblastoma mice. Tumor areas were identified within brain tissue using Luciferase bioluminescence imaging.
(c) Distribution levels of D-Cb and L-Cb in a brain estimated by total Cy5.5 intensity (F.I.) (Total, Cy5.5) of homogenized brain lysates (n=3, mean±SEM, SEM represents the standard error of the mean, ns; not statistically significant).
(d) Representative fluorescence images of brain tissue sections. Green: tumor (U87MG-FLuc-GFP), blue: nuclei, red: D-Cb or L-Cb, scale bar: 25 μm.
(e) Flow cytometric analysis of bEnd.3 cells after treatment with D-Cb in 10% FBS in the presence or absence of anti-TfR, -LRP1, -LRP2, or -LDLR antibodies (n=3, mean±SEM, ns; not statistically significant, *p<0.05. ***p<0.001, ****p<0.0001 vs No Ab groups). Ab represents antibodies.
(f) Schematic diagram of an in vitro BTB monolayer model.
(g) Fluorescence microscopic images showing the uptake of FAM-labeled D-Cb or L-Cb (green) into U87MG cells after transcytosis through the endothelial cell layer in an in vitro BTB model. The nuclei were stained with DAPI (blue). Scale bar: 50 μm.
(h) Relative FAM fluorescence intensity (F.I.) of U87MG cells in (g) (n=5, mean±SEM, **P<0.01).

FIG. 14 shows the PAGE analysis results for the intact form of an S1 oligonucleotide in brain lysates (GBM mouse) 2 hours after injection (a; Cy5.5-labeled D-Cb and b; Cy5.5-labeled L-Cb).

FIG. 15 shows flow cytometric analysis of U87MG cells after treatment with L-Cb in 10% FBS containing medium in the presence or absence of anti-TfR, anti-LRP1, anti-LRP2, or anti-LDLR antibodies (n=3, mean±SEM, ns; not statistically significant vs. no Ab groups).

FIGS. 16A to 16I show the results of brain delivery experiments involving delivery of PLK1 ASO using D-Cb:

FIG. 16A shows a schematic diagram of D-Cb loaded with ASO targeting PLK1 (ASO@D-Cb).

FIG. 16B shows confocal microscopic images of U87MG cells after treatment with PBS, ASO, and ASO@D-Cb. Magnification 400×, scale bar: 25 μm, blue: nuclei, red: ASO.

FIG. 16C shows the cellular uptake levels of ASO and ASO@D-Cb in U87MG cells (ns; not statistically significant, ****P<0.0001 vs. PBS group).

FIG. 16D shows the relative PLK1 mRNA and protein levels in U87MG cells after ASO@D-Cb treatment, which were analyzed by qRT-PCR and western blotting, respectively. LF represents lipofectamine RNAiMax (n=3, mean±SEM, ns; not statistically significant, ****P<0.0001 vs. PBS treatment group).

FIG. 16E shows the time-dependent brain distribution of ASO or ASO@D-Cb in glioblastoma mice (U87MG-FLuc-GFP orthotopic mouse model).

FIG. 16F shows ex vivo images of a brain displaying the distribution of ASO and ASO@D-Cb at 2 hours or 24 hours post-injection (2 μM, 200 μL).

FIG. 16G shows the brain distribution levels (% ID/g) of ASO or ASO@D-Cb as estimated by the total Cy5.5 intensity of homogenized brain lysates from healthy or GBM mice (total) and the band intensity of Cy5.5-labeled intact oligonucleotides of the lysates (intact) from PAGE results (n=3, mean±SEM). ND indicates not detected.

FIG. 16H shows representative fluorescence images of brain tissue sections illustrating the brain penetration of ASO@D-Cb. Green: tumor (U87MG-FLuc-GFP), blue: nuclei, red: ASO or ASO@D-Cb, magnification: 200×, scale bar: 100 μm.

FIG. 16I shows the relative distribution levels of ASO and ASO@D-Cb in tumor and normal regions, estimated by image analysis of the sectioned brain tissues using ImageJ (n=5, mean±SEM).

FIG. 17 shows the PAGE (6%) results for verifying the stepwise self-assembly of ASO-loaded D-Cb.

FIG. 18 shows the DLS results showing the hydrodynamic sizes of D-Cb and ASO@D-Cb (mean±SEM, n=3).

FIG. 19 shows the results of Western blotting confirming the level of PLK1 in U87MG cells after each sample treatment (n=3). LF represents lipofectamine RNAiMax (used as a positive control).

FIG. 20 shows ex vivo images of major organs resected from glioblastoma mice at 2 hours and 24 hours after administration of Cy5.5-labeled ASO and ASO@D-Cb in the glioblastoma-induced mice (brain: B, heart: H, lung: Lu, liver: Lv, kidney: K, spleen: S).

FIG. 21 shows the results of intact form analysis of Cy5.5-labeled ASO (FIG. 21A) or ASO-conjugated S6 oligonucleotide (FIG. 21B) in brain lysates at 2 hours after injection (GBM mice, n=3). The graph on the right shows the calibration curve of band intensity for quantifying the intact form of the oligonucleotides. The estimated amount is shown in FIG. 16G. M represents the size marker.

FIG. 22 shows ex vivo images of (a) major organs or (b) brain resected from healthy mice at 2 hours after administration of Cy5.5-labeled ASO and ASO@D-Cb in the healthy mice (n=3, brain: B, heart: H, lung: Lu, liver: Lv, kidney: K, spleen: S).

FIGS. 23A and 23B show the results of intact form analysis of Cy5.5-labeled ASO (FIG. 23A) or ASO-conjugated S6 oligonucleotide (FIG. 23B) in brain lysates at 2 hours after injection (healthy mice, n=3). The graph on the right shows the standard curve of band intensity for quantifying the intact form of the oligonucleotides. FIG. 16G shows the estimated amount. M represents the size marker.

FIGS. 24A to 24F show the results confirming the therapeutic efficacy of ASO@D-Cb for treating glioblastoma in vivo.

FIG. 24A: Schematic diagram of intravenous injection of ASO@D-Cb into U87MG tumor-bearing mice to treat glioblastoma.

FIG. 24B: Schematic diagram of the ASO treatment experiment for downregulation of PLK1 in a GBM mouse model.

FIG. 24C: Luminescence images of BALB/c nude mice bearing orthotopic U87MG-FLuc-GFP tumors after treatment with PBS, free ASO, ASO-SC@D-Cb, or ASO@D-Cb. The mice were intravenously injected at a dose of 400 pmol ASO (20 nmol/kg) per mouse on days 14, 16, 18, 20, and 22 after tumor implantation.

FIG. 24D: Luminescence levels of mice estimated in the IVIS system (n=4, mean±SEM, **P<0.01).

FIG. 24E: Histological images of H&E stained brain tissue sections from PBS, ASO, ASO-SC@D-Cb, or ASO@D-Cb treated mice. The tumor regions of interest in the brain sections, indicated by black boxes, were magnified. Scale bars of the magnified images indicate 100 μm. The red dotted lines indicate the tumor region, and the yellow arrows indicate chromatin shrinkage due to nuclear damage. Apoptotic tumor cells (green) in brain sections were analyzed by TUNEL assay (magnification: 200×, scale bar: 100 μm).

FIG. 24F: The analysis results of PLK1 mRNA levels using qRT-PCR in GBM mice treated with ASO, and PLK1 protein levels using Western blotting in brain tissue lysates (n=4, mean±SEM, ****P<0.0001 vs. PBS treatment group).

FIG. 25 shows H&E staining images of heart, liver, lung, kidney and spleen tissues to confirm the systemic toxicity forPBS, ASO, ASO-SC@D-Cb andASO@D-Cb (magnification: 200×, scale bar: 100 μm).

FIG. 26 is a western blot image of PLK1 protein in the brains of glioblastoma mice after treatment with PBS, ASO, ASO-SC@D-Cb, or ASO@D-Cb (n=4).

FIG. 27 shows the results of monitoring mouse body weight every two days during ASO@D-Cb treatment. Data is expressed as mean±SEM (n=4) (****P<0.0001).

### DETAILED DESCRIPTION

Three-dimensional DNA nanostructures are known to be usable as a drug delivery system, but DNA nanostructures capable of penetrating the BBB have not yet been developed. The BBB is considered to be the primary barrier to drug delivery into the brain. The inventors developed DNA nanostructures capable of penetrating the BBB in order to deliver drugs into the brain.

More specifically, the inventors attempted to determine whether the structure of a three-dimensional DNA nanostructure and the form of DNA affect the permeability of the barrier. Using L-form DNA and D-form DNA, the inventors fabricated DNA nanostructures with tetrahedral, triangular prismatic, and hexagonal structures and confirmed the BBB permeability of the nanostructures. As a result, the inventors found that DNA nanostructures with a cube structure exhibited high BBB permeability. Meanwhile, they found that DNA nanostructures fabricated with D-form DNA showed higher brain distribution characteristics than those manufactured with L-form DNA, when administered systemically.

Accordingly, the inventors provide a DNA nanostructure having a cube structure as a drug delivery system for the purpose of delivering a drug into the brain.

The present inventors investigated whether a therapeutic effect on brain tumors could be obtained by loading a drug into D-Cb nanostructures that exhibit high brain distribution characteristics upon systemic administration. Specifically, ASO targeting PLK1 mRNA was loaded into glioblastoma-induced mice by adding it to the 3-terminus of the S6 strand of D-Cb. In this specification, D-Cb loaded with ASO is denoted as ASO@D-Cb. ASO@D-Cb exhibited high brain distribution characteristics upon intravenous administration to mice, which were not affected by ASO loading. Further, ASO loaded into DNA nanostructures was incorporated into glioblastoma cells and the expression level of the target molecule, i.e. PLK1, was down-regulated.

Accordingly, the present disclosure provides a pharmaceutical composition for treating brain tumors by loading a drug capable of inhibiting the proliferation of cancer cells or inducing apoptosis into the above-described DNA nanostructures.

As used herein, “brain tumors” may refer to gliomas, astrocytomas, malignant astrocytomas, glioblastomas, etc.

In the present disclosure, the drug may be an oligonucleotide targeting a gene differentially expressed in normal cells and cancer cells, and may include an RNAi, ASO (antisense oligonucleotide), ADAR (adenosined deaminases acting on RNA), etc., that may down-regulate the expression of the target gene by including a sequence complementary to part or all of the target gene.

Meanwhile, the drug delivery system provided by the present disclosure consists of DNA, enabling loading of hydrophobic anticancer drugs such as doxorubicin. Therefore, the drug may be an anticancer agent with hydrophobic properties among the known anticancer agents.

Hereinafter, specific embodiments will be described in detail with reference to the accompanying drawings, although the present disclosure may have various embodiments and various changes may be made to the embodiments. Such illustrations and descriptions are not intended to limit the present disclosure to specific embodiments, and should be understood as including all transformations, equivalents, and substitutes that may be included in the spirit and scope of the present disclosure. In the description of the present disclosure, any detailed description of well-known related structures or functions has been omitted where it is deemed that such description will cause ambiguous interpretation of the present disclosure.

### Experimental Methods and Materials

### 1. Materials

All quantitative RT-PCR (qRT-PCR) primers and phosphoramidites required for D-DNA synthesis were purchased from Bioneer (Daejeon, Korea). The phosphoramidites for L-DNA synthesis and the CPG for 3′ modifications (3′-FAM and 3′-NH₂) were purchased from Glen Research (Sterling, VA, USA). Streptavidin-coated magnetic beads (Dynabeads™ MyOne Streptavidin™ T1) and the SYBR green Master Mix used for qRT-PCR were obtained from Thermo Fisher Scientific (Waltham, MA, USA). The BALB/c mice used in the study, specifically male individuals aged 5 weeks, were provided by Orient Bio Inc. (Seongnam, Korea). Various additional buffers, organic solvents, and chemical reagents essential for the experimental procedures were purchased from Biosesang (Seongnam, Korea), Samchun Chemicals (Seoul, Korea), and Sigma Aldrich (Missouri, USA), respectively. The RNeasy Mini kit was obtained from Qiagen (Hilden Germany), while the cDNA synthesis kit was purchased from Enzynomics (Daejeon, Korea). The antibodies employed in western blotting and flow cytometry were purchased from Cell Signaling Technology (Danvers, MA, USA), Santa Cruz Biotechnology (Dallas, TX, USA), Abcam (Cambridge, UK), R&D Systems (Minneapolis, MN, USA), Bethyl Laboratories Inc. (Montgomery, TX, USA), and Biolegend (San Diego, CA, USA).

### 2. Statistical Analysis

The data is presented as the mean±standard error of the mean (SEM). To assess the significance of observed differences, statistical analysis was conducted through one-way analysis of variance (ANOVA) using Tukey's multiple comparison test by GraphPad Prism software. For this analysis, data with *p<0.05, **p<0.01, ***p<0.001, and ****p<0.0001 is considered statistically significant.

### 3. OT Synthesis

Oligonucleotides were synthesized at a 1 μmol-scale using the Mermaid-4 DNA/RNA synthesizer (Bioautomation, MN, USA) with conventional phosphoramidite chemistry. After synthesis, the oligonucleotides were cleaved from CPG and deprotected in 33% aqueous ammonia at 55° C. for 17 hours. Subsequent purification was achieved through denaturing polyacrylamide gel electrophoresis (PAGE), followed by ethanol precipitation. The molecular weight of all of the purified strands was characterized via ESI-MS analysis, conducted by Novatia Inc. (Pennsylvania, USA).

### 4. Preparation of Self-Assembled DNA Nanostructures

The solution containing oligonucleotides (S1-S6, 1 μM, Table 1) in TM buffer (5 mM MgCl₂, 10 mM Tris-HCl, pH 8.3) was heated to 95° C. for 10 minutes and slowly cooled down to 4° C. for 24 hours. The self-assembled structures were verified by native PAGE (10%) in 0.5×TBE buffer. The bands were visualized using 3′-FAM incorporated in the S1 strand. The gels were imaged using the iBrightFL1000 system (Thermo Fisher Scientific).

TABLE 1

| | | | SEQ |
|---|---|---|---|
| | | | ID |
| | | Sequence (5′ to 3′) | NO. |
| Td | S1 | GGG ATC CCG ATT CGA | 1 |
| | | GAC AGC ATT TCT CCC | |
| | | ACA C | |
| | S2 | CGT GGT AGG TTT TGC | 2 |
| | | TGT CTC GTT AGC GCC | |
| | | GGC C | |
| | S3 | TCG GGA TCC CTT CAC | 3 |
| | | GGG CAA CTT GGC CGG | |
| | | CGC T | |
| | S4 | ACC TAC CAC GTT GTT | 4 |
| | | GCC CGT GTT GTG TGG | |
| | | GAGA | |
| Tp | S1 | TGC TGT CTC GTT CGT | 5 |
| | | GGT AGG TTT GCA GAA | |
| | | GGT C | |
| | S2 | CGA GAC AGC ATT CAC | 6 |
| | | GGG CAA CTT TCT CCC | |
| | | ACA CTT GGG AAA GGT | |
| | | C | |
| | S3 | GTG TGG GAG ATT CCG | 7 |
| | | AGG GAT CTT GCG GAT | |
| | | TGT A | |
| | S4 | GGC CGG CGC TTT ACC | 8 |
| | | TAC CAC GTT GAC CTT | |
| | | TCC CTT TAC AAT CCG | |
| | | C | |
| | S5 | AGC GCC GGC CTT GAT | 9 |
| | | CCC TCG GTT GTT GCC | |
| | | CGT GTT GAC CTT CTG | |
| | | C | |
| Cb | S1 | GTG TGG GAG ATT AGT | 10 |
| | | CAT TAA GTT TAC AAT | |
| | | CCG CTT GTA ATC GTA | |
| | | G | |
| | S2 | GTT GCC CGT GTT CTA | 11 |
| | | CGA TTA CTT GGT CGG | |
| | | GAA ATT CGT GGT AGG | |
| | | T | |
| | S3 | GCG GAT TGT ATT TAG | 12 |
| | | GGG ACA TTT CGA GAC | |
| | | AGC ATT TTT CCC GAC | |
| | | C | |
| | S4 | GCA GAA GGT CTT CCG | 13 |
| | | AGG GAT CTT ACC TAC | |
| | | CAC GTT TGC TGT CTC | |
| | | G | |
| | S5 | AGC GCC GGC CTT TCT | 14 |
| | | CCC ACA CTT CAC GGG | |
| | | CAA CTT GAT CCC TCG | |
| | | G | |
| | S6 | CTT AAT GAC TTT GGC | 15 |
| | | CGG CGC TTT GAC CTT | |
| | | CTG CTT ATG TCC CCTA | |

### 5. Dynamic Light Scattering (DLS)

The hydrodynamic sizes of DNA nanostructures (250 nM in TM buffer) were determined by dynamic light scattering (DLS) using a Zetasizer instrument (Malvern Instruments, Worcestershire, UK).

### 6. Atomic Force Microscopy (AFM)

DNA nanostructures assembled within the concentration range of 50-100 nM were suitably diluted to 20 nM in 1×TAE-Mg buffer (composed of 50 mM Tris-acetate, 2 mM EDTA, 12.5 mM MgCl₂). This diluted solution was then combined with an equal volume of 1×TAE-Mg containing 10 mM NiCl₂. The mixture was deposited onto mica surfaces pretreated with NiCl₂ and subsequently incubated for 1 minute at room temperature. Then, the samples were imaged using a non-contact mode on an atomic force microscopy (AFM) platform (ScanAsyst Multimode, Bruker, USA), employing ScanAsyst-Fluid+ tips (Bruker, USA) in a fluidic environment.

### 7. Serum Stability of DNA Nanostructures

DNA nanostructures (FAM-labeled, 1 μM) were incubated in 50% mouse serum for 0, 1, 4, 7, and 24 hours at 37° C. At each time point, the enzymatic reaction was quenched by adding loading buffer (95% formamide in 0.5 M EDTA), which was followed by a treatment step of heating at 95° C. for 10 minutes. Subsequently, the samples were subjected to agarose (1%) gel electrophoresis, run in 0.5×TBE buffer at 120 V for 1 hour. The bands were imaged using an iBright FL1000 imaging system.

8. Pull-Down of Serum Proteins with D-Cb and L-Cb

Streptavidin-coated magnetic beads or biotinylated D-Cb and L-Cb (1 μM) immobilized onto the streptavidin-coated magnetic beads (20 μL) were washed three times with 1× phosphate-buffered saline (PBS) (100 μL) and then incubated in mouse serum (50 μL, Sigma-Aldrich) in a binding buffer (50 μL, 2×PBS) at 37° C. for 1 hour. The supernatant was removed. The beads were washed three times with 1×PBS. The beads were resuspended in loading buffer (50 mM Tris-HCl, pH 6.8, 2% SDS, 6% v/v glycerol, 2 mM DTT and 0.01% w/v bromophenol blue) and incubated at 95° C. for 10 minutes. The mixture was subsequently analyzed by 5-12% SDS-PAGE. The gel was stained with Coomassie Blue, and then imaged using an iBrightFL1000 system.

### 9. Proteomic Analysis

Proteins that were pulled down through three independent beads, D-Cb, and L-Cb binding experiments, respectively, were separated based on the molecular weight using 5-12% SDS-PAGE. After the gel was stained with Coomassie Blue, the stained gel for each lane of the samples that were pulled down with three types of beads was divided into eight slices and the proteins contained in each gel slice were subjected to tryptic digestion. The proteins were first reduced with 10 mM DTT in 25 mM NH₄HCO₃ for 1 hour at 56° C. They were then alkylated with 55 mM iodoacetamide in 25 mM NH₄HCO₃ for 1 hour at 25° C. in the dark, and subjected to trypsin digestion overnight. Peptides were then extracted with 67% acetonitrile (ACN)/5% formic acid (FA) in water and dried in a miVAC vacuum concentrator (Genevac Ltd., Ipswich, UK). Then, they were resuspended with 20 μL of 0.4% acetic acid. For mass spectral analysis, 13.5 μL of each sample was injected into a reversed-phase Magic C18AQ column (15 cm×75 μm) on an Eksigent MDLC system (Eksigent Technologies, CA, USA). Three replicates of each type of sample were analyzed using mass spectrometry (MS).

The operating flow rate was 350 nL/min, under the following gradient conditions: 0 min 100% buffer A (100% water with 0.1% FA) and 0% buffer B (100% ACN with 0.1% FA), 0-5 min 0-8% B, 5-85 min 8-30% B, 85-90 min 30-70% B, 90-100 min 70% B, 100-110 min 70-2% B, and 100-120 min 2% B. The nano HPLC system was coupled to an LTQ XL-Orbitrap mass spectrometer (Thermo Fisher Scientific, MA, USA). The spray voltage was set to 2.5 kV and the temperature of the heated capillary was set to 250° C. Survey full-scan mass spectrometry (MS) spectra (300-2000 m/z) were acquired with 1 micro scan at a resolution of 60,000, allowing a preview mode for precursor selection and charge-state determination. Tandem mass (MS/MS) spectra for the ten most intense ions were acquired in an ion trap with the following options: isolation width, 2 m/z; normalized collision energy, 35%; dynamic exclusion duration, 30 s. Precursors with +1 charge and unassigned charge states were discarded during data-dependent acquisition. A search of each LC-MS/MS file was run against the SwissProt mouse database (June 2022) having 17132 entries using Proteome Discoverer software (version 2.4, Thermo Fisher Scientific, Bremen, Germany). The search criteria were set to a mass tolerance of 15 ppm for MS data and 0.5 Da for MS/MS data with fixed modification of carbamidomethylation of cysteine (+57.021 Da) and variable modification of methionine oxidation (+15.995 Da). The false discovery rate (FDR) was set at 0.01 for the identification of peptides and proteins. All proteins were identified by two or more unique peptides. For label-free quantitative analysis performed using the three replicates of each type of sample, normalized abundance values of proteins in each sample were obtained from peak area normalized by total peptides using Minora algorithm-based label-free quantification by Proteome Discoverer 2.4. Statistical analysis of the normalized abundance values obtained from the label-free quantification was performed using Perseus software (1.6.14.0). Normalized abundance values were log-transformed, and then missing values were replaced using values computed from the normal distribution with a width of 0.3 and a downshift of 1.8. Proteins exhibiting statistical significance among samples pulled down with beads, D-Cb, and L-Cb were obtained by ANOVA comparison of the log₂ (normalized abundance) values obtained from the three replicates of each type of sample. P-value <0.05 was considered statistically significant. For hierarchical clustering of proteins showing statistically significant changes (P-value <0.05) among samples pulled down with beads, D-Cb, and L-Cb, the abundance values were first normalized using z-score and then clustering of both columns and rows was pursued based on Euclidean distance using the average linkage method using Perseus (1.6.14.0).

### 10. Cellular Uptake Experiments

U87MG cells (Korean Cell Line Bank, Seoul, Korea) were seeded at a density of 5×10⁴ cells per well in a 24-well plate. After 24 hours, the cells were washed twice with PBS and were subsequently treated with Cy5.5-labeled ASO or ASO@D-Cb (50 nM) in 10% fetal bovine serum (FBS)-supplemented Dulbecco's Modified Eagle Medium (DMEM; Welgene, Gyeongsan, Korea) at 37° C., in a 5% CO₂ environment. After 6 hours, the cells were washed three times, suspended in ice-cold PBS (500 μL), and analyzed by flow cytometry (Guava, Millipore, Massachusetts, USA). A minimum of 10,000 cells were examined in triplicate for each sample. For confocal microscopic imaging, cells (2.5×10⁴) were cultured on glass-bottomed dishes and treated with Cy5.5-labeled ASO or ASO@D-Cb (50 nM) for 6 hours. Subsequently, the cells were stained with Hoechst 34580 (1 μg/mL, Thermo Fisher Scientific) for 5 minutes and washed with PBS twice. The cellular fluorescence signals were visualized using confocal microscopy (LSM 800, Carl Zeiss, Jena, Germany).

To elucidate receptor-mediated cellular uptake mechanisms, bEnd.3 cells or U87MG cells were pretreated with 10 μg/mL of antibodies (anti-TfR antibody, anti-LRP1 antibody, anti-LRP2 antibody, or anti-LDLR antibody) for 1 hour and afterwards were treated with FAM-labeled D-Cb or L-Cb for 6 hours. Then, the uptake efficiency was analyzed by flow cytometry.

### 11. In Vitro BBB Permeability Assay

To evaluate the in vitro BBB penetration of DNA nanostructures, the inventors carried out a penetration study using an in vitro BBB monolayer model. Brain endothelial bEnd.3 cells were seeded at a density of 3.0×10⁴ cells on Transwell permeable inserts (0.4 μm, 6.4 mm in diameter; Falcon®, Corning, NY, USA) with 10% FBS-containing DMEM and incubated at 37° C. in a 5% CO₂ atmosphere for one week. The integrity of the BBB model was confirmed by transendothelial electrical resistance (TEER) measurements using EVOM2 (World Precision Instruments, Sarasota, FL, USA). When the TEER value was above 250Ω, the Transwell insert was washed with PBS, and 200 μL of complete medium containing FAM-labeled DNA nanostructures (1 μM) was added to the apical side of the culture insert. The basolateral compartment of the insert was filled with 500 μL complete medium. The complete medium with DNA nanostructures was collected from the basolateral side, and the basolateral part was refilled with fresh complete medium at pre-determined times (0.5, 1, 2, and 4 hours). The fluorescence intensity of collected samples was measured with a fluorescence spectrometer (Multi-detector microplate reader, Synergy H1 Hybrid, BioTek). The DNA nanostructures in the samples were quantified. Then, the apparent permeability (Papp), indicating the in vitro BBB permeability, was calculated using the following equation:

$${p}_{app} = ( {C}_{receiver} \times {V}_{receiver} ) / ( A \times t \times {C}_{donor} ) . \qquad [ Equation 1 ]$$

where C_{donor}, C_{receiver}, V_{receiver}, A, and t denote the concentration of DNA nanostructures in the donor (apical side), the concentration of DNA nanostructures in the receiver (basolater side), receiver volume, membrane area, and incubation time, respectively.

To investigate the receptor-mediated BBB transcytosis efficiency of D-Cb, a transpenetration study with TfR, LRP1, LRP2, LDLR antibodies was performed using an in vitro BBB monolayer model. On the day of the assay, the cells in the insert were pretreated with each antibody in serum-containing medium (10 μg/mL) for 1 hour at 37° C. After 1 hour of incubation, complete medium containing FAM-labeled D-Cb (1 μM) was added to the apical side. Samples were collected from the basolateral side at predetermined times and BBB permeability calculations were performed as described above.

### 12. In Vitro Blood-Tumor Barrier (BTB) Penetration Assay

In vitro U87MG-cellular uptake analysis after BBB penetration (BTB penetration assay) was performed using an in vitro BBB co-culture model. Briefly, bEnd.3 cells were seeded at a density of 3.0×10⁴ cells into the Transwell permeable inserts (apical chambers) and incubated for 5 days. Then, U87MG cells were seeded at a density of 3.0×10⁴ on the coverslip in the basolateral chambers. After 2 days, 200 μL of complete medium containing FAM-labeled DNA nanostructures (1 μM) was added to the apical side of the culture insert. The basolateral compartment of the insert was filled with 500 μL complete medium. The U87MG cells in the basolateral chambers were incubated at 37° C. for 4 hours, washed four times with PBS, stained with DAPI for 5 minutes, and then fixed using a 2% PFA solution. The coverslips were mounted with a mounting solution on the slide glass and then fluorescence intensity was observed under a confocal microscope (A1Plus, Nikon). The relative fluorescence intensity was determined by normalizing the intensity ratio FAM (Cb)/DAPI (nuclei).

### 13. Preparation of Orthotopic GBM Mice

All animal experiments were approved by the Institutional Animal Care and Use Committee at Gachon University (Approval number: LCDI-2022-0076). The U87MG-fLuc-GFP cell line was kindly provided by Dr. Kwang Il Kim from the Korea Institute of Radiological and Medical Sciences. Orthotopic GBM-xenografted mice were prepared using U87MG-fLuc-GFP cells following a previously established protocol. Briefly, male athymic BALB/c nude mice (5 weeks old, 20-30 g) were placed on a stereotactic device (Harvard Apparatus, MA, USA) using ear bars under 1-1.5% isoflurane inhalation anesthesia. U87MG-FLuc-GFP cells (1×10⁵ cells) suspended in 2 μL of PBS were injected into the right striatum at a flow rate of 0.5 μL/min through a tiny hole on the skull at coordinates 2.0 mm laterally, 0.2 mm anteriorly, and 3.2 mm ventrally from the bregma. Brain tumor growth was monitored by bioluminescence intensity (BLI).

### 14. In Vivo Imaging

All experimental procedures involving living animals were strictly conducted in accordance with the relevant regulations and institutional protocols of the Korea Institute of Science and Technology (Approval Number: IACUC-2022-047-2). Cy5.5-labeled DNA nanostructures or ASO (2 μM, 200 μL) were intravenously administered to healthy BALB/c or GBM mice. In vivo fluorescence images were acquired using an IVIS Imaging Spectrum System, utilizing an emission wavelength of 700 nm and an excitation wavelength of 640 nm. The data was subsequently analyzed with IVIS Living Imaging 3.0 software. For ex vivo imaging, at 2 hours post injection, the mice were euthanized, and the organs were extracted and imaged via the IVIS system.

### 15. Analysis for Biodistribution of D-Cb and L-Cb

At 2 hours post injection of D-Cb or L-Cb, the mice (BALB/c or GBM) were transcranially perfused with 10 mM potassium PBS at a flow rate of 1 mL/min (1 mL) to remove the nanostructures in the blood, and the brain was harvested. To estimate brain distribution levels (injected dose per gram of tissue: ID %/g) of D-Cb and L-Cb labeled with FAM or Cy5.5, the excised brain tissues were homogenized under cryogenic conditions and were subsequently lysed in RIPA buffer. The lysates were centrifuged (12,000 rpm, 10 minutes, 4° C.), and fluorescence intensity of the supernatant from each sample was measured using a plate reader (Victor Nivo, Perkin Elmer, USA) with a specific filter set (Ex 640 nm, Em 685 nm) to quantify the total amount of fluorescence-labeled structures. To quantify intact oligonucleotides, the brain lysates were analyzed using 10% denaturing PAGE (7 M urea). Reference quantities of oligonucleotides (Cy5.5-labeled; 0.032, 0.64, 0.125, 0.25, and 0.5 pmol) were collaterally loaded onto the gel. The quantity of intact oligonucleotides present was estimated based on the intensity of the gel bands, which were subsequently quantified by ImageJ software. Using the determined amount of intact oligonucleotides within each lysate, the amount of intact D-Cb and L-Cb in the brain was calculated, accounting for the total lysate volume and the tissue weight.

### 16. Brain Section Imaging

The brains of GBM mice were excised 2 hours after intravenous injection of Cy5.5-labeled D- or L-Cb. The harvested brains were embedded with optimal cutting temperature (OCT) compound (Leica Biosystems, Germany) and frozen in a rapid freezer. The frozen tissue blocks were sectioned into 15 μm sections by Lab Core Incorporation (Seoul, Korea). The section slides were washed with PBS, mounted with DAPI containing-mounting solution (Abcam, UK) and imaged using confocal microscopy (LSM 800).

### 17. ESI-MS Detection of Intact Oligonucleotides in Brain Lysates

Biotinylated D-Cb (2 μM, 200 μL) was administered intravenously to BALB/c mice. After 2 hours, brain tissues were collected and subsequently homogenized under cryogenic conditions, and lysed in RIPA buffer. The lysed tissue samples were then centrifuged at 12,000 g for 10 minutes at 4° C., and the supernatant was collected as the brain tissue lysate. Streptavidin-coated magnetic beads (30 μL) were washed with the buffer (0.5 mM EDTA, 1 M NaCl, 5 mM Tris-HCl, pH 7.5) three times and incubated with the tissue lysate at 37° C. for 2 hours. The beads were washed with PBS and 50 mM NaOH. Biotinylated S5 strands were eluted by incubating the beads with D-biotin (1 mM) in distilled water at 95° C. for 20 minutes. D-biotin was removed by filtering the eluted solution using a G25 filtration column. The filtrate was analyzed using electrospray ionization mass spectrometry (ESI-MS), which was conducted by Novatia Inc. (Pennsylvania, USA).

### 18. Systemic Brain Delivery of ASO@D-Cb in Orthotopic GBM Mice.

Orthotopic GBM-xenografted mice were intravenously administered with Cy5.5-labeled ASO@D-Cb (200 μL, 400 pmol/mouse, 20 nmol/kg) through the tail vein. After 2 hours, the GBM-bearing mice were transcranially perfused with 10 mM potassium PBS at a flow rate of 1 mL/min (50 mL) and then fixed by a 4% (v/v) paraformaldehyde (PFA) solution at a flow rate of 2 mL/min (50 mL). The brains were collected, divided into 4 mm coronal slices, and then post-fixed with a 4% (v/v) PFA solution overnight at 4° C. The brain coronal slices were sectioned into 40 μm thick sections using a vibratome (Leica V1000S, Germany) and the sections were stained by DAPI (Life Technologies). The stained tumor tissue slides were observed by laser scanning confocal microscopy (LSCM, A1Plus, Nikon, Tokyo, Japan). The fluorescence intensity in the images was quantitatively analyzed using Nikon NIS-E image analysis software.

### 19. Biodistribution of ASO@D-Cb in Orthotopic GBM Mice.

Orthotopic GB mice were intravenously administered with Cy5.5-labeled ASO@D-Cb (200 μL, 400 pmol/mouse, 20 nmol/kg) through the tail vein. Systemic fluorescence images were obtained at predefined time points within 24 hours following intravenous administration using an IVIS optical imaging system (Ami HT imaging system, Spectral Instruments Imaging, Tucson, AZ, USA) equipped with a long wavelength emission filter (675-730 nm).

### 20. In Vivo Efficacy of ASO@D-Cb for Treatment of GBM

The orthotopic GBM mice were weighed and randomly divided into four groups (n=4), and each group was intravenously injected with PBS (200 μL), ASO, ASO-SC@D-Cb, or ASO@D-Cb (400 pmol ASO/mouse) via the tail vein every two days for 10 days. The bioluminescence signal from the brain tumor, generated by intraperitoneal injection of 150 mg/kg D-luciferin, was imaged on every injection day before treatment. An IVIS (Ami HT imaging system) was used to repeatedly acquire the images over 10 minutes at intervals of 2 minutes. The bioluminescence intensity was analyzed using Aura imaging software (Spectral Instruments Imaging). Mice body weights were monitored every 2 days during ASO@D-Cb treatment. After the mice were sacrificed on day 24, the major organs were collected, washed, and fixed with a 4% (v/v) PFA solution overnight at 4° C. The fixed tissues were embedded in paraffin, sectioned, and mounted on glass slides. The sections were stained with hematoxylin and eosin (H&E) and subjected to a transferase-mediated nick end labeling (TUNEL) assay.

### 21. Western Blot

Cell lysates (10 μg) or brain lysates (50 μg) were separated by 12% sodium dodecyl sulfate polyacrylamide gel electrophoresis (SDS-PAGE). After the electrophoresis, the proteins in the SDS-PAGE gel were transferred onto a polyvinylidene fluoride (PVDF) membrane. The membrane was incubated with TBST (10 mM Tris-HCl, pH 8.0, 150 mM NaCl, 0.05% Tween 20) containing 5% skim milk at room temperature for 1 hour and then washed with TBST three times. The membrane was incubated with primary antibodies, specifically anti-PLK1 antibody (dilution 1:1000, Santa Cruz Biotechnology) and anti-GAPDH antibody (dilution 1:1000, Cell Signaling Technology) in TBST overnight at 4° C. The unbound antibodies were washed with TBST three times. Then, the membrane was incubated with a horseradish peroxidase (HRP)-conjugated secondary antibody (dilution 1:10,000, Santa Cruz Biotechnology) in TBST containing 5% skim milk at room temperature for 1 hour. After a rigorous washing the membrane with TBST three times, protein bands were visualized utilizing the Super Signal™ West Pico Chemiluminescent substrate (Thermo Fisher Scientific). The resulting images were captured using an iBright FL1000 imaging system.

22. qRT-PCR

U87MG cells were seeded on a 12-well plate (2×10⁵ cells/well). Cells were treated with ASO, ASO@D-Cb, ASO-SC@D-Cb, PBS, D-Cb, and ASO/Lipofectamine RNAiMax in 10% FBS-supplemented DMEM. After 24 hours, the cellular RNA was extracted using an RNeasy Mini kit (Qiagen). The RNA concentration was quantified by a Nanodrop system (Thermo Fisher Scientific). The cDNA of the cellular RNA (2 μg) was prepared by random hexamer-primed reverse transcription. The cDNA was mixed with 2×SYBR Green Master Mix (Thermo Fisher Scientific) and PLK1 or GAPDH primers (primer sequences are shown in Table 2 below). PCR of the cDNA was performed using a StepOnePlus real-time PCR system (Applied Biosystems). The relative amount of PLK1 transcripts was normalized based on the amount of GAPDH transcripts, calculated with the 2-ΔΔCt method. To estimate PLK1 mRNA levels in the brain lysates, total RNA was extracted from homogenized brain tissues and analyzed using the procedure described above.

TABLE 2

| | | | SEQ |
|---|---|---|---|
| | | | ID |
| Gene | Primer | Sequence | NO. |
| PLK1 | F | GCACAGTGTCAATGCCTCCAAG | 16 |
| | R | GCCGTACTTGTCCGAATAGTCC | 17 |
| GAPDH | F | GTCTCCTCTGACTTCAACAGCG | 18 |
| | R | ACCACCCTGTTGCTGTAGCCAA | 19 |

### Experimental Results

### 1. Preparation and Characterization of Self-Assembled DNA Nanostructures

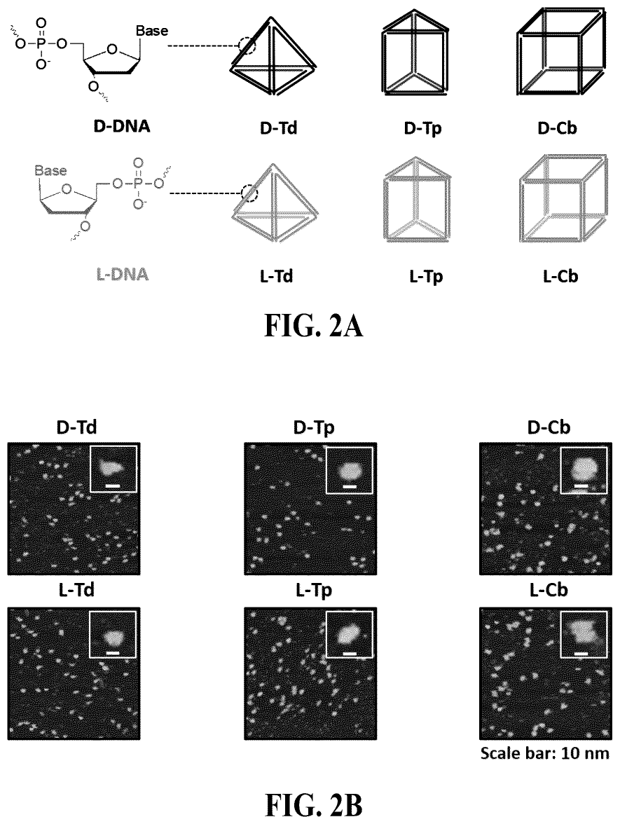

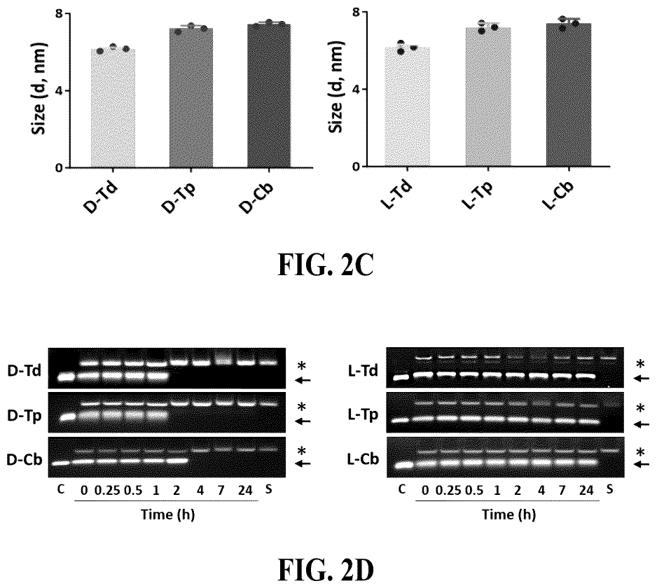

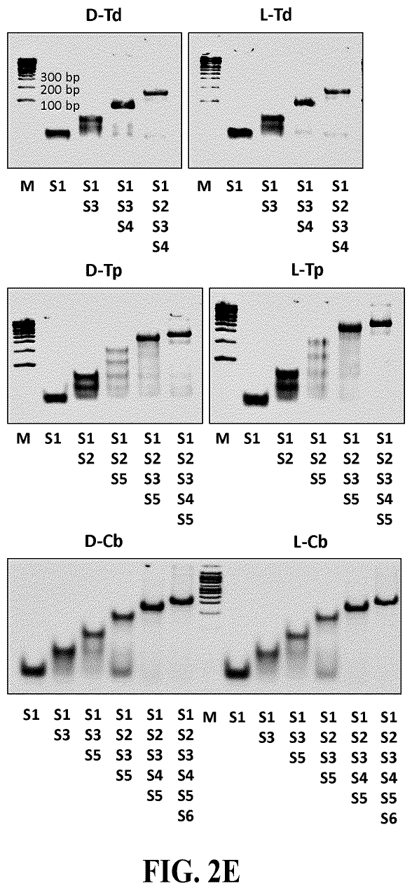

Assuming that small-sized nanocarriers are likely to be absorbed by cerebral microvascular endothelial cells and pass through the BBB, wireframe DNA nanostructures with a 10 bp duplex per side were designed to have one of three shapes: tetrahedron (Td), triangular prism (Tp), and cube (Cb), and two types of deoxyribose backbone: D-DNA and L-DNA (total of six types of DNA nanostructures) (FIG. 2A). The base sequences constituting the wireframes of each DNA nanostructure are shown in Table 1 above, and the DNA nanostructures were prepared via self-assembly of oligonucleotides through a heating and annealing process (Biomaterials 2019; 195: 1-12).

The fabricated six types of DNA nanostructures were characterized by electrospray ionization mass spectrometry (ESI-MS) and the results are shown in Table 3 below.

TABLE 3

| | | | Estimated | | | | | Estimated | |
|---|---|---|---|---|---|---|---|---|---|
| D-DNA | | | [M − H]⁻ | Observed | L-DNA | | | [M − H]⁻ | Observed |
| Td | S1 | 3′FAM | 10946.3 | 10949.3 | Td | S1 | 3′FAM | 10946.3 | 10945.9 |
| | S2 | 3′NH [symbol] | 10642.8 | 10643.5 | | S2 | 3′NH [symbol] | 10642.8 | 10643.4 |
| | S3 | 3′NH [symbol] | 10590.7 | 10590.0 | | S3 | 3′NH [symbol] | 10590.7 | 10590.7 |
| | S4 | 3′NH [symbol] | 10659.8 | 10660.0 | | S4 | 3′NH [symbol] | 10659.8 | 10660.0 |
| Tp | S1 | 3′FAM | 11125.4 | 11128.4 | Tp | S1 | 3′FAM | 11125.4 | 11128.4 |
| | S2 | 3′NH [symbol] | 14283.2 | 14283.6 | | S2 | 3′NH [symbol] | 14283.2 | 14283.8 |
| | S3 | 3′NH [symbol] | 10803.9 | 10804.5 | | S3 | 3′NH [symbol] | 10803.9 | 10804.5 |
| | S4 | 3′NH [symbol] | 14103.0 | 14103.8 | | S4 | 3′NH [symbol] | 14103.0 | 14103.4 |
| | S5 | 3′NH [symbol] | 14222.1 | 14222.3 | | S5 | 3′NH [symbol] | 14222.1 | 14222.3 |
| Cb | S1 | 3′FAM | 14847.9 | 14847.3 | Cb | S1 | 3′FAM | 14847.9 | 14830.6 |
| | S2 | 3′NH [symbol] | 14428.2 | 14428.3 | | S2 | 3′NH [symbol] | 14428.2 | 14428.4 |
| | S3 | 3′NH [symbol] | 14350.2 | 14350.4 | | S3 | 3′NH [symbol] | 14350.2 | 14350.0 |
| | S4 | 3′NH [symbol] | 14287.1 | 14287 | | S4 | 3′NH [symbol] | 14287.1 | 14287 |
| | S5 | 3′NH [symbol] | 14138.0 | 14137.0 | | S5 | 3′NH [symbol] | 14138.0 | 14137.8 |
| | S6 | 3′NH [symbol] | 14179.1 | 14179.4 | | S6 | 3′NH [symbol] | 14179.1 | 14179.3 |

[symbol]

indicates data missing or illegible when filed

The self-assembly of the oligonucleotides was evaluated by polyacrylamide gel electrophoresis (PAGE). DNA nanostructures were imaged by atomic force microscopy (AFM), which roughly revealed the shapes of the DNA nanostructures as designed (FIG. 2B). The hydrodynamic sizes of the DNA nanostructures measured by dynamic light scattering were 6.17±0.12 nm for Tds, 7.22±0.15 nm for Tps, and 7.45±0.11 nm for Cbs (FIG. 2C). The sizes of DNA nanostructures based on the D-DNA backbone (D-Td, D-Tp, and D-Cb) were nearly identical to those based on the L-DNA backbone (6.18±0.20 nm for L-Td, 7.22±0.20 nm for L-Tp, and 7.40±0.21 nm for L-Cb). Serum stability of the DNA nanostructures was also tested. As a result, it was determined that the D-DNA nanostructures degraded in 50% mouse serum after 2-4 hours, while all the L-DNA nanostructures were highly stable in the serum as expected (FIG. 2D).

### 2. Biodistribution of DNA Nanostructures

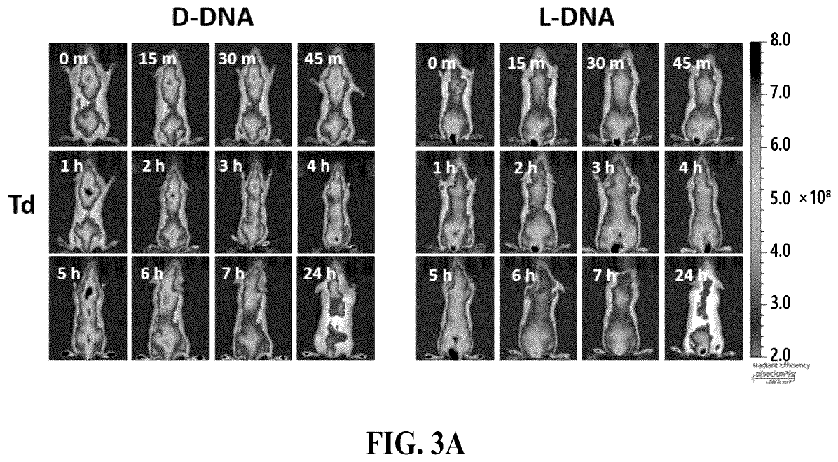

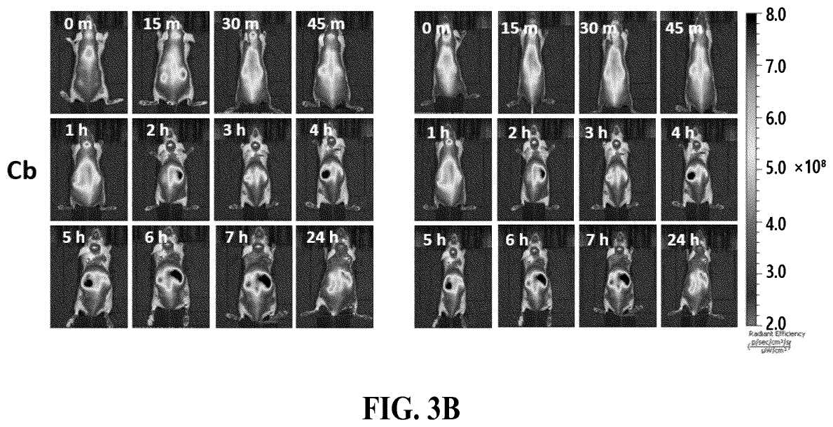

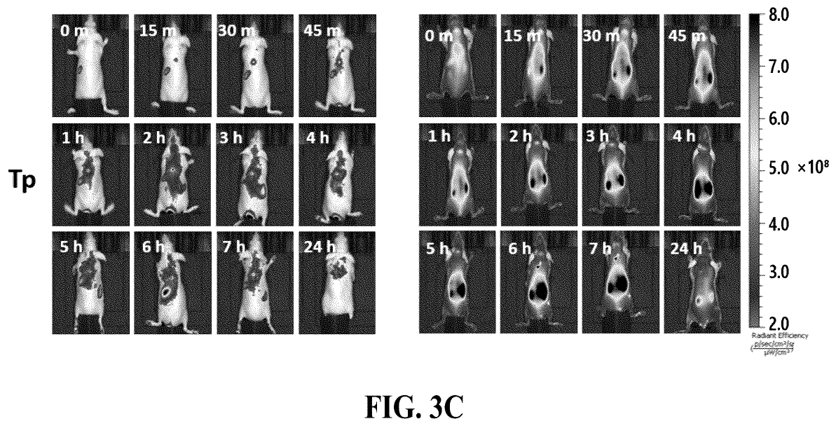

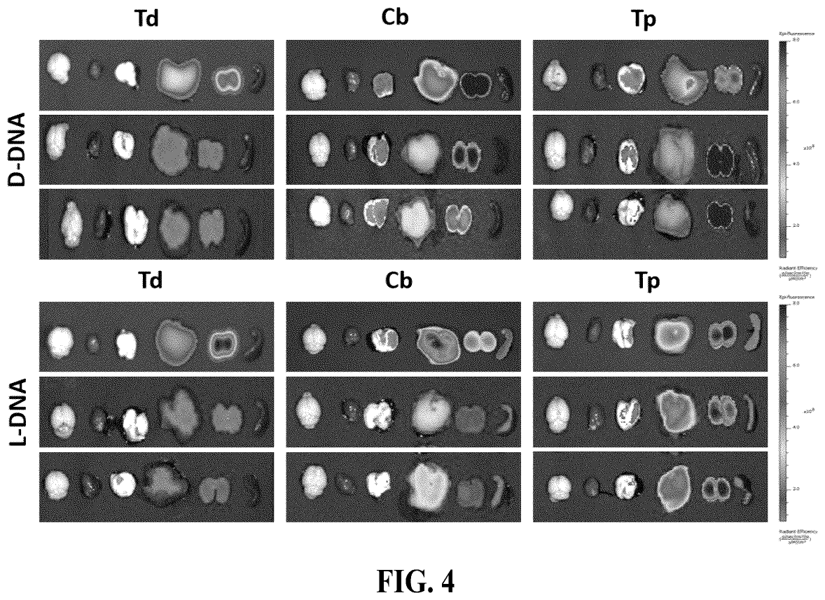

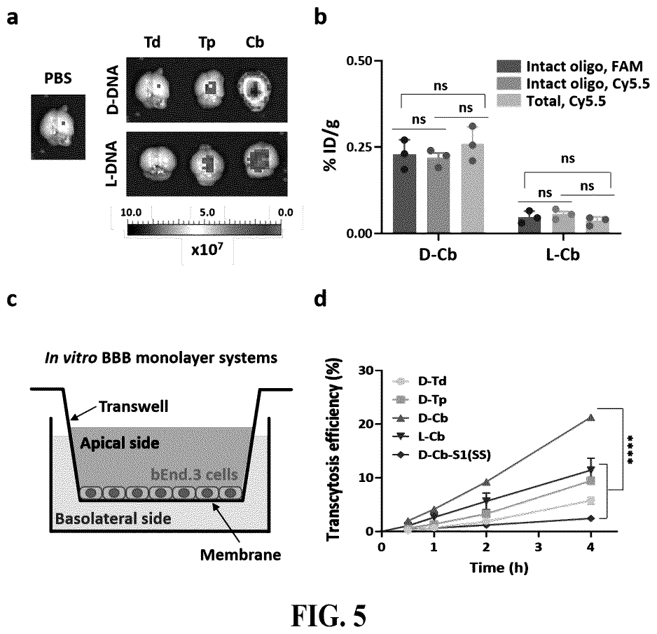

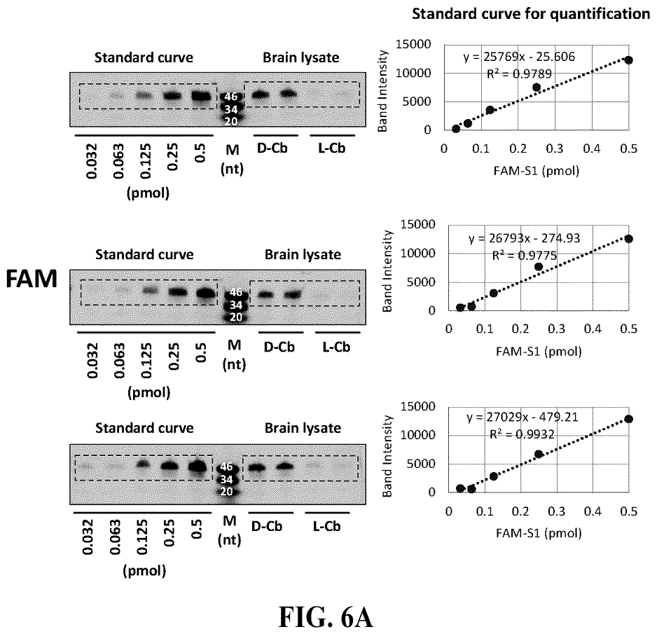

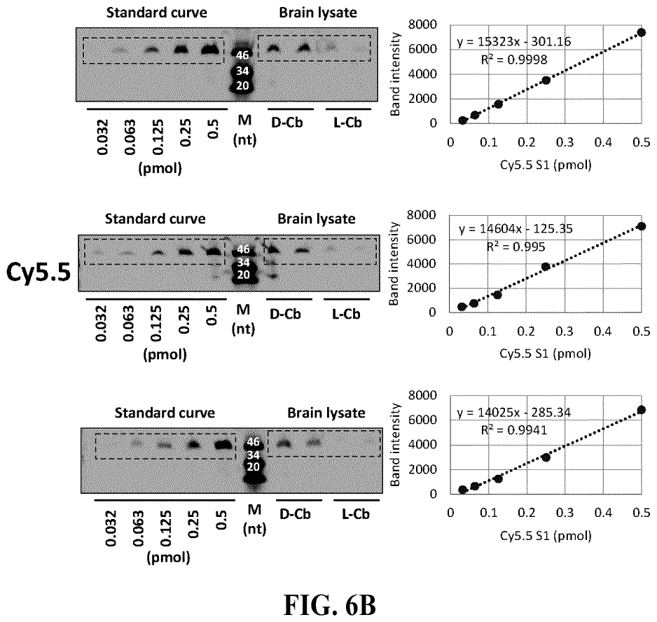

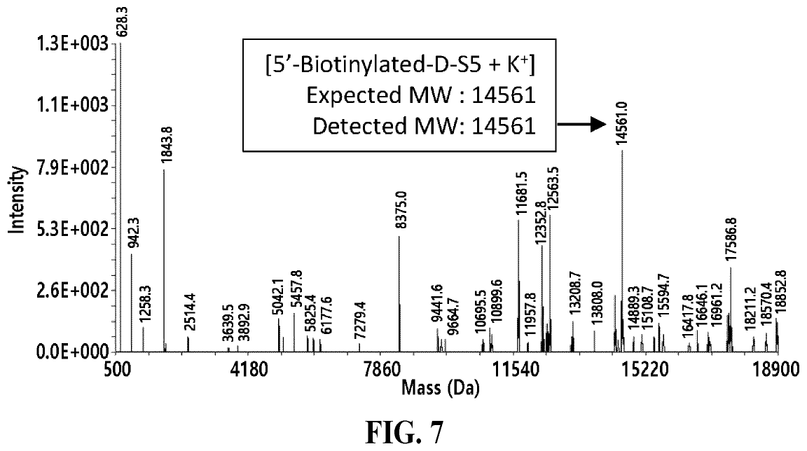

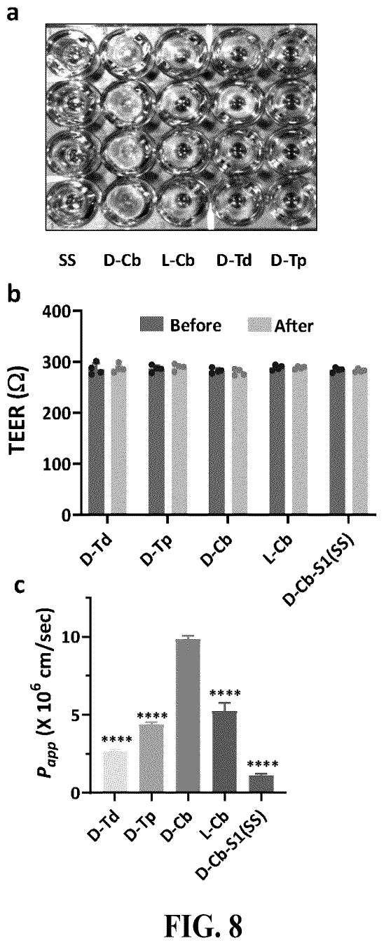

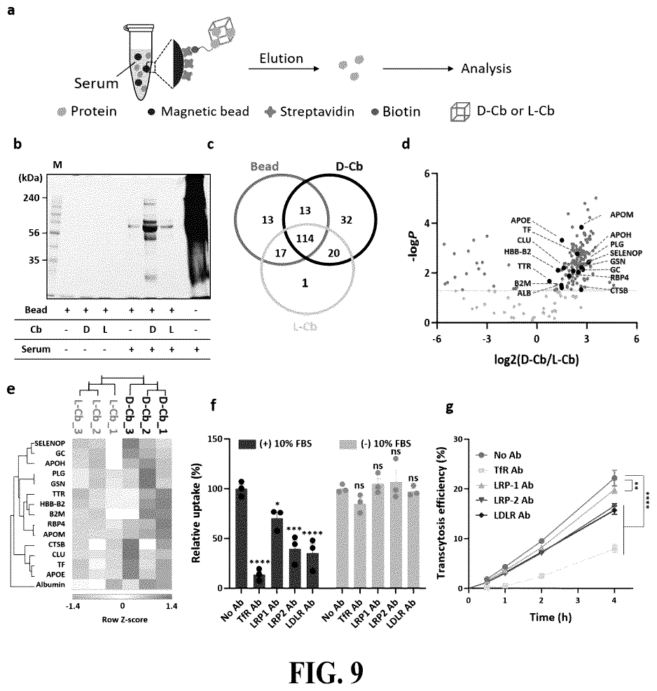

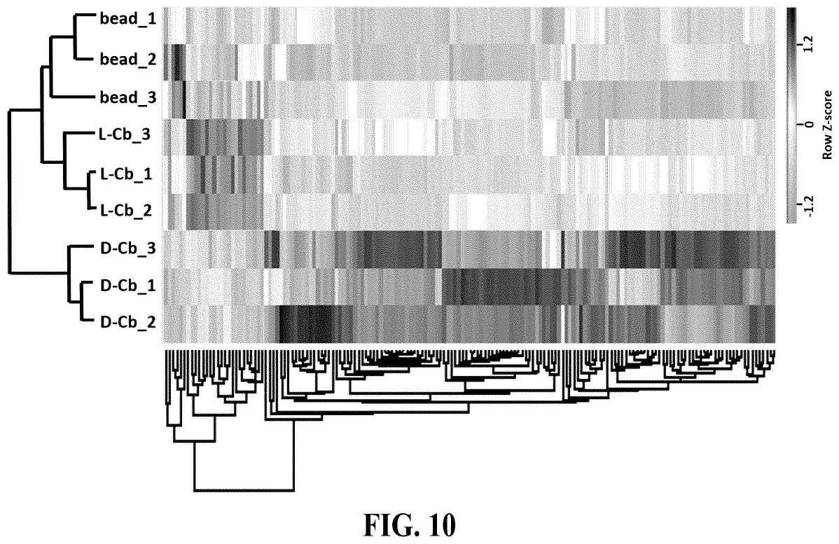

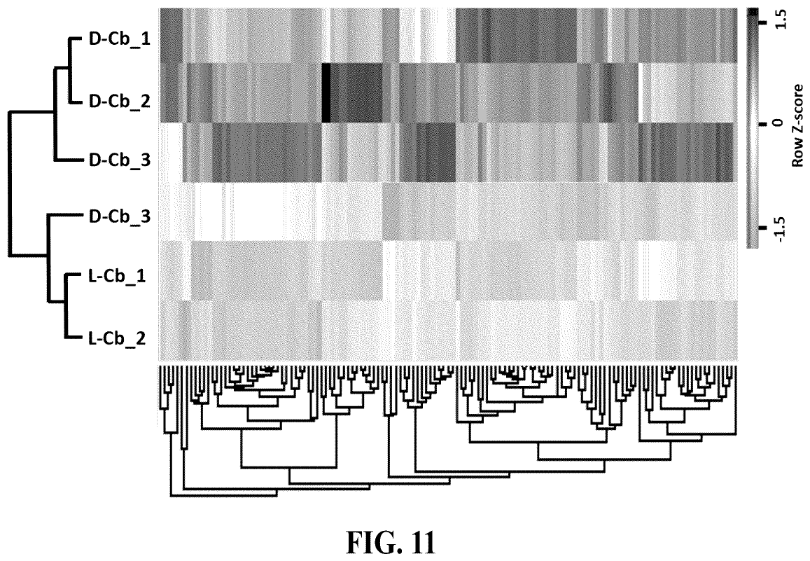

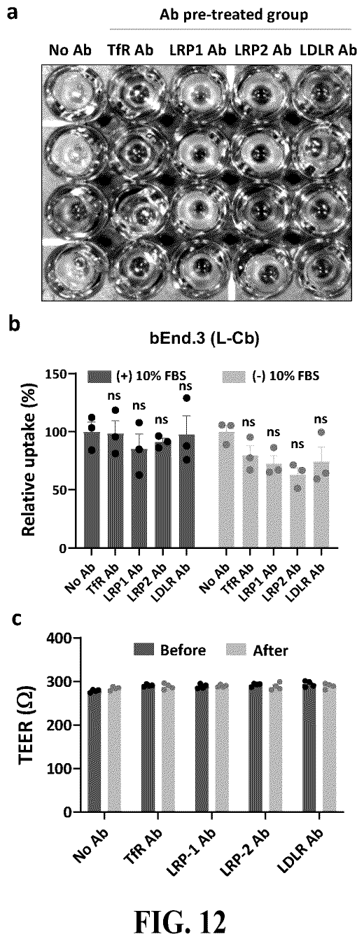

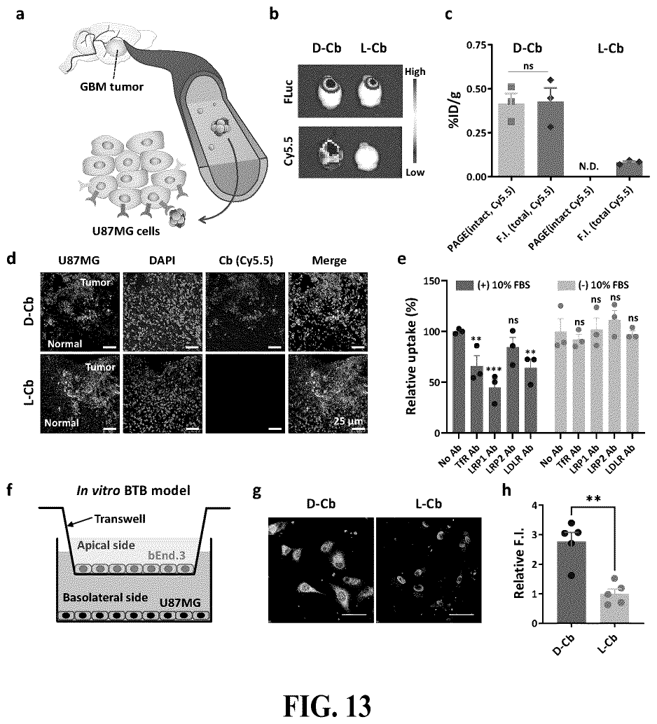

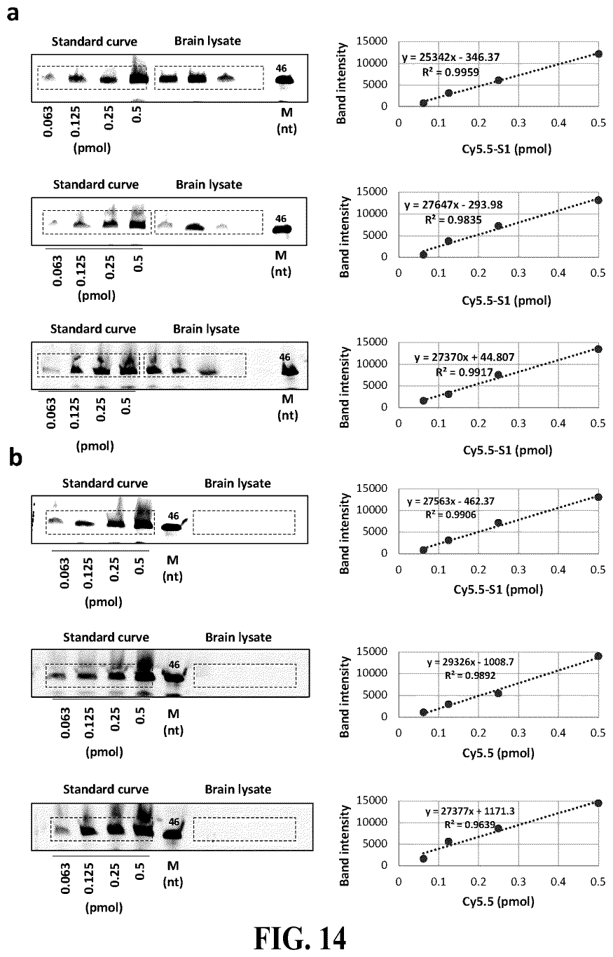

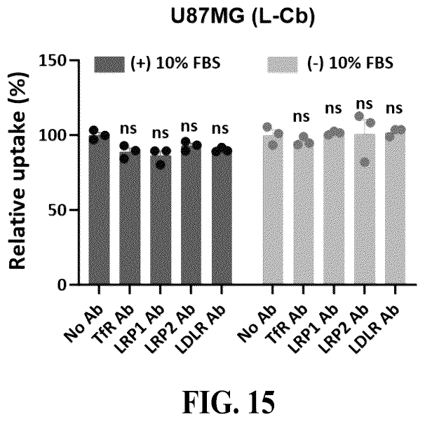

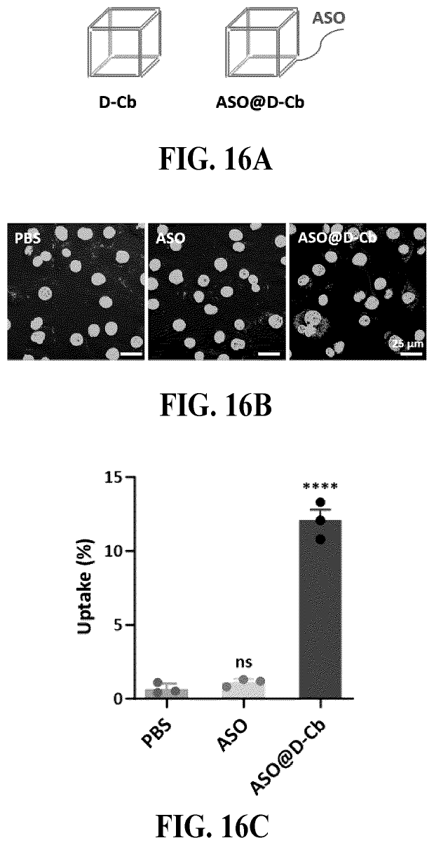

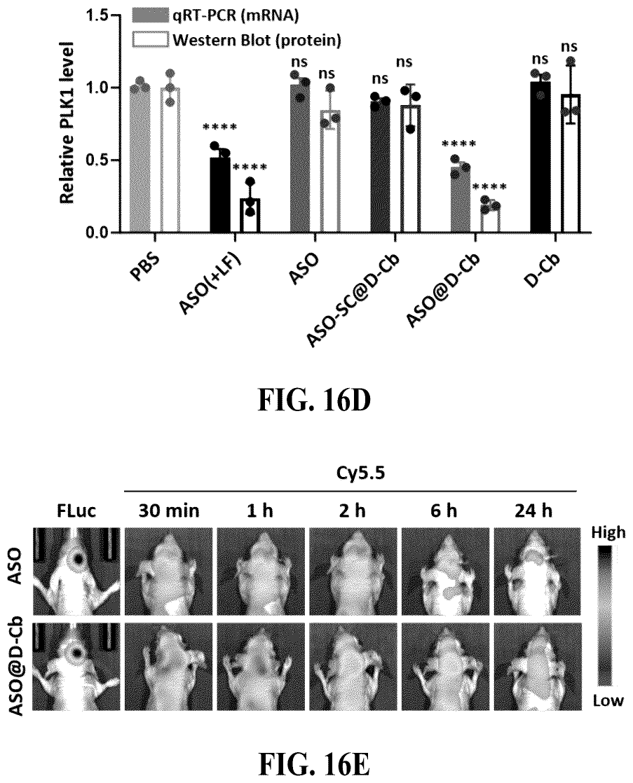

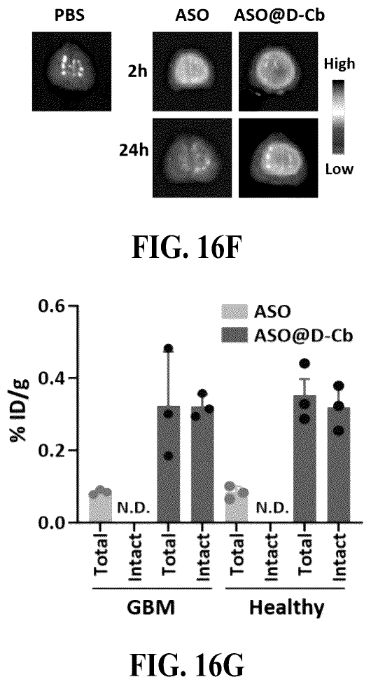

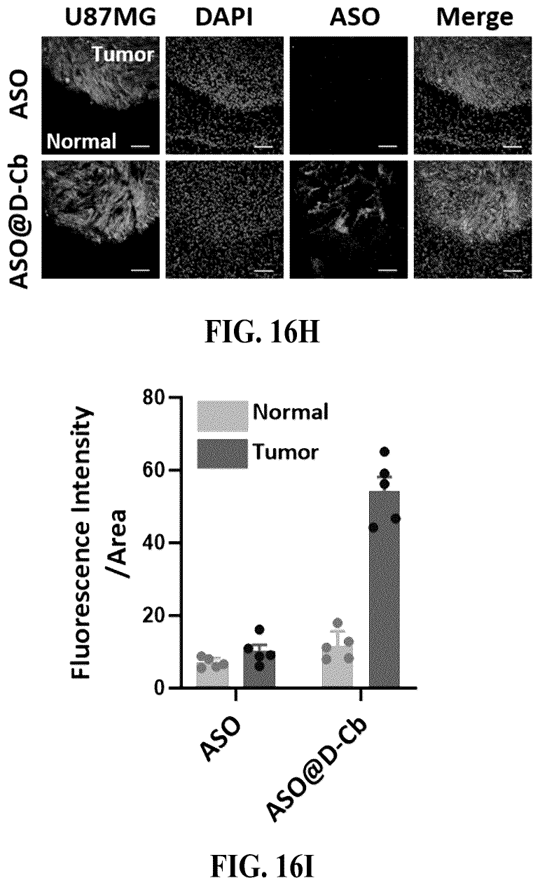

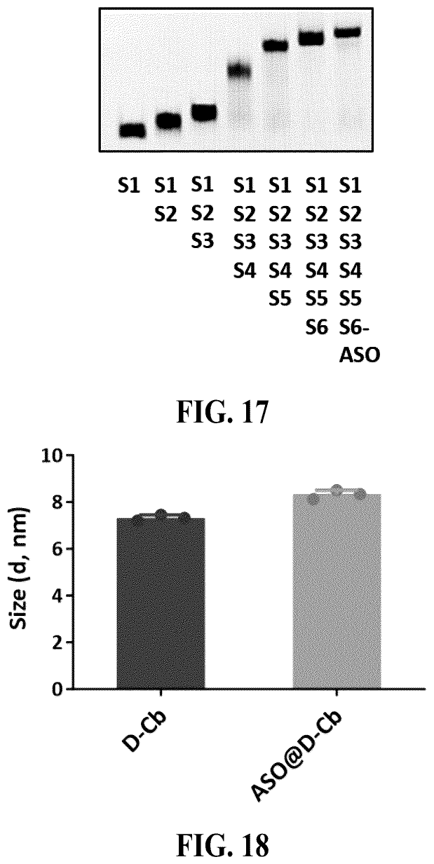

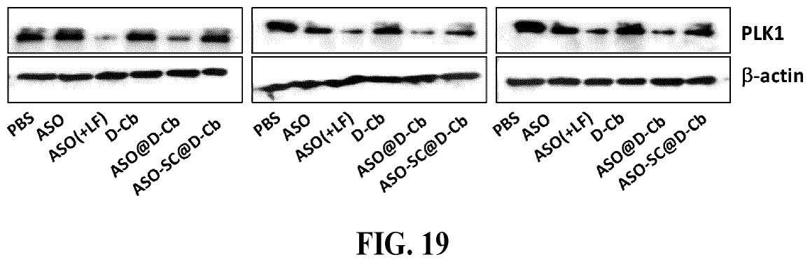

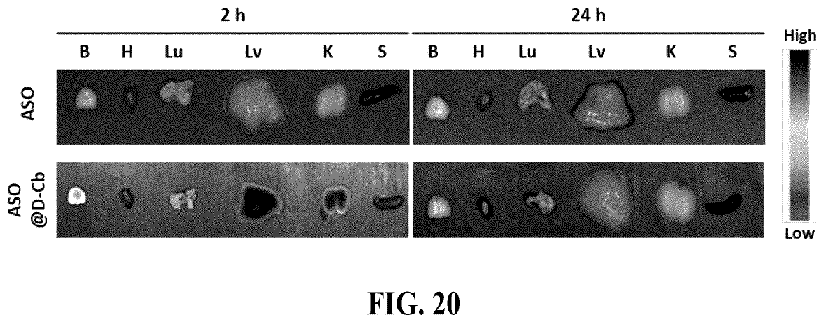

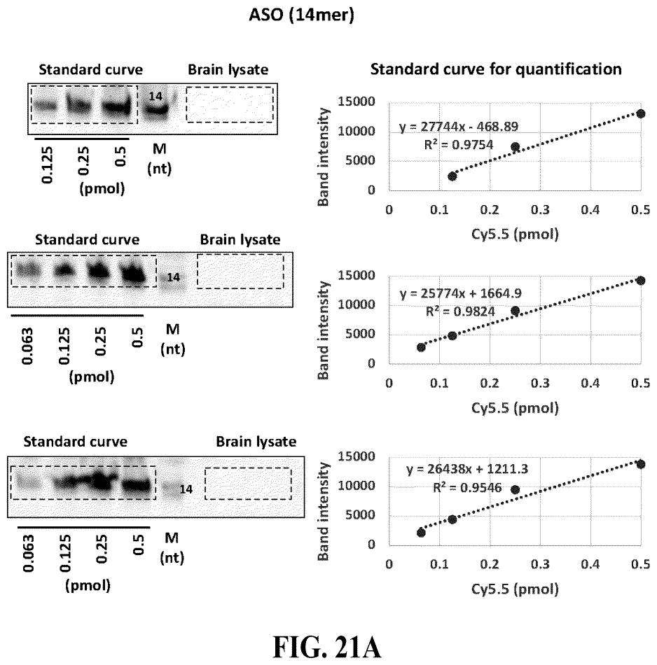

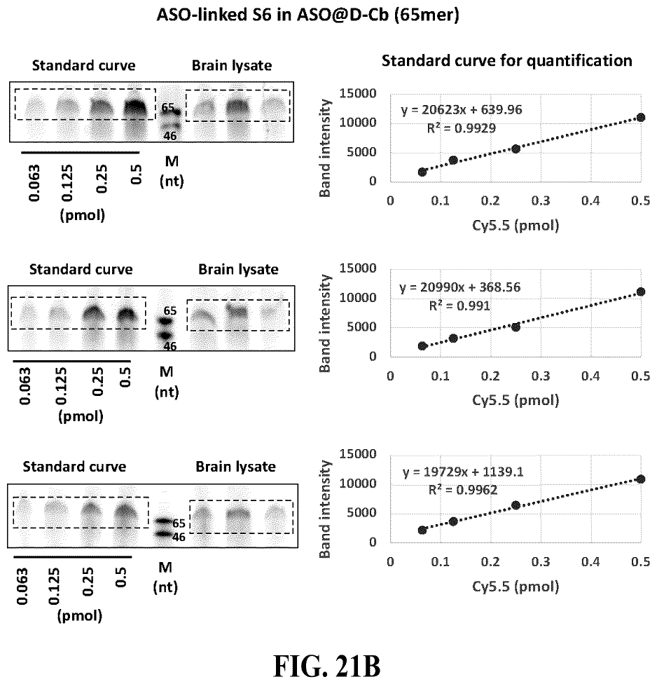

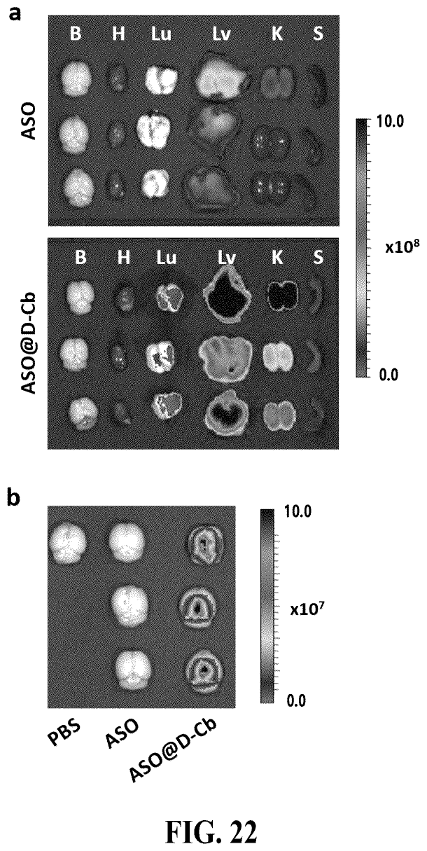

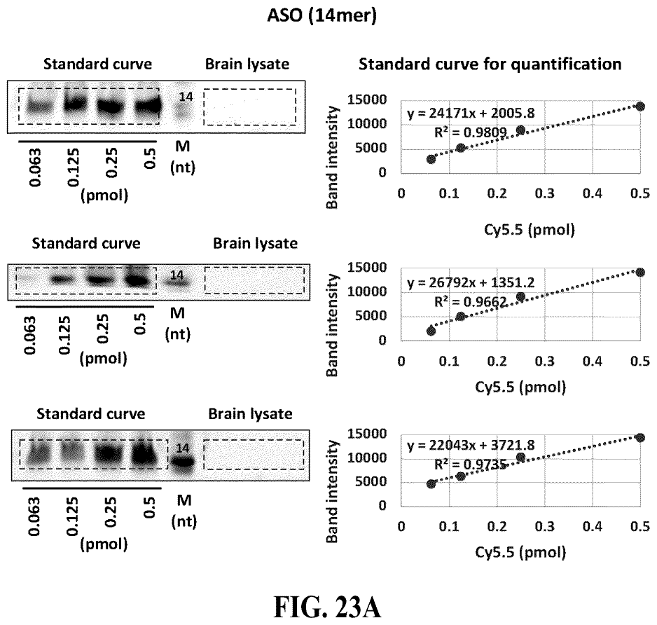

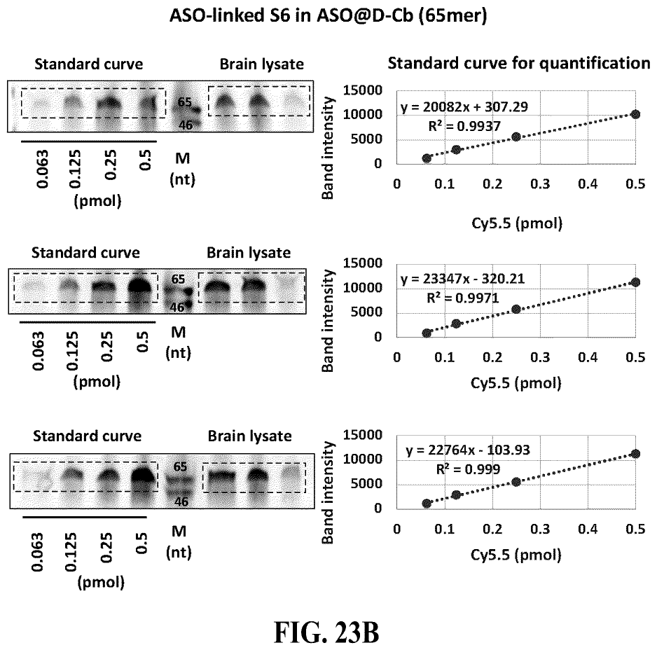

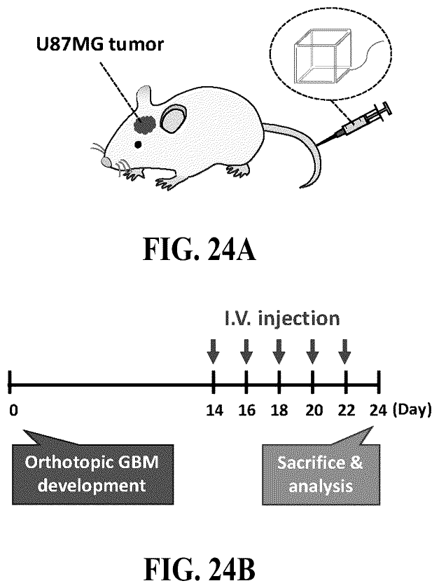

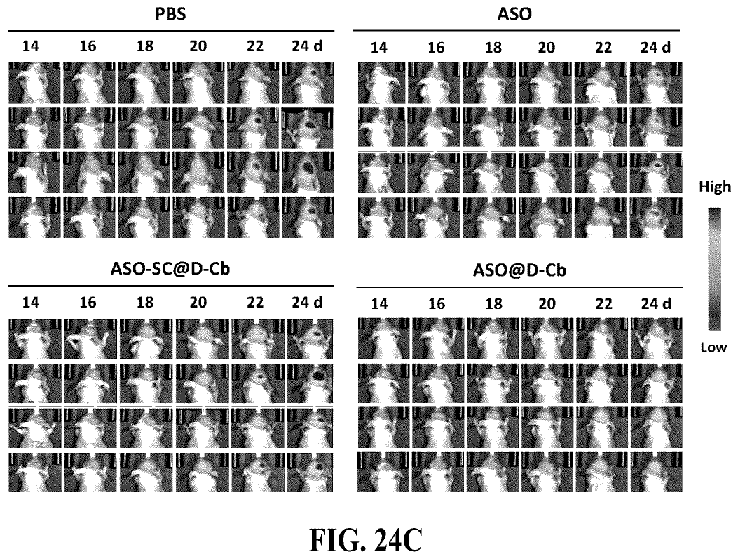

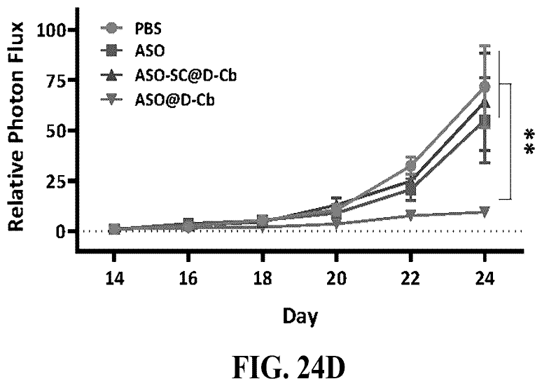

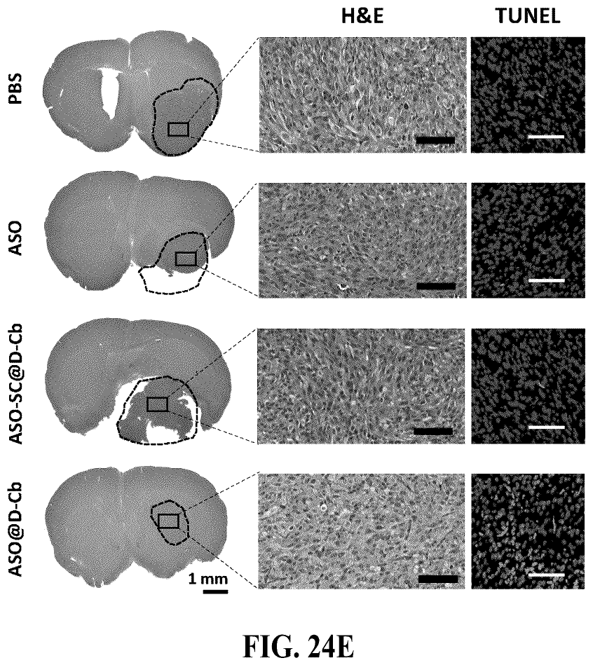

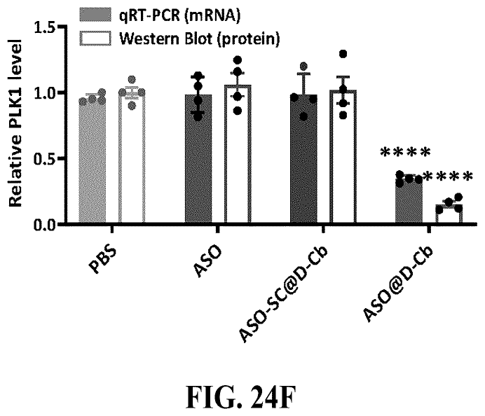

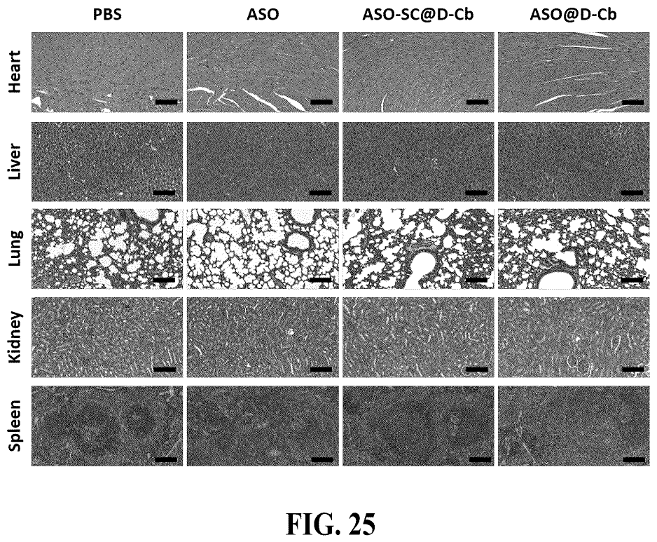

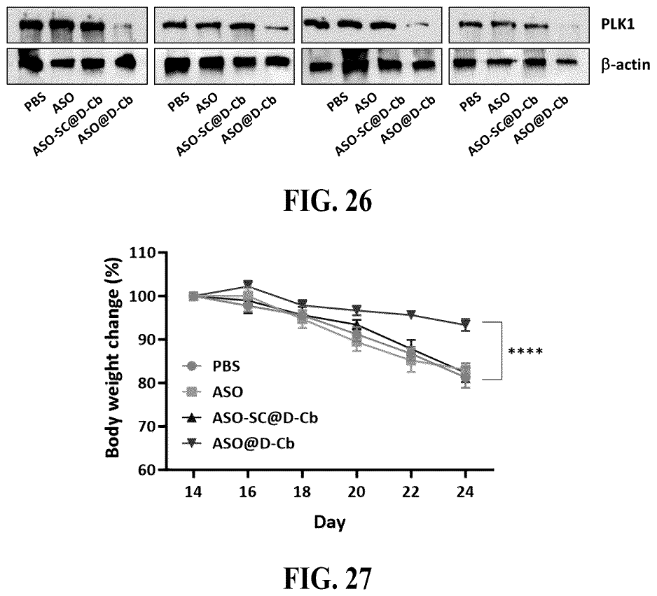

The DNA nanostructures were fabricated and characterized, and their in vivo distribution in mice was investigated. Mice were intravenously injected with the Cy5.5-labeled DNA nanostructures and monitored for 24 hours (FIGS. 3A to 3C). In vivo imaging showed that all the DNA nanostructures except D-Tp were distributed throughout the body within 1 hour after injection. At 2 hours post-injection, major organs were harvested from the mice and imaged to estimate the distribution level of the nanostructures in each organ using Cy5.5 intensity (FIG. 4). Most of the DNA nanostructures were distributed to the liver and kidneys. Focusing on brain distribution, D-Cb showed the highest distribution level, followed by L-Cb and L-Tp (FIG. 3A). The brain distribution level of D-Cb was about 0.25% ID/g as quantified by measuring the Cy5.5 intensity of brain lysates (FIG. 3B). Since Cy5.5 intensity reports the sum of intact and fragmented structures, the inventors also attempted to estimate only the amount of intact D-Cb distributed in the brain by quantifying the band of the intact strand (Cy5.5-S1) in D-Cb based on a denaturing PAGE analysis of the homogenized brain tissue lysates (FIG. 6). The brain distribution level of intact D-Cb was 0.22% ID/g at 2 hours post injection, which was approximately 4-fold higher than that of intact L-Cb although D-Cb is less stable in serum than L-Cb (FIG. 5B and FIG. 2D). Even when the DNA nanostructures were labeled with a negatively charged dye (fluorescein, FAM) instead of a positively charged dye (Cy5.5), their brain distribution levels were not changed significantly (FIG. 5B). This indicates that the brain distribution property of D-Cb was not driven by the fluorescence label. When biotinylated D-Cb was injected, the brain distribution of D-Cb could also be detected by mass analysis of the biotinylated strand (S5) in D-Cb, which was pulled down with streptavidin-coated magnetic beads from brain lysates (FIG. 7). Interestingly, the brain distribution level of D-Cb, even without brain-targeting ligands, was comparable to that of some of the other nanoparticles conjugated with brain-targeting ligands such as ANG, bovine serum albumin (BSA), and transferrin. This demonstrates that the protein corona-assisted brain delivery may similarly be efficient as a targeted delivery system? based on ligand conjugation.

To investigate whether the brain-reaching property of D-Cb is based on BBB penetration, the inventors estimated the BBB-penetration potential of D-Cb using an in vitro BBB monolayer model (FIG. 5C). FAM-labeled D-Cb was added to the apical side of the in vitro BBB model. The amount of the transcytosed DNA nanostructures was estimated by measuring FAM intensity in the basolateral side (FIG. 5C and FIG. 8A). The transcytosis efficiency of D-Cb increased with the incubation time (FIG. 5D). L-Cb showed lower transcytosis efficiency than D-Cb of other shapes with the same cube structure or the same D-backbone (D-Td and D-Tp) (FIG. 5D), indicating that the proper combination of the backbone and the shape is critical for passing through the BBB. Transepithelial/transendothelial electrical resistance (TEER) was not changed after treatment with the DNA nanostructures, indicating that the integrity of the tight junction was maintained during the transcytosis and was not disrupted by the treatment (FIG. 8B). The apparent permeability coefficient (Pₐₚₚ) of D-Cb was also higher than that of any structures sharing either the backbone or the shape with D-Cb, consistently suggesting the potential of the D-Cb structure as a means for penetrating the BBB for brain delivery (FIG. 8C). Unassembled single-stranded DNA strands were hardly able to penetrate the monolayer model, indicating that the three-dimensional D-Cb structure is critical for transcytosis (FIG. 3D and FIG. 8A).

### 3. Proteomic Analysis of the Protein Corona in Cube-Shaped DNA Nanostructures

After observing significant levels of D-Cb distribution within the brain, the inventors intended to identify the factors that contribute to the distribution of D-Cb within the brain. The serum half-life of D-Cb, exceeding 2 hours, may indicate that a significant quantity of D-Cb was distributed within the brain. However, considering that L-Cb, which is much more stable than D-Cb in serum, showed a lower level of brain distribution, the inventors believe that the main factors affecting the brain distribution of D-Cb was something other than the serum half-life. Recent studies have demonstrated the critical role of the protein corona in the biodistribution of DNA nanostructures. Based on these findings, the inventors hypothesized that the formation of a protein corona on D-Cb following intravenous injection may contribute to its distribution within the brain. To investigate the formation of a protein corona by serum proteins, the inventors used mass spectrometry (MS)-based proteomic analysis to identify the proteins in mouse serum that could form a corona on D-Cb (FIG. 9A). Sodium dodecyl sulfate (SDS)-PAGE analysis revealed significantly higher protein adsorption on D-Cb compared to L-Cb (FIG. 9B). The amount of proteins pulled down with L-Cb was similar to the background level observed with naked magnetic beads. Mass spectral analysis identified 210 proteins bound to the three types of samples, with two or more unique peptides assigned. Of these, 157, 179, and 152 proteins were identified in samples pulled down with beads, D-Cb, and L-Cb, respectively, with 114 shared proteins found among the three types of samples (FIG. 9C and Table 4). Label-free quantitative analysis based on peak areas among the three sample groups revealed 165 statistically significant proteins (p-value <0.05) using one-way analysis of variance (ANOVA) from a comparison of the log₂ (normalized abundance) values (FIG. 9D and Table 5).

TABLE 4

| | | | | | # | | | | | | |
|---|---|---|---|---|---|---|---|---|---|---|---|
| Accession | Protein | Gene | Coverage | # | Unique | # | MWc | calc. | | | |
| No. | description | symbol | [%] | PSMs^{a} | Peptides | AAs^{b} | [kDa] | pI | Bead | D-Cb | L-Cb |
| P63101 | 14-3-3 protein zeta/delta | YWHAZ | 20 | 79 | 4 | 245 | 27.8 | 4.79 | ◯ | ◯ | ◯ |
| P68134 | Actin, alpha skeletal muscle | ACTA1 | 55 | 306 | 7 | 377 | 42 | 5.39 | ◯ | ◯ | ◯ |
| P60710 | Actin, cytoplasmic 1 | ACTB | 61 | 417 | 6 | 375 | 41.7 | 5.48 | ◯ | ◯ | ◯ |
| Q60994 | Adiponectin | ADIPOQ | 26 | 69 | 6 | 247 | 26.8 | 5.57 | ◯ | ◯ | ◯ |
| O89020 | Afamin | AFM | 45 | 216 | 24 | 608 | 69.3 | 5.78 | ◯ | ◯ | ◯ |
| P07724 | Albumin | ALB | 90 | 13037 | 78 | 608 | 68.6 | 6.07 | ◯ | ◯ | ◯ |
| Q60590 | Alpha-1-acid | ORM1 | 23 | 58 | 5 | 207 | 23.9 | 5.85 | ◯ | ◯ | ◯ |
| | glycoprotein 1 | | | | | | | | | | |
| P22599 | Alpha-1-antitrypsin 1- 2 | SERPINA1B | 56 | 1071 | 7 | 413 | 45.9 | 5.54 | ◯ | ◯ | ◯ |
| Q00896 | Alpha-1-antitrypsin 1- 3 | SERPINA1C | 52 | 1463 | 5 | 412 | 45.8 | 5.44 | ◯ | ◯ | ◯ |
| Q00897 | Alpha-1-antitrypsin 1- 4 | SERPINA1D | 49 | 835 | 4 | 413 | 46 | 5.44 | ◯ | ◯ | ◯ |
| Q00898 | Alpha-1-antitrypsin 1- 5 | SERPINA1E | 29 | 537 | 3 | 413 | 45.9 | 5.73 | ◯ | ◯ | ◯ |
| Q61247 | Alpha-2-antiplasmin | SERPINF2 | 25 | 187 | 9 | 491 | 54.9 | 6.3 | ◯ | ◯ | ◯ |
| P29699 | Alpha-2-HS- glycoprotein | AHSG | 46 | 575 | 9 | 345 | 37.3 | 6.51 | ◯ | ◯ | ◯ |
| P00687 | Alpha-amylase 1 | AMY1 | 18 | 102 | 7 | 511 | 57.6 | 6.96 | ◯ | ◯ | ◯ |
| P10107 | Annexin A1 | ANXA1 | 16 | 61 | 7 | 346 | 38.7 | 7.37 | ◯ | ◯ | ◯ |
| P07356 | Annexin A2 | ANXA2 | 33 | 48 | 10 | 339 | 38.7 | 7.69 | ◯ | ◯ | ◯ |
| P32261 | Antithrombin-III | SERPINC1 | 47 | 315 | 15 | 465 | 52 | 6.46 | ◯ | ◯ | ◯ |
| Q00623 | Apolipoprotein A-I | APOA1 | 52 | 1024 | 16 | 264 | 30.6 | 5.73 | ◯ | ◯ | ◯ |
| P06728 | Apolipoprotein A-IV | APOA4 | 63 | 351 | 19 | 395 | 45 | 5.47 | ◯ | ◯ | ◯ |
| P08226 | Apolipoprotein E | APOE | 34 | 53 | 9 | 311 | 35.8 | 5.68 | ◯ | ◯ | ◯ |
| Q01339 | Beta-2-glycoprotein 1 | APOH | 44 | 194 | 15 | 345 | 38.6 | 8.22 | ◯ | ◯ | ◯ |
| P08607 | C4b-binding protein | C4BPA | 25 | 74 | 9 | 469 | 51.5 | 7.15 | ◯ | ◯ | ◯ |
| P23953 | Carboxylesterase 1C | CES1C | 38 | 295 | 15 | 554 | 61 | 5.06 | ◯ | ◯ | ◯ |
| Q9DBB9 | Carboxypeptidase | CPN2 | 22 | 40 | 8 | 547 | 60.4 | 5.88 | ◯ | ◯ | ◯ |
| | N subunit 2 | | | | | | | | | | |
| Q9QWK4 | CD5 antigen-like | CD5L | 29 | 75 | 9 | 352 | 38.8 | 5.16 | ◯ | ◯ | ◯ |
| Q61147 | Ceruloplasmin | CP | 53 | 1046 | 48 | 1061 | 121.1 | 5.85 | ◯ | ◯ | ◯ |
| Q06890 | Clusterin | CLU | 16 | 148 | 7 | 448 | 51.6 | 5.67 | ◯ | ◯ | ◯ |
| O88947 | Coagulation factor X | F10 | 22 | 25 | 6 | 481 | 54 | 5.66 | ◯ | ◯ | ◯ |
| P14106 | Complement | C1QB | 27 | 87 | 5 | 253 | 26.7 | 8.15 | ◯ | ◯ | ◯ |
| | C1q subcomponent | | | | | | | | | | |
| | subunit B | | | | | | | | | | |
| Q8CG16 | Complement C1r-A | C1RA | 7 | 22 | 4 | 707 | 80 | 5.66 | ◯ | ◯ | ◯ |
| | subcomponent | | | | | | | | | | |
| P01027 | Complement C3 | C3 | 71 | 2134 | 91 | 1663 | 186.4 | 6.73 | ◯ | ◯ | ◯ |
| P01029 | Complement C4-B | C4B | 39 | 264 | 45 | 1738 | 192.8 | 7.53 | ◯ | ◯ | ◯ |
| P06684 | Complement C5 | C5 | 29 | 265 | 37 | 1680 | 188.8 | 6.81 | ◯ | ◯ | ◯ |
| P04186 | Complement factor B | CFB | 31 | 197 | 23 | 761 | 85 | 7.37 | ◯ | ◯ | ◯ |
| P06909 | Complement factor H | CFH | 44 | 388 | 41 | 1234 | 139 | 6.99 | ◯ | ◯ | ◯ |
| Q61129 | Complement factor I | CFI | 23 | 56 | 9 | 603 | 67.2 | 7.46 | ◯ | ◯ | ◯ |
| Q06770 | Corticosteroid-binding | SERPINA6 | 16 | 44 | 6 | 397 | 44.7 | 5.24 | ◯ | ◯ | ◯ |
| | globulin | | | | | | | | | | |
| P10126 | Elongation | EEF1A1 | 21 | 55 | 8 | 462 | 50.1 | 9.01 | ◯ | ◯ | ◯ |
| | Factor 1-alpha 1 | | | | | | | | | | |
| P20029 | Endoplasmic | HSPA5 | 9 | 33 | 3 | 655 | 72.4 | 5.16 | ◯ | ◯ | ◯ |
| | reticulum | | | | | | | | | | |
| | chaperone BiP | | | | | | | | | | |
| Q01279 | Epidermal growth | EGFR | 11 | 56 | 10 | 1210 | 134.8 | 6.86 | ◯ | ◯ | ◯ |
| | factor receptor | | | | | | | | | | |
| Q9QXC1 | Fetuin-B | FETUB | 38 | 30 | 8 | 388 | 42.7 | 6.61 | ◯ | ◯ | ◯ |
| P11276 | Fibronectin | FN1 | 26 | 324 | 44 | 2477 | 272.4 | 5.59 | ◯ | ◯ | ◯ |
| P05064 | Fructose- | ALDOA | 24 | 27 | 7 | 364 | 39.3 | 8.09 | ◯ | ◯ | ◯ |
| | bisphosphate | | | | | | | | | | |
| | aldolase A | | | | | | | | | | |
| P13020 | Gelsolin | GSN | 38 | 298 | 23 | 780 | 85.9 | 6.18 | ◯ | ◯ | ◯ |
| P01898 | H-2 class I | H2-Q10 | 46 | 141 | 11 | 325 | 37.2 | 5.25 | ◯ | ◯ | ◯ |
| | histocompatibility | | | | | | | | | | |
| | antigen, Q10 | | | | | | | | | | |
| | alpha chain | | | | | | | | | | |
| Q61646 | Haptoglobin | HP | 59 | 495 | 18 | 347 | 38.7 | 6.29 | ◯ | ◯ | ◯ |
| P63017 | Heat shock cognate 71 kDa | HSPA8 | 10 | 32 | 4 | 646 | 70.8 | 5.52 | ◯ | ◯ | ◯ |
| | protein | | | | | | | | | | |
| P01942 | Hemoglobin subunit | HBA | 84 | 741 | 9 | 142 | 15.1 | 8.22 | ◯ | ◯ | ◯ |
| | alpha | | | | | | | | | | |
| P02088 | Hemoglobin subunit | HBB-B1 | 88 | 743 | 6 | 147 | 15.8 | 7.65 | ◯ | ◯ | ◯ |
| | beta-1 | | | | | | | | | | |
| Q91X72 | Hemopexin | HPX | 66 | 1889 | 32 | 460 | 51.3 | 7.8 | ◯ | ◯ | ◯ |
| P49182 | Heparin cofactor 2 | SERPIND1 | 23 | 60 | 8 | 478 | 54.5 | 7.34 | ◯ | ◯ | ◯ |
| Q9ESB3 | Histidine-rich | HRG | 28 | 313 | 14 | 525 | 59.1 | 7.66 | ◯ | ◯ | ◯ |
| | glycoprotein | | | | | | | | | | |
| Q64475 | Histone H2B type 1-B | H2BC3 | 37 | 13 | 4 | 126 | 13.9 | 10.32 | ◯ | ◯ | ◯ |
| P62806 | Histone H4 | H4F16 | 55 | 94 | 8 | 103 | 11.4 | 11.36 | ◯ | ◯ | ◯ |
| P01878 | Ig alpha chain | | 51 | 354 | 12 | 344 | 36.9 | 5.06 | ◯ | ◯ | ◯ |
| | C region | | | | | | | | | | |
| P01869 | Ig gamma-1 chain | IGHG1 | 44 | 330 | 12 | 393 | 43.4 | 6.44 | ◯ | ◯ | ◯ |
| | C region, | | | | | | | | | | |
| | membrane-bound form | | | | | | | | | | |
| P01864 | Ig gamma-2A chain | | 25 | 513 | 4 | 335 | 36.6 | 8.22 | ◯ | ◯ | ◯ |
| | C region secreted form | | | | | | | | | | |
| P01865 | Ig gamma-2A chain | IGH-1A | 43 | 492 | 7 | 398 | 43.9 | 6.29 | ◯ | ◯ | ◯ |
| | C region, | | | | | | | | | | |
| | membrane-bound form | | | | | | | | | | |
| P03987 | Ig gamma-3 chain | | 60 | 153 | 13 | 398 | 43.9 | 7.14 | ◯ | ◯ | ◯ |
| | C region | | | | | | | | | | |
| P18531 | Ig heavy chain | IGHV3-6 | 31 | 33 | 2 | 116 | 13.1 | 8.78 | ◯ | ◯ | ◯ |
| | V region 3-6 | | | | | | | | | | |
| P18527 | Ig heavy chain | | 47 | 47 | 2 | 97 | 10.7 | 9.17 | ◯ | ◯ | ◯ |
| | V region 914 | | | | | | | | | | |
| P01746 | Ig heavy chain | | 17 | 25 | 2 | 140 | 15.5 | 8.4 | ◯ | ◯ | ◯ |
| | V region 93G7 | | | | | | | | | | |
| P06330 | Ig heavy chain | | 72 | 146 | 3 | 118 | 12.9 | 7.11 | ◯ | ◯ | ◯ |
| | V region AC38 205.12 | | | | | | | | | | |
| P01791 | Ig heavy chain | | 20 | 22 | 2 | 123 | 13.9 | 6.06 | ◯ | ◯ | ◯ |
| | V region HPCM6 | | | | | | | | | | |
| P18524 | Ig heavy chain | | 44 | 38 | 3 | 117 | 12.9 | 19.29 | ◯ | ◯ | ◯ |
| | V region RF | | | | | | | | | | |
| P01807 | Ig heavy chain | | 48 | 82 | 2 | 119 | 13.2 | 7.94 | ◯ | ◯ | ◯ |
| | V region X44 | | | | | | | | | | |
| P01632 | Ig kappa chain | IGKV7-33 | 14 | 17 | 2 | 114 | 12.7 | 8.98 | ◯ | ◯ | ◯ |
| | V-I region S107A | | | | | | | | | | |
| P01631 | Ig kappa chain | | 43 | 41 | 4 | 113 | 12.3 | 8.88 | ◯ | ◯ | ◯ |
| | V-II region 26-10 | | | | | | | | | | |
| P01630 | Ig kappa chain | | 21 | 21 | 2 | 113 | 12.5 | 8.65 | ◯ | ◯ | ◯ |
| | V-II region 7S34.1 | | | | | | | | | | |
| P01638 | Ig kappa chain | | 36 | 81 | 3 | 115 | 13 | 7.81 | ◯ | ◯ | ◯ |
| | V-V region L6 | | | | | | | | | | |
| | (Fragment) | | | | | | | | | | |
| P01642 | Ig kappa chain | GM10881 | 23 | 12 | 2 | 115 | 12.6 | 5.94 | ◯ | ◯ | ◯ |
| | V-V region L7 | | | | | | | | | | |
| | (Fragment) | | | | | | | | | | |
| P01636 | Ig kappa chain | | 35 | 18 | 3 | 108 | 12 | 7.28 | ◯ | ◯ | ◯ |
| | V-V region MOPC 149 | | | | | | | | | | |
| P01843 | Ig lambda-1 chain | | 81 | 212 | 5 | 105 | 11.6 | 6.27 | ◯ | ◯ | ◯ |
| | C region | | | | | | | | | | |
| P01723 | Ig lambda-1 chain | | 34 | 32 | 2 | 117 | 12.2 | 5.21 | ◯ | ◯ | ◯ |
| | V region | | | | | | | | | | |
| P01844 | Ig lambda-2 chain | IGLC2 | 86 | 76 | 5 | 104 | 11.2 | 6.27 | ◯ | ◯ | ◯ |
| | C region | | | | | | | | | | |
| P01728 | Ig lambda-2 chain | | 34 | 22 | 2 | 117 | 12.2 | 5.74 | ◯ | ◯ | ◯ |
| | V region | | | | | | | | | | |
| P01867 | Immunoglobulin heavy | IGHG2B | 45 | 430 | 10 | 404 | 44.2 | 6.52 | ◯ | ◯ | ◯ |
| | constant gamma 2B | | | | | | | | | | |
| P01872 | Immunoglobulin heavy | IGHM | 45 | 454 | 18 | 454 | 49.9 | 7.01 | ◯ | ◯ | ◯ |
| | constant mu | | | | | | | | | | |
| P01635 | Immunoglobulin kappa | IGKV12-41 | 41 | 60 | 4 | 115 | 12.6 | 8.31 | ◯ | ◯ | ◯ |
| | chain variable 12-41 | | | | | | | | | | |
| | (Fragment) | | | | | | | | | | |
| P01633 | Immunoglobulin kappa | IGKV6-17 | 17 | 35 | 3 | 149 | 16.4 | 6.92 | ◯ | ◯ | ◯ |
| | chain variable 6-17 | | | | | | | | | | |
| P01837 | Immunoglobulin kappa | IGKC | 93 | 797 | 11 | 107 | 11.9 | 5.9 | ◯ | ◯ | ◯ |
| | constant | | | | | | | | | | |
| Q9DBD0 | Inhibitor of | ICA | 56 | 220 | 27 | 700 | 76.7 | 7.25 | ◯ | ◯ | ◯ |
| | carbonic anhydrase | | | | | | | | | | |
| A6X935 | Inter alpha-trypsin inhibitor, | ITIH4 | 46 | 381 | 33 | 942 | 104.6 | 6.4 | ◯ | ◯ | ◯ |
| | heavy chain 4 | | | | | | | | | | |
| Q61702 | Inter-alpha-trypsin inhibitor | ITIH1 | 36 | 136 | 22 | 907 | 101 | 6.96 | ◯ | ◯ | ◯ |
| | heavy chain H1 | | | | | | | | | | |
| Q61703 | Inter-alpha-trypsin inhibitor | ITIH2 | 29 | 124 | 20 | 946 | 105.9 | 7.27 | ◯ | ◯ | ◯ |
| | heavy chain H2 | | | | | | | | | | |
| Q61704 | Inter-alpha-trypsin inhibitor | ITIH3 | 26 | 104 | 17 | 889 | 99.3 | 6.05 | ◯ | ◯ | ◯ |
| | heavy chain H3 | | | | | | | | | | |
| O08677 | Kininogen-1 | KNG1 | 25 | 324 | 13 | 661 | 73.1 | 6.54 | ◯ | ◯ | ◯ |
| P28665 | Murinoglobulin-1 | MUG1 | 55 | 1350 | 39 | 1476 | 165.2 | 6.42 | ◯ | ◯ | ◯ |
| P28666 | Murinoglobulin-2 | MUG2 | 47 | 802 | 29 | 1451 | 162.3 | 6.74 | ◯ | ◯ | ◯ |
| O70362 | Phosphatidylinositol- | GPLD1 | 18 | 53 | 13 | 837 | 93.2 | 7.12 | ◯ | ◯ | ◯ |
| | glycan-specific | | | | | | | | | | |
| | phospholipase D | | | | | | | | | | |
| P09411 | Phosphoglycerate | PGK1 | 10 | 16 | 3 | 417 | 44.5 | 7.9 | ◯ | ◯ | ◯ |
| | kinase 1 | | | | | | | | | | |
| Q9DBJ1 | Phosphoglycerate | PGAM1 | 14 | 16 | 3 | 254 | 28.8 | 7.18 | ◯ | ◯ | ◯ |
| | mutase 1 | | | | | | | | | | |
| P26262 | Plasma kallikrein | KLKB1 | 26 | 93 | 13 | 638 | 71.3 | 8.02 | ◯ | ◯ | ◯ |
| P97290 | Plasma protease C1 | SERPING1 | 41 | 129 | 16 | 504 | 55.5 | 6.29 | ◯ | ◯ | ◯ |
| | inhibitor | | | | | | | | | | |
| P20918 | Plasminogen | PLG | 72 | 543 | 47 | 812 | 90.7 | 6.6 | ◯ | ◯ | ◯ |
| Q61838 | Pregnancy zone protein | PZP | 61 | 2568 | 71 | 1495 | 165.7 | 6.68 | ◯ | ◯ | ◯ |
| P11680 | Properdin | CFP | 14 | 68 | 4 | 464 | 50.3 | 7.84 | ◯ | ◯ | ◯ |
| Q07456 | Protein AMBP | AMBP | 19 | 57 | 6 | 349 | 39 | 6.32 | ◯ | ◯ | ◯ |
| Q9D2Q8 | Protein S100-A14 | S100A14 | 21 | 12 | 2 | 104 | 11.6 | 5.5 | ◯ | ◯ | ◯ |
| P19221 | Prothrombin | F2 | 31 | 170 | 16 | 618 | 70.2 | 6.43 | ◯ | ◯ | ◯ |
| Q00724 | Retinol-binding protein 4 | RBP4 | 34 | 23 | 5 | 201 | 23.2 | 5.99 | ◯ | ◯ | ◯ |
| P07759 | Serine protease | SERPINA3K | 61 | 1041 | 16 | 418 | 46.9 | 5.16 | ◯ | ◯ | ◯ |
| | inhibitor A3K | | | | | | | | | | |
| Q921I1 | Serotransferrin | TF | 73 | 5294 | 65 | 697 | 76.7 | 7.18 | ◯ | ◯ | ◯ |
| P12246 | Serum amyloid P- | APCS | 27 | 104 | 6 | 224 | 26.2 | 6.35 | ◯ | ◯ | ◯ |
| | component | | | | | | | | | | |
| P52430 | Serum paraoxonase/ | PON1 | 34 | 109 | 8 | 355 | 39.5 | 5.22 | ◯ | ◯ | ◯ |
| | arylesterase 1 | | | | | | | | | | |
| Q8BND5 | Sulfhydryl oxidase 1 | QSOX1 | 18 | 60 | 11 | 748 | 82.7 | 7.17 | ◯ | ◯ | ◯ |
| P35441 | Thrombospondin-1 | THBS1 | 26 | 176 | 23 | 1170 | 129.6 | 4.96 | ◯ | ◯ | ◯ |
| P07309 | Transthyretin | TTR | 67 | 449 | 8 | 147 | 15.8 | 6.16 | ◯ | ◯ | ◯ |
| P17751 | Triosephosphate isomerase | TPI1 | 30 | 17 | 6 | 249 | 26.7 | 7.3 | ◯ | ◯ | ◯ |
| P68368 | Tubulin alpha-4A chain | TUBA4A | 11 | 18 | 4 | 448 | 49.9 | 5.06 | ◯ | ◯ | ◯ |
| P20152 | Vimentin | VIM | 26 | 190 | 9 | 466 | 53.7 | 5.12 | ◯ | ◯ | ◯ |
| P21614 | Vitamin D-binding protein | GC | 74 | 474 | 26 | 476 | 53.6 | 5.5 | ◯ | ◯ | ◯ |
| P29788 | Vitronectin | VTN | 20 | 98 | 5 | 478 | 54.8 | 5.88 | ◯ | ◯ | ◯ |
| Q64726 | Zinc-alpha-2- glycoprotein | AZGP1 | 35 | 69 | 8 | 307 | 35.3 | 6.18 | ◯ | ◯ | ◯ |
| P07361 | Alpha-1-acid glycoprotein 2 | ORM2 | 15 | 33 | 2 | 207 | 23.8 | 5.45 | X | ◯ | ◯ |
| P11859 | Angiotensinogen | AGT | 22 | 28 | 8 | 477 | 52 | 5.44 | X | ◯ | ◯ |
| E9Q414 | Apolipoprotein B-100 | APOB | 9 | 103 | 32 | 4505 | 509.1 | 6.81 | X | ◯ | ◯ |
| P01887 | Beta-2-microglobulin | B2M | 16 | 15 | 2 | 119 | 13.8 | 8.44 | X | ◯ | ◯ |
| Q9JJN5 | Carboxypeptidase | CPN1 | 22 | 37 | 7 | 457 | 51.8 | 8.28 | X | ◯ | ◯ |
| | N catalytic chain | | | | | | | | | | |
| O88783 | Coagulation factor V | F5 | 2 | 26 | 4 | 2183 | 247.1 | 6.05 | X | ◯ | ◯ |
| Q07968 | Coagulation factor XIII B | F13B | 4 | 15 | 3 | 669 | 76.1 | 6.92 | X | ◯ | ◯ |
| | chain | | | | | | | | | | |
| P06683 | Complement component C9 | C9 | 8 | 14 | 3 | 548 | 62 | 5.78 | X | ◯ | ◯ |
| P01749 | Ig heavy chain | IGHV1-61 | 28 | 66 | 2 | 117 | 13 | 7.87 | X | ◯ | ◯ |
| | V region 3 | | | | | | | | | | |
| P01801 | Ig heavy chain | | 43 | 196 | 3 | 115 | 12.8 | 7.12 | X | ◯ | ◯ |
| | V-III region J606 | | | | | | | | | | |
| P84750 | Ig kappa chain | | 16 | 3 | 2 | 121 | 13.2 | 8.81 | X | ◯ | ◯ |
| | V region Mem5 | | | | | | | | | | |
| | (Fragment) | | | | | | | | | | |
| P01660 | Ig kappa chain | | 49 | 14 | 2 | 111 | 12.1 | 5.38 | X | ◯ | ◯ |
| | V-III region | | | | | | | | | | |
| | PC 3741/TEPC 111 | | | | | | | | | | |
| P08071 | Lactotransferrin | LTF | 3 | 11 | 2 | 707 | 77.8 | 8.53 | X | ◯ | ◯ |
| P42703 | Leukemia inhibitory factor | LIFR | 8 | 16 | 6 | 1092 | 122.5 | 6.04 | X | ◯ | ◯ |
| | receptor | | | | | | | | | | |
| P26041 | Moesin | MSN | 5 | 4 | 2 | 577 | 67.7 | 6.6 | X | ◯ | ◯ |
| Q8VCS0 | N-acetylmuramoyl-L- | PGLYRP2 | 6 | 19 | 3 | 530 | 57.7 | 6.98 | X | ◯ | ◯ |
| | alanine amidase | | | | | | | | | | |
| P97298 | Pigment epithelium- | SERPINF1 | 27 | 17 | 8 | 417 | 46.2 | 6.98 | X | ◯ | ◯ |
| | derived factor | | | | | | | | | | |
| Q61233 | Plastin-2 | LCP1 | 3 | 16 | 2 | 627 | 70.1 | 5.33 | X | ◯ | ◯ |
| P31532 | Serum amyloid A-4 protein | SAA4 | 42 | 23 | 6 | 130 | 15.1 | 9.26 | X | ◯ | ◯ |
| Q62351 | Transferrin receptor | TFRC | 13 | 19 | 10 | 763 | 85.7 | 6.57 | X | ◯ | ◯ |
| | protein 1 | | | | | | | | | | |
| O70456 | 14-3-3 protein sigma | SFN | 17 | 19 | 4 | 248 | 27.7 | 4.78 | ◯ | X | ◯ |
| P17182 | Alpha-enolase | ENO1 | 6 | 3 | 2 | 434 | 47.1 | 6.8 | ◯ | X | ◯ |
| E9Q557 | Desmoplakin | DSP | 8 | 78 | 18 | 2883 | 332.7 | 6.8 | ◯ | X | ◯ |
| Q8VCM7 | Fibrinogen gamma chain | FGG | 7 | 12 | 2 | 436 | 49.4 | 5.86 | ◯ | X | ◯ |
| P16858 | Glyceraldehyde-3- | GAPDH | 29 | 20 | 6 | 333 | 35.8 | 8.25 | ◯ | X | ◯ |
| | phosphate dehydrogenase | | | | | | | | | | |
| Q61696 | Heat shock 70 kDa | HSPA1A | 9 | 21 | 4 | 641 | 70 | 5.72 | ◯ | X | ◯ |
| | protein 1A | | | | | | | | | | |
| P07901 | Heat shock protein | HSP90AA1 | 1 | 9 | 2 | 733 | 84.7 | 5.01 | ◯ | X | ◯ |
| | HSP 90-alpha | | | | | | | | | | |
| P11499 | Heat shock protein | HSP90AB1 | 8 | 14 | 4 | 724 | 83.2 | 5.03 | ◯ | X | ◯ |
| | HSP 90-beta | | | | | | | | | | |
| Q02257 | Junction plakoglobin | JUP | 10 | 21 | 6 | 745 | 81.7 | 6.14 | ◯ | X | ◯ |
| P11247 | Myeloperoxidase | MPO | 5 | 12 | 3 | 718 | 81.1 | 9.55 | ◯ | X | ◯ |
| Q8VDD5 | Myosin-9 | MYH9 | 2 | 6 | 3 | 1960 | 226.2 | 5.66 | ◯ | X | ◯ |
| P17742 | Peptidyl-prolyl | PPIA | 32 | 15 | 4 | 164 | 18 | 7.9 | ◯ | X | ◯ |
| | cis-trans isomerase A | | | | | | | | | | |
| P97350 | Plakophilin-1 | PKP1 | 6 | 24 | 4 | 728 | 80.8 | 8.91 | ◯ | X | ◯ |
| P48678 | Prelamin-A/C | LMNA | 6 | 14 | 3 | 665 | 74.2 | 6.98 | ◯ | X | ◯ |
| P09103 | Protein disulfide- isomerase | P4HB | 7 | 15 | 3 | 509 | 57 | 4.88 | ◯ | X | ◯ |
| P68372 | Tubulin beta-4B chain | TUBB4B | 21 | 43 | 2 | 445 | 49.8 | 4.89 | ◯ | X | ◯ |
| Q60930 | Voltage-dependent | VDAC2 | 11 | 3 | 2 | 295 | 31.7 | 7.49 | ◯ | X | ◯ |
| | anion-selective channel | | | | | | | | | | |
| | protein 2 | | | | | | | | | | |
| Q9R269 | Periplakin | PPL | 1 | 2 | 2 | 1755 | 203.9 | 5.54 | X | X | ◯ |
| P98086 | Complement C1q | C1QA | 16 | 29 | 3 | 245 | 26 | 9.11 | ◯ | ◯ | X |
| | subcomponent | | | | | | | | | | |
| | subunit A | | | | | | | | | | |
| Q02105 | Complement C1q | C1QC | 16 | 140 | 3 | 246 | 26 | 8.54 | ◯ | ◯ | X |
| | subcomponent | | | | | | | | | | |
| | subunit C | C8A | | | | | | | | | |
| Q8K182 | Complement component | | 7 | 9 | 3 | 587 | 66 | 6.54 | ◯ | ◯ | X |
| | C8 alpha chain | | | | | | | | | | |
| Q8BH35 | Complement component | C8B | 16 | 23 | 6 | 589 | 66.2 | 7.77 | ◯ | ◯ | X |
| | C8 beta chain | | | | | | | | | | |
| P62897 | Cytochrome c, | CYCS | 24 | 9 | 2 | 105 | 11.6 | 9.58 | ◯ | ◯ | X |
| | somatic | | | | | | | | | | |
| Q08879 | Fibulin-1 | FBLN1 | 5 | 7 | 2 | 705 | 78 | 5.16 | ◯ | ◯ | X |
| Q9R098 | Hepatocyte growth factor | HGFAC | 6 | 53 | 3 | 653 | 70.5 | 7.03 | ◯ | ◯ | X |
| | activator | | | | | | | | | | |
| P06151 | L-lactate dehydrogenase A | LDHA | 16 | 38 | 4 | 332 | 36.5 | 7.74 | ◯ | ◯ | X |
| | chain | | | | | | | | | | |
| O70570 | Polymeric immunoglobulin | PIGR | 5 | 12 | 2 | 771 | 84.9 | 5.4 | ◯ | ◯ | X |
| | receptor | | | | | | | | | | |
| Q8R121 | Protein Z-dependent | SERPINA10 | 17 | 27 | 6 | 448 | 51.8 | 5.67 | ◯ | ◯ | X |
| | protease inhibitor | | | | | | | | | | |
| Q91WP6 | Serine protease | SERPINA3N | 20 | 251 | 5 | 418 | 46.7 | 5.82 | ◯ | ◯ | X |
| | inhibitor A3N | | | | | | | | | | |
| P61939 | Thyroxine-binding globulin | SERPINA7 | 11 | 28 | 4 | 418 | 47 | 6.54 | ◯ | ◯ | X |
| Q9QZ25 | Vascular non-inflammatory | VNN3 | 7 | 6 | 3 | 500 | 56.3 | 6.29 | ◯ | ◯ | X |
| | molecule 3 | | | | | | | | | | |
| Q6GQT1 | Alpha-2- macroglobulin-P | A2M | 3 | 36 | 2 | 1474 | 164.2 | 6.61 | X | ◯ | X |
| Q9Z1R3 | Apolipoprotein M | APOM | 7 | 5 | 2 | 190 | 21.3 | 6.52 | X | ◯ | X |
| P00920 | Carbonic anhydrase 2 | CA2 | 18 | 16 | 4 | 260 | 29 | 7.01 | X | ◯ | X |
| Q9JHH6 | Carboxypeptidase B2 | CPB2 | 13 | 20 | 4 | 422 | 48.8 | 7.97 | X | ◯ | X |
| P10605 | Cathepsin B | CTSB | 9 | 3 | 2 | 339 | 37.3 | 5.91 | X | ◯ | X |
| Q03311 | Cholinesterase | BCHE | 4 | 2 | 2 | 603 | 68.4 | 7.25 | X | ◯ | X |
| Q80YC5 | Coagulation factor XII | F12 | 16 | 29 | 6 | 597 | 65.7 | 6.84 | X | ◯ | X |
| Q8CG14 | Complement C1s-1 | C1S1 | 14 | 20 | 7 | 688 | 76.8 | 5.08 | X | ◯ | X |
| | subcomponent | | | | | | | | | | |
| P21180 | Complement C2 | C2 | 8 | 25 | 5 | 760 | 84.7 | 7.56 | X | ◯ | X |
| Q8VCG4 | Complement component | C8G | 34 | 26 | 5 | 202 | 22.5 | 9.25 | X | ◯ | X |
| | C8 gamma chain | | | | | | | | | | |
| P14847 | C-reactive protein | CRP | 17 | 28 | 3 | 225 | 25.3 | 6.2 | X | ◯ | X |
| Q8BPB5 | EGF-containing fibulin-like | EFEMP1 | 6 | 6 | 2 | 493 | 54.9 | 5.14 | X | ◯ | X |
| | extracellular matrix protein 1 | | | | | | | | | | |
| Q8K1B8 | Fermitin family | FERMT3 | 6 | 12 | 3 | 665 | 75.6 | 7.05 | X | ◯ | X |
| | homolog 3 | | | | | | | | | | |
| Q8BTM8 | Filamin-A | FLNA | 1 | 11 | 2 | 2647 | 281 | 6.04 | X | ◯ | X |
| Q923D2 | Flavin reductase | BLVRB | 13 | 5 | 2 | 206 | 22.2 | 7.01 | X | ◯ | X |
| | (NADPH) | | | | | | | | | | |
| Q91Y97 | Fructose-bisphosphate | ALDOB | 9 | 14 | 2 | 364 | 39.5 | 8.27 | X | ◯ | X |
| | aldolase B | | | | | | | | | | |
| P46412 | Glutathione peroxidase 3 | GPX3 | 29 | 21 | 5 | 226 | 25.4 | 8.22 | X | ◯ | X |
| P14426 | H-2 class I histocompatibility | H2-D1 | 11 | 10 | 3 | 362 | 40.6 | 5.47 | X | ◯ | X |
| | antigen, | | | | | | | | | | |
| | D-K alpha chain | | | | | | | | | | |
| P02089 | Hemoglobin subunit beta-2 | HBB-B2 | 62 | 343 | 3 | 147 | 15.9 | 8.05 | X | ◯ | X |
| P06336 | Ig epsilon chain C region | | 15 | 49 | 5 | 421 | 47.3 | 7.44 | X | ◯ | X |
| P01670 | Ig kappa chain | | 60 | 44 | 2 | 111 | 12 | 8 | X | ◯ | X |
| | V-III region PC 6684 | | | | | | | | | | |
| P70389 | Insulin-like growth factor- | IGFALS | 22 | 36 | 11 | 603 | 66.9 | 6.6 | X | ◯ | X |
| | binding protein complex | | | | | | | | | | |
| | acid labile subunit | | | | | | | | | | |
| P09581 | Macrophage colony-stimulating | CSF1R | 2 | 8 | 2 | 977 | 109.1 | 6.21 | X | ◯ | X |
| | factor 1 receptor | | | | | | | | | | |
| P16301 | Phosphatidylcholine-sterol | LCAT | 5 | 2 | 2 | 438 | 49.7 | 6.43 | X | ◯ | X |
| | acyltransferase | | | | | | | | | | |
| Q9Z126 | Platelet factor 4 | PF4 | 29 | 15 | 3 | 105 | 11.2 | 9.29 | X | ◯ | X |
| O08742 | Platelet glycoprotein V | GP5 | 8 | 16 | 3 | 567 | 63.4 | 8.97 | X | ◯ | X |
| Q60963 | Platelet-activating | PLA2G7 | 6 | 23 | 2 | 440 | 49.2 | 7.12 | X | ◯ | X |
| | factor acetylhydrolase | | | | | | | | | | |
| P70274 | Selenoprotein P | SELENOP | 12 | 21 | 3 | 380 | 42.7 | 7.09 | X | ◯ | X |
| Q03734 | Serine protease | SERPINA3M | 36 | 423 | 5 | 418 | 47 | 6.1 | X | ◯ | X |
| | inhibitor A3M | | | | | | | | | | |
| P26039 | Talin-1 | TLN1 | 2 | 5 | 4 | 2541 | 269.7 | 6.18 | X | ◯ | X |
| O88968 | Transcobalamin-2 | TCN2 | 16 | 3 | 3 | 430 | 47.6 | 6.33 | X | ◯ | X |
| P82198 | Transforming growth factor- | TGFBI | 3 | 2 | 2 | 683 | 74.6 | 7.06 | X | ◯ | X |
| | beta-induced protein ig-h3 | | | | | | | | | | |
| Q7TPR4 | Alpha-actinin-1 | ACTN1 | 6 | 16 | 2 | 892 | 103 | 5.38 | ◯ | X | X |
| Q9JI91 | Alpha-actinin-2 | ACTN2 | 14 | 13 | 9 | 894 | 103.8 | 5.45 | ◯ | X | X |
| Q9WUA3 | ATP-dependent 6- | PFKP | 2 | 4 | 2 | 784 | 85.4 | 7.11 | ◯ | X | X |
| | phosphofructokinase, | | | | | | | | | | |
| | platelet type | | | | | | | | | | |
| P21550 | Beta-enolase | ENO3 | 5 | 3 | 2 | 434 | 47 | 7.18 | ◯ | X | X |
| P07310 | Creatine kinase M-type | CKM | 11 | 3 | 2 | 381 | 43 | 7.06 | ◯ | X | X |
| Q9WUB3 | Glycogen phosphorylase, | PYGM | 9 | 10 | 6 | 842 | 97.2 | 7.11 | ◯ | X | X |
| | muscle form | | | | | | | | | | |
| P97457 | Myosin regulatory | MYL11 | 17 | 2 | 2 | 169 | 18.9 | 4.92 | ◯ | X | X |
| | light chain 11 | | | | | | | | | | |
| Q5SX40 | Myosin-1 | MYH1 | 22 | 111 | 2 | 1942 | 223.2 | 5.76 | ◯ | X | X |
| P13541 | Myosin-3 | MYH3 | 12 | 61 | 2 | 1940 | 223.7 | 5.81 | ◯ | X | X |
| Q91Z83 | Myosin-7 | MYH7 | 10 | 29 | 2 | 1935 | 222.7 | 5.76 | ◯ | X | X |
| P13542 | Myosin-8 | MYH8 | 20 | 97 | 2 | 1937 | 222.6 | 5.83 | ◯ | X | X |
| A2ASS6 | Titin | TTN | 0 | 10 | 6 | 35213 | 3904.1 | 6.2 | ◯ | X | X |
| P21107 | Tropomyosin alpha-3 chain | TPM3 | 26 | 53 | 2 | 285 | 33 | 4.72 | ◯ | X | X |

TABLE 5

| | | | Normalized | | | Normalized | | |
|---|---|---|---|---|---|---|---|---|
| Accession | Protein | Gene | abundance_bead | | | abundance_D-Cb | | |
| No. | description | symbol | 1 | 2 | 3 | 1 | 2 | 3 |
| P19221 | Prothrombin | F2 | 2.64E+07 | 2.18E+07 | 2.29E+07 | 1.27E+08 | 1.63E+08 | 1.52E+08 |
| E9Q557 | Desmoplakin | DSP | 6.10E+06 | 7.98E+06 | 1.14E+07 | 8.18E+04 | 2.48E+05 | 2.01E+05 |
| P06684 | Complement C5 | C5 | 1.07E+07 | 8.69E+06 | 9.72E+06 | 1.26E+08 | 2.34E+08 | 1.63E+08 |
| P52430 | Serum | PON1 | 1.35E+07 | 1.60E+07 | 1.49E+07 | 1.85E+08 | 1.12E+08 | 1.87E+08 |
| | paraoxonase/ | | | | | | | |
| | arylesterase 1 | | | | | | | |
| Q9R269 | Periplakin | PPL | 1.82E+05 | Not | Not | Not | Not | Not |
| | | | | detected | detected | detected | detected | detected |
| Q9R098 | Hepatocyte | HGFAC | 8.74E+05 | 1.03E+06 | 6.60E+05 | 5.67E+06 | 9.34E+06 | 7.83E+06 |
| | growth | | | | | | | |
| | factor | | | | | | | |
| | activator | | | | | | | |
| Q61646 | Haptoglobin | HP | 1.02E+09 | 1.13E+09 | 9.61E+08 | 4.33E+09 | 3.45E+09 | 5.26E+09 |
| P98086 | Complement | C1QA | 5.50E+06 | 4.95E+06 | 5.74E+06 | 8.33E+07 | 5.19E+07 | 4.37E+07 |
| | C1q | | | | | | | |
| | subcomponent | | | | | | | |
| | subunit A | | | | | | | |
| Q61247 | Alpha- | SERPIN | 4.97E+07 | 3.09E+07 | 2.60E+07 | 1.59E+08 | 1.60E+08 | 1.49E+08 |
| | 2- | F2 | | | | | | |
| | antiplasmin | | | | | | | |
| Q9Z1R3 | Apolipoprotein M | APOM | 1.66E+06 | 1.87E+06 | 3.15E+06 | 2.20E+07 | 1.55E+07 | 1.23E+07 |
| P26041 | Moesin | MSN | Not | Not | Not | 3.24E+05 | 5.27E+04 | 3.85E+05 |
| | | | detected | detected | detected | | | |
| P35441 | Thrombospondin-1 | THBS1 | 1.06E+07 | 1.26E+07 | 1.15E+07 | 1.15E+08 | 2.05E+08 | 3.40E+08 |
| Q02105 | Complement | C1QC | 5.77E+06 | 4.10E+06 | 9.06E+06 | 8.11E+07 | 6.05E+07 | 4.38E+07 |
| | C1q | | | | | | | |
| | subcomponent | | | | | | | |
| | subunit C | | | | | | | |
| O88783 | Coagulation | F5 | 5.66E+05 | 5.09E+05 | 4.01E+05 | 1.03E+07 | 4.73E+06 | 1.60E+07 |
| | factor V | | | | | | | |
| P14106 | Complement | C1QB | 1.66E+07 | 1.84E+07 | 1.23E+07 | 2.18E+08 | 9.06E+07 | 1.14E+08 |
| | C1q | | | | | | | |
| | subcomponent | | | | | | | |
| | subunit B | | | | | | | |
| P01029 | Complement | C4B | 4.70E+07 | 7.13E+07 | 2.93E+07 | 5.97E+08 | 4.88E+08 | 9.63E+08 |
| | C4-B | | | | | | | |
| P01872 | Immunoglobulin | IGHM | 2.14E+08 | 2.50E+08 | 1.55E+08 | 2.40E+09 | 1.49E+09 | 3.65E+09 |
| | heavy | | | | | | | |
| | constant | | | | | | | |
| | mu | | | | | | | |
| P01878 | Ig alpha | | 3.87E+08 | 3.01E+08 | 2.11E+08 | 1.25E+09 | 1.74E+09 | 1.75E+09 |
| | chain C | | | | | | | |
| | region | | | | | | | |
| P21180 | Complement C2 | C2 | 7.54E+05 | 8.99E+05 | 2.92E+05 | 1.13E+07 | 1.01E+07 | 1.44E+07 |
| Q61233 | Plastin-2 | LCP1 | 5.26E+05 | 3.28E+05 | 1.96E+05 | 6.14E+05 | 5.73E+05 | 4.66E+05 |
| P01898 | H-2 Class I | H2-Q10 | 3.56E+07 | 5.22E+07 | 1.79E+07 | 4.02E+08 | 2.53E+08 | 4.63E+08 |
| | histocompatibility | | | | | | | |
| | antigen, Q10 | | | | | | | |
| | alpha chain | | | | | | | |
| P14847 | C-reactive | CRP | 6.92E+05 | 5.00E+05 | 1.10E+06 | 2.15E+07 | 1.10E+07 | 9.75E+06 |
| | protein | | | | | | | |
| P14426 | H-2 Class I | H2-D1 | 7.21E+05 | 9.16E+05 | 3.23E+05 | 8.76E+06 | 6.23E+06 | 1.08E+07 |
| | histocompatibility | | | | | | | |
| | antigen, D-K | | | | | | | |
| | alpha chain | | | | | | | |
| Q61696 | Heat shock | HSPA1A | 3.00E+06 | 3.75E+06 | 5.92E+06 | 4.72E+06 | 2.19E+06 | 5.72E+06 |
| | 70 kDa | | | | | | | |
| | protein 1A | | | | | | | |
| P08226 | Apolipoprotein | APOE | 1.85E+07 | 2.11E+07 | 1.48E+07 | 5.22E+07 | 4.19E+07 | 6.42E+07 |
| | E | | | | | | | |
| P01791 | Ig heavy chain | | 1.42E+06 | 1.40E+06 | 2.44E+06 | 1.87E+07 | 1.74E+07 | 3.02E+07 |
| | V region | | | | | | | |
| | HPCM 6 | | | | | | | |
| P12246 | Serum amyloid | APCS | 7.48E+07 | 5.26E+07 | 9.80E+07 | 8.15E+08 | 4.68E+08 | 3.85E+08 |
| | P-component | | | | | | | |
| P01631 | Ig kappa chain | | 1.65E+08 | 1.25E+08 | 2.37E+08 | 1.20E+09 | 9.16E+08 | 6.80E+08 |
| | V-II region | | | | | | | |
| | 26-10 | | | | | | | |
| P62806 | Histone H4 | H4F16 | 2.26E+07 | 3.32E+07 | 1.08E+08 | 1.08E+07 | 5.92E+06 | 2.74E+06 |
| P01632 | Ig kappa chain | IGKV7-33 | 3.67E+07 | 3.01E+07 | 5.79E+07 | 2.96E+08 | 2.04E+08 | 1.68E+08 |
| | V-I region | | | | | | | |
| | S107A | | | | | | | |
| P01844 | Ig lambda-2 | IGLC2 | 4.49E+06 | 2.15E+06 | 6.61E+06 | 6.58E+07 | 4.69E+07 | 4.02E+07 |
| | chain C region | | | | | | | |
| P01843 | Ig lambda-1 | | 1.62E+07 | 1.10E+07 | 2.53E+07 | 2.14E+08 | 1.53E+08 | 9.50E+07 |
| | chain C region | | | | | | | |
| O70362 | Phosphatidylinositol- | GPLD1 | 1.14E+07 | 9.88E+06 | 7.27E+06 | 4.88E+07 | 1.22E+08 | 1.24E+08 |
| | glycan-specific | | | | | | | |
| | phospholipase D | | | | | | | |
| A6X935 | Inter alpha- | ITIH4 | 3.11E+08 | 2.13E+08 | 1.33E+08 | 7.99E+08 | 1.30E+09 | 9.49E+08 |
| | trypsin inhibitor, | | | | | | | |
| | heavy chain 4 | | | | | | | |
| P28666 | Murinoglobulin-2 | MUG2 | 1.89E+07 | 2.25E+07 | 1.39E+07 | 1.89E+08 | 5.36E+08 | 1.01E+09 |
| P01630 | Ig kappa chain | | 1.31E+07 | 9.57E+06 | 2.26E+07 | 1.55E+08 | 1.18E+08 | 7.31E+07 |
| | V-II region | | | | | | | |
| | 7S34.1 | | | | | | | |
| P01638 | Ig kappa chain | | 1.61E+07 | 1.33E+07 | 3.18E+07 | 2.40E+08 | 1.40E+08 | 1.09E+08 |
| | V-V region | | | | | | | |
| | L6 (Fragment) | | | | | | | |
| Q923 D2 | Flavin | BLVRB | 6.19E+05 | 2.47E+05 | 2.79E+0.5 | 3.82E+06 | 2.80 | 2.15E+06 |
| | reductase | | | | | | | |
| | (NADPH) | | | | | | | |
| P01635 | Immunoglobulin | IGKV12-41 | 8.63E+07 | 7.01E+07 | 1.46E+08 | 7.71E+08 | 5.26E+08 | 4.20E+08 |
| | kappa chain | | | | | | | |
| | variable 12-41 | | | | | | | |
| | (Fragment) | | | | | | | |
| P01728 | Ig lambda-2 | | 3.43E+06 | 3.42E+06 | 7.00E+06 | 3.59E+07 | 2.29E+07 | 1.83E+07 |
| | chain V region | | | | | | | |
| P84750 | Ig kappa chain | | 7.60E+06 | 5.04E+06 | 1.32E+07 | 8.51E+07 | 6.07E+07 | 4.46E+07 |
| | V region | | | | | | | |
| | Mem5 (Fragment) | | | | | | | |
| Q60994 | Adiponectin | ADIPOQ | 1.43E+07 | 2.63E+07 | 8.52E+06 | 1.34E+08 | 7.88E+07 | 1.32E+08 |
| Q07968 | Coagulation | F13B | 8.44E+05 | 1.28E+06 | 2.97E+05 | 1.79E+07 | 9.09E+06 | 2.23E+07 |
| | factor XIII | | | | | | | |
| | B chain | | | | | | | |
| Q60590 | Alpha-1-acid | ORM1 | 1.66E+07 | 1.48E+07 | 2.92E+07 | 1.62E+08 | 1.61E+08 | 2.89E+08 |
| | glycoprotein 1 | | | | | | | |
| P09103 | Protein | P4HB | 4.73E+05 | 1.61E+06 | 1.93E+06 | 3.07E+05 | 4.67E+05 | 7.23E+05 |
| | disulfide- | | | | | | | |
| | isomerase | | | | | | | |
| P08607 | C4b-binding | C4BPA | 1.67E+07 | 2.38E+07 | 1.01E+07 | 1.42E+08 | 8.89E+07 | 2.30E+08 |
| | protein | | | | | | | |
| Q61702 | Inter-alpha- | ITIH1 | 1.13E+07 | 1.59E+07 | 4.90E+06 | 1.21E+08 | 2.11E+08 | 1.91E+08 |
| | trypsin inhibitor | | | | | | | |
| | heavy chain H1 | | | | | | | |
| Q07456 | Protein AMBP | AMBP | 1.78E+07 | 1.47E+07 | 8.54E+06 | 8.29E+07 | 1.98E+08 | 2.28E+08 |
| Q9JJN5 | Carboxypeptidase | CPN1 | 2.22E+06 | 3.00E+06 | 7.65E+06 | 5.05E+07 | 4.69E+07 | 6.89E+07 |
| | N catalytic chain | | | | | | | |
| Q80YC5 | Coagulation | F12 | 4.67E+06 | 2.43E+06 | 1.14E+06 | 2.94E+07 | 2.88E+07 | 4.49E+07 |
| | factor XII | | | | | | | |
| P06909 | Complement | CFH | 6.63E+07 | 9.01E+07 | 4.68E+07 | 5.13E+08 | 1.24E+09 | 2.31E+09 |
| | factor H | | | | | | | |
| P01837 | Immunoglobulin | IGKC | 6.82E+08 | 7.05E+08 | 1.44E+09 | 4.76E+09 | 3.84E+09 | 3.28E+09 |
| | kappa constant | | | | | | | |
| P01636 | Ig kappa chain | | 3.97E+07 | 2.83E+07 | 6.64E+07 | 4.10E+08 | 2.67E+08 | 1.96E+08 |
| | V-V region | | | | | | | |
| | MOPC 149 | | | | | | | |
| O08742 | Platelet | GP5 | 2.35E+05 | 6.35E+05 | 1.77E+05 | 5.36E+06 | 3.19E+06 | 7.86E+06 |
| | glycoprotein V | | | | | | | |
| P48678 | Prelamin-A/C | LMNA | 3.67E+07 | 9.70E+07 | 1.01E+07 | 2.28E+05 | 6.54E+05 | 8.63E+05 |
| Q9DBD0 | Inhibitor of | ICA | 2.62E+07 | 4.07E+07 | 1.09E+07 | 5.07E+08 | 2.68E+08 | 7.64E+08 |
| | carbonic | | | | | | | |
| | anhydrase | | | | | | | |
| Q921I1 | Serotransferrin | TF | 8.14E+09 | 1.13E+10 | 4.70E+09 | 9.07E+10 | 5.01E+10 | 1.25E+11 |
| P29788 | Vitronectin | VTN | 2.36E+07 | 1.32E+07 | 9.25E+06 | 9.30E+07 | 1.29E+08 | 9.23E+07 |
| Q61704 | Inter-alpha- | ITIH3 | 2.63E+07 | 2.82E+07 | 1.56E+07 | 1.33E+08 | 3.77E+08 | 5.38E+08 |
| | trypsin inhibitor | | | | | | | |
| | heavy chain H3 | | | | | | | |
| P01642 | Ig kappa chain | GM10881 | 6.05E+06 | 3.57E+06 | 9.95E+06 | 7.44E+07 | 4.68E+07 | 3.87E+07 |
| | V-V region | | | | | | | |
| | L7 (Fragment) | | | | | | | |
| P28665 | Murinoglobulin-1 | MUG1 | 3.57E+08 | 3.87E+08 | 2.69E+08 | 1.99E+09 | 5.40E+09 | 1.00E+10 |
| P97350 | Plakophilin-1 | PKP1 | 2.25E+06 | 2.63E+06 | Not | Not | Not | Not |
| | | | | | detected | detected | detected | detected |
| Q8VCG4 | Complement | C8G | 3.41E+06 | 2.26E+06 | 6.16E+06 | 3.83E+07 | 2.30E+07 | 1.74E+07 |
| | component C8 | | | | | | | |
| | gamma chain | | | | | | | |
| Q8CG16 | Complement | C1RA | 4.02E+06 | 3.72E+06 | 1.61E+06 | 1.32E+07 | 2.69E+07 | 2.01E+07 |
| | C1r-A | | | | | | | |
| | subcomponent | | | | | | | |
| Q61703 | Inter-alpha- | ITIH2 | 2.50E+07 | 2.44E+07 | 1.46E+07 | 1.25E+08 | 3.87E+08 | 2.98E+08 |
| | trypsin inhibitor | | | | | | | |
| | heavy chain H2 | | | | | | | |
| Q61147 | Ceruloplasmin | CP | 1.60E+08 | 1.99E+08 | 1.16E+08 | 9.74E+08 | 2.80E+09 | 5.03E+09 |
| Q61838 | Pregnancy | PZP | 4.07E+09 | 5.65E+09 | 2.56E+09 | 1.66E+10 | 2.62E+10 | 5.23E+10 |
| | zone protein | | | | | | | |
| P01807 | Ig heavy chain | | 5.15E+07 | 5.04E+07 | 1.11E+08 | 3.72E+08 | 4.88E+08 | 8.60E+08 |
| | V region X44 | | | | | | | |
| P00920 | Carbonic | CA2 | 3.20E+06 | 1.92E+06 | 3.89E+06 | 4.37E+07 | 2.44E+07 | 1.74E+07 |
| | anhydrase 2 | | | | | | | |
| P01746 | Ig heavy chain | | 9.21E+06 | 1.31E+07 | 2.16E+07 | 9.31E+07 | 1.22E+08 | 1.99E+08 |
| | V region 93G7 | | | | | | | |
| Q8BND5 | Sulfhydryl | QSOX1 | 1.58E+07 | 1.11E+07 | 5.35E+06 | 6.89E+07 | 7.06E+07 | 6.06E+07 |
| | oxidase 1 | | | | | | | |
| P23953 | Carboxylesterase 1C | CES1C | 4.60E+08 | 4.57E+08 | 2.13E+08 | 3.25E+09 | 1.85E+09 | 5.03E+09 |
| P01723 | Ig lambda-1 | | 8.50E+06 | 6.32E+06 | 1.57E+07 | 8.56E+07 | 5.14E+07 | 4.50E+07 |
| | chain V region | | | | | | | |
| Q62351 | Transferrin | TFRC | 1.38E+06 | 4.87E+05 | 8.72E+05 | 7.60E+06 | 2.68E+07 | 6.94E+06 |
| | receptor | | | | | | | |
| | protein 1 | | | | | | | |
| Q91Y97 | Fructose- | ALDOB | 2.39E+05 | 3.77E+05 | 1.14E+05 | 1.90E+06 | 1.13E+06 | 2.20E+06 |
| | bisphosphate | | | | | | | |
| | aldolase B | | | | | | | |
| P20918 | Plasminogen | PLG | 1.79E+08 | 7.59E+07 | 1.23E+08 | 7.21E+08 | 2.05E+09 | 5.68E+08 |
| O70456 | 14-3-3 | SFN | 2.10E+07 | 6.09E+07 | 1.03E+08 | 1.84E+07 | 1.19E+07 | 1.27E+07 |
| | protein | | | | | | | |
| | sigma | | | | | | | |
| Q60930 | Voltage- | VDAC2 | Not | 8.89E+05 | 3.02E+05 | Not | Not | Not |
| | dependent | | detected | | | detected | detected | detected |
| | anion- | | | | | | | |
| | selective | | | | | | | |
| | channel | | | | | | | |
| | protein 2 | | | | | | | |
| P01801 | Ig heavy chain | | 2.33E+07 | 2.21E+07 | 4.38E+07 | 1.72E+08 | 1.96E+08 | 3.12E+08 |
| | V-III region J606 | | | | | | | |
| P68372 | Tubulin beta- | TUBB4B | 3.95E+06 | 9.94E+06 | 2.11E+07 | 2.90E+06 | 2.90E+06 | 4.57E+06 |
| | 4B chain | | | | | | | |
| P01633 | Immunoglobulin | IGKV6-17 | 1.26E+08 | 8.51E+07 | 2.14E+08 | 1.26E+09 | 6.54E+08 | 4.96E+08 |
| | kappa chain | | | | | | | |
| | variable 6-17 | | | | | | | |
| O88947 | Coagulation | F10 | 4.43E+06 | 4.94E+06 | 8.05E+06 | 3.92E+07 | 4.43E+07 | 9.25E+07 |
| | factor X | | | | | | | |
| P32261 | Antithrombin-III | SERPIN | 9.22E+07 | 7.55E+07 | 5.82E+07 | 2.39E+08 | 3.42E+08 | 1.64E+08 |
| | | C1 | | | | | | |
| P01660 | Ig kappa chain | | 1.24E+07 | 8.10E+06 | 2.15E+07 | 1.11E+08 | 7.34E+07 | 5.79E+07 |
| | V-III region | | | | | | | |
| | PC3741/TEPC111 | | | | | | | |
| Q9ESB3 | Histidine-rich | HRG | 2.08E+08 | 1.88E+08 | 1.06E+08 | 4.68E+08 | 3.20E+08 | 5.55E+08 |
| | glycoprotein | | | | | | | |
| P46412 | Glutathione | GPX3 | 2.62E+06 | 3.51E+06 | 8.41E+06 | 2.45E+07 | 2.37E+07 | 1.42E+07 |
| | peroxidase 3 | | | | | | | |
| P26262 | Plasma | KLKB1 | 5.26E+06 | 3.13E+06 | 9.94E+06 | 3.55E+07 | 5.52E+07 | 8.25E+07 |
| | kallikrein | | | | | | | |
| Q8R121 | Protein Z- | SERPIN | 5.24E+06 | 1.42E+06 | 1.05E+06 | 1.39E+07 | 1.74E+07 | 2.32E+07 |
| | dependent | A10 | | | | | | |
| | protease | | | | | | | |
| | inhibitor | | | | | | | |
| P04186 | Complement | CFB | 7.29E+07 | 3.13E+07 | 3.68E+07 | 2.22E+08 | 5.10E+08 | 1.55E+08 |
| | factor B | | | | | | | |
| P01027 | Complement C3 | C3 | 4.41E+09 | 3.59E+09 | 1.99E+09 | 1.61E+10 | 1.30E+10 | 1.19E+10 |
| Q03734 | Serine protease | SERPIN | 1.04E+07 | 6.79E+06 | 5.91E+06 | 2.78E+07 | 4.57E+07 | 2.47E+07 |
| | inhibit or A3M | A3M | | | | | | |
| P01867 | Immunoglobulin | IGHG2B | 6.98E+08 | 7.66E+08 | 1.92E+09 | 6.98E+09 | 8.99E+09 | 1.46E+10 |
| | heavy constant | | | | | | | |
| | gamma 2B | | | | | | | |
| P20152 | Vimentin | VIM | 6.79E+07 | 2.14E+08 | 1.50E+08 | 2.74E+07 | 1.60E+07 | 2.32E+07 |
| P01869 | Ig gamma-1 | IGHG1 | 5.97E+08 | 5.28E+08 | 1.33E+09 | 4.79E+09 | 5.89E+09 | 9.48E+09 |
| | chain C | | | | | | | |
| | region, | | | | | | | |
| | membrane- | | | | | | | |
| | bound form | | | | | | | |
| P18527 | Ig heavy chain | | 4.98E+06 | 3.35E+06 | 9.83E+06 | 4.50E+07 | 4.06E+07 | 7.71E+07 |
| | V region 914 | | | | | | | |
| P01865 | Ig gamma-2A | IGH-1A | 1.13E+09 | 1.18E+09 | 2.69E+09 | 9.19E+09 | 1.11E+10 | 1.77E+10 |
| | chain C region, | | | | | | | |
| | membrane- | | | | | | | |
| | bound form | | | | | | | |
| P01864 | Ig gamma-2A | | 4.72E+08 | 5.26E+08 | 1.22E+09 | 4.33E+09 | 5.17E+09 | 7.16E+09 |
| | chain C region | | | | | | | |
| | secreted form | | | | | | | |
| P18531 | Ig heavy chain | IGHV3-6 | 3.28E+06 | 2.85E+06 | 8.60E+06 | 3.98E+07 | 4.11E+07 | 8.46E+07 |
| | V region 3-6 | | | | | | | |
| P49182 | Heparin | SERPIN | 1.59E+07 | 9.24E+06 | 3.56E+06 | 1.03E+08 | 6.64E+07 | 9.42E+07 |
| | cofactor 2 | D1 | | | | | | |
| Q6GQT1 | Alpha-2- | A2M | 2.04E+05 | 3.20E+05 | 4.96E+04 | 1.91E+06 | 5.48E+06 | 9.95E+06 |
| | macroglobulin-P | | | | | | | |
| Q64726 | Zinc-alpha- | AZGP1 | 1.11E+07 | 6.86E+06 | 2.02E+07 | 8.94E+07 | 9.67E+07 | 1.62E+08 |
| | 2-glycoprotein | | | | | | | |
| Q8VCS0 | N-acetyl | PGLYRP2 | 1.52E+06 | 1.93E+06 | 4.77E+05 | 1.79E+07 | 9.50E+06 | 2.24E+07 |
| | muramoyl-L- | | | | | | | |
| | alanine | | | | | | | |
| | amidase | | | | | | | |
| P06683 | Complement | C9 | 3.31E+05 | 5.62E+05 | 2.47E+05 | 5.03E+06 | 5.24E+06 | 7.82E+06 |
| | component C9 | | | | | | | |
| P70274 | Selenoprotein P | SELENOP | 3.02E+05 | 2.24E+05 | 4.81E+05 | 2.65E+06 | 3.98E+06 | 9.22E+06 |
| Q06890 | Clusterin | CLU | 3.48E+07 | 4.15E+07 | 1.99E+07 | 8.39E+07 | 7.20E+07 | 1.41E+08 |
| P17182 | Alpha- | ENO1 | 1.78E+06 | 2.21E+05 | 4.80E+06 | 2.49E+05 | Not | Not |
| | enolase | | | | | | detected | detected |
| P10126 | Elongation | EEF1A1 | 2.36E+07 | 5.98E+07 | 1.60E+08 | 1.48E+07 | 7.21E+06 | 1.01E+07 |
| | factor | | | | | | | |
| | 1-alpha 1 | | | | | | | |
| Q8CG14 | Complement | C1S1 | 3.91E+06 | 1.02E+06 | 1.78E+06 | 8.86E+06 | 3.30E+07 | 1.56E+07 |
| | C1s-1 | | | | | | | |
| | subcomponent | | | | | | | |
| P07361 | Alpha-1-acid | ORM2 | 1.39E+06 | 1.32E+06 | 7.32E+06 | 2.39E+07 | 2.68E+07 | 3.00E+07 |
| | glycoprotein 2 | | | | | | | |
| P13020 | Gelsolin | GSN | 1.03E+08 | 4.51E+07 | 8.13E+07 | 3.08E+08 | 9.56E+08 | 3.03E+08 |
| O08677 | Kininogen-1 | KNG1 | 3.06E+08 | 1.87E+08 | 1.32E+08 | 5.33E+08 | 7.16E+08 | 5.38E+08 |
| Q61129 | Complement | CFI | 9.47E+06 | 9.91E+06 | 1.64E+07 | 4.94E+07 | 1.13E+08 | 7.01E+07 |
| | factor I | | | | | | | |
| O88968 | Transcobalamin-2 | TCN2 | Not | Not | Not | 6.15E+05 | 8.90E+05 | 9.99E+05 |
| | | | detected | detected | detected | | | |
| P02089 | Hemoglobin | HBB-B2 | 1.86E+06 | 2.78E+06 | 4.16E+06 | 1.22E+07 | 1.07E+07 | 6.70E+06 |
| | subunit | | | | | | | |
| | beta-2 | | | | | | | |
| P97290 | Plasma | SERPIN | 7.84E+07 | 2.34E+07 | 3.17E+07 | 1.51E+08 | 4.19E+08 | 1.92E+08 |
| | protease | G1 | | | | | | |
| | C1 inhibitor | | | | | | | |
| P01670 | Ig kappa chain | | 1.24E+07 | 3.56E+06 | 1.14E+07 | 9.20E+07 | 5.35E+07 | 4.97E+07 |
| | V-III region | | | | | | | |
| | PC 6684 | | | | | | | |
| Q02257 | Junction | JUP | 6.50E+06 | 1.01E+07 | 1.10E+05 | 1.28E+05 | 4.02E+04 | 1.30E+05 |
| | plakoglobin | | | | | | | |
| Q01339 | Beta-2- | APOH | 7.95E+07 | 9.25E+07 | 1.53E+08 | 3.66E+08 | 6.06E+08 | 4.07E+08 |
| | glycoprotein 1 | | | | | | | |
| P11859 | Angiotensinogen | AGT | 3.35E+06 | 2.71E+06 | 8.28E+06 | 3.24E+07 | 4.16E+07 | 4.19E+07 |
| P21614 | Vitamin D- | GC | 1.50E+08 | 1.39E+08 | 3.96E+08 | 1.28E+09 | 1.85E+09 | 2.69E+09 |
| | binding protein | | | | | | | |
| P03987 | Ig gamma-3 | | 1.37E+08 | 1.21E+08 | 3.66E+08 | 1.15E+09 | 1.46E+09 | 2.15E+09 |
| | chain C region | | | | | | | |
| P97298 | Pigment | SERPIN | 3.38E+06 | 2.45E+06 | 6.49E+06 | 2.99E+07 | 3.71E+07 | 6.04E+07 |
| | epithelium- | F1 | | | | | | |
| | derived | | | | | | | |
| | factor | | | | | | | |
| P18524 | Ig heavy chain | | 3.19E+07 | 2.88E+07 | 6.94E+07 | 1.98E+08 | 2.66E+08 | 3.86E+08 |
| | V region RF | | | | | | | |
| Q8VCM7 | Fibrinogen | FGG | Not | Not | 3.25E+06 | 4.67E+05 | Not | Not |
| | gamma chain | | detected | detected | | | detected | detected |
| P11276 | Fibroectin | nFN1 | 4.63E+07 | 5.56E+07 | 1.40E+07 | 2.38E+08 | 1.04E+09 | 1.02E+09 |
| P07356 | Annexin A2 | ANXA2 | 1.94E+07 | 4.34E+07 | 1.37E+08 | 2.11E+07 | 1.72E+07 | 1.36E+07 |
| Q9Z126 | Platelet | PF4 | 1.86E+07 | 2.58E+07 | 3.36E+07 | 1.41E+08 | 1.37E+08 | 5.30E+07 |
| | factor 4 | | | | | | | |
| Q00724 | Retinol | RBP4 | 6.20E+06 | 8.30E+06 | 2.34E+07 | 6.25E+07 | 4.03E+07 | 3.70E+07 |
| | binding | | | | | | | |
| | protein 4 | | | | | | | |
| Q8BTM8 | Filamin-A | FLNA | Not | 5.66E+04 | 3.06E+04 | 3.11E+05 | 7.09E+05 | 1.66E+06 |
| | | | detected | | | | | |
| P42703 | Leukemia | LIFR | 1.65E+06 | 1.12E+06 | 6.78E+05 | 3.39E+06 | 8.66E+06 | 1.41E+07 |
| | inhibitory | | | | | | | |
| | factor | | | | | | | |
| | receptor | | | | | | | |
| P11247 | Myeloperoxidase | MPO | 8.11E+05 | 3.64E+05 | 1.69E+07 | 2.65E+06 | Not | Not |
| | | | | | | | detected | detected |
| P11680 | Properdin | CFP | 2.41E+06 | 2.05E+06 | 4.34E+06 | 1.73E+07 | 3.26E+07 | 2.74E+07 |
| P06330 | Ig heavy chain V | | 2.29E+08 | 2.74E+08 | 6.13E+08 | 1.21E+09 | 2.28E+09 | 3.54E+09 |
| | Region AC38 205.12 | | | | | | | |
| P70389 | Insulin-like | IGFALS | 6.86E+06 | 1.20E+06 | 2.59E+06 | 1.38E+07 | 5.29E+07 | 1.92E+07 |
| | growth factor- | | | | | | | |
| | binding protein | | | | | | | |
| | complex acid | | | | | | | |
| | labile subunit | | | | | | | |
| P06336 | Ig epsilon | | 3.35E+06 | 5.36E+05 | 1.24E+06 | 8.24E+06 | 2.82E+07 | 8.26E+06 |
| | chain C region | | | | | | | |
| Q91X72 | Hemopexin | HPX | 5.83E+09 | 4.44E+09 | 2.22E+09 | 1.52E+10 | 1.03E+10 | 2.17E+10 |
| Q03311 | Cholinesterase | BCHE | 2.84E+05 | Not | Not | 6.73E+05 | 2.42E+06 | 7.68E+05 |
| | | | | detected | detected | | | |
| Q9QWK4 | CD5 antigen- | CD5L | 1.96E+07 | 1.76E+07 | 4.54E+07 | 1.42E+08 | 2.07E+08 | 1.27E+08 |
| | like | | | | | | | |
| P07309 | Transthyretin | TTR | 6.17E+08 | 6.98E+08 | 8.07E+08 | 1.68E+09 | 1.46E+09 | 1.11E+09 |
| Q06770 | Corticosteroid- | SERPIN | 2.13E+07 | 1.19E+07 | 6.55E+06 | 6.35E+07 | 8.05E+07 | 4.41E+07 |
| | binding globulin | A6 | | | | | | |
| P26039 | Talin-1 | TLN1 | 5.44E+04 | Not | Not | 2.32E+05 | 2.32E+06 | 3.62E+06 |
| | | | | detected | detected | | | |
| Q9D2Q8 | Protein | S100A14 | 4.64E+05 | Not | 1.60E+07 | 9.17E+05 | Not | 1.54E+05 |
| | S100-A14 | | | detected | | | detected | |
| O89020 | Afamin | AFM | 9.84E+07 | 2.24E+07 | 3.15E+07 | 1.23E+08 | 2.30E+08 | 1.57E+08 |
| Q9DBB9 | Carboxypeptidase | CPN2 | 1.41E+07 | 2.31E+06 | 5.55E+06 | 2.23E+07 | 5.21E+07 | 2.28E+07 |
| | N subunit 2 | | | | | | | |
| P10107 | Annexin A1 | ANXA1 | 3.37E+07 | 2.20E+07 | 9.56E+08 | 6.51E+07 | 1.02E+07 | 2.65E+06 |
| P01749 | Ig heavy chain | IGHV1-61 | 1.88E+06 | 2.21E+06 | 7.53E+06 | 1.84E+07 | 3.56E+07 | 5.13E+07 |
| | V region 3 | | | | | | | |
| E9Q414 | Apolipoprotein | APOB | 1.03E+07 | 2.23E+07 | 1.66E+07 | 3.48E+07 | 1.07E+08 | 2.01E+08 |
| | B-100 | | | | | | | |
| P01887 | Beta-2- | B2M | 6.30E+06 | 1.35E+07 | 1.46E+07 | 4.89E+07 | 4.95E+07 | 2.04E+07 |
| | microglobulin | | | | | | | |
| P09581 | Macrophage | CSF1R | 1.75E+05 | Not | Not | 8.09E+05 | 4.87E+06 | 6.92E+05 |
| | colony | | | detected | detected | | | |
| | stimulating | | | | | | | |
| | factor 1 | | | | | | | |
| | receptor | | | | | | | |
| P63017 | Heat shock | HSPA8 | 3.59E+06 | 2.97E+07 | 1.43E+07 | 2.47E+06 | 3.48E+06 | 4.26E+06 |
| | cognate | | | | | | | |
| | 71 kDa | | | | | | | |
| | protein | | | | | | | |
| Q60963 | Platelet- | PLA2G7 | 1.34E+06 | 2.52E+05 | 4.30E+05 | 6.45E+06 | 5.38E+06 | 3.07E+06 |
| | activating | | | | | | | |
| | factor | | | | | | | |
| | acetylhydrolase | | | | | | | |
| Q9JHH6 | Carboxypeptidase | CPB2 | 1.61E+06 | 2.14E+06 | 7.63E+05 | 1.47E+07 | 1.17E+07 | 1.38E+07 |
| | B2 | | | | | | | |
| Q08879 | Fibulin-1 | FBLN1 | 1.86E+05 | Not | 9.35E+04 | 2.88E+05 | 1.81E+06 | 4.16E+05 |
| | | | | detected | | | | |
| P21107 | Tropomyosin | TPM3 | 2.70E+07 | 1.41E+08 | 4.34E+08 | 1.09E+07 | 9.20E+06 | 1.37E+07 |
| | alpha-3 chain | | | | | | | |
| P07901 | Heat shock | HSP90A | Not | 6.27E+04 | 4.41E+06 | Not | Not | Not |
| | protein HSP | A1 | detected | | | detected | detected | detected |
| | 90-alpha | | | | | | | |
| P82198 | Transforming | TGFBI | 1.69E+06 | 1.02E+06 | 3.30E+05 | 4.75E+06 | 4.86E+06 | 2.71E+06 |
| | growth factor- | | | | | | | |
| | beta-induced | | | | | | | |
| | protein ig-h3 | | | | | | | |
| P07724 | Albumin | ALB | 1.41E+11 | 1.08E+11 | 5.09E+10 | 3.57E+11 | 3.90E+11 | 2.29E+11 |
| P20029 | Endoplasmic | HSPA5 | 8.63E+06 | 5.04E+07 | 2.23E+07 | 3.59E+06 | 3.23E+06 | 5.52E+06 |
| | reticulum | | | | | | | |
| | chaperone BiP | | | | | | | |
| P11499 | Heat shock | HSP90A | 6.78E+06 | 2.02E+07 | 1.72E+07 | 2.09E+06 | 2.53E+06 | 5.02E+06 |
| | protein HSP | B1 | | | | | | |
| | 90-beta | | | | | | | |
| P16301 | Phosphatidylcholine- | LCAT | 9.58E+05 | 9.08E+05 | 1.65E+05 | 3.05E+06 | 1.54E+06 | 1.50E+06 |
| | sterolacyltransferase | | | | | | | |
| P31532 | Serum | SAA4 | 1.50E+07 | 2.04E+07 | 3.90E+07 | 8.50E+07 | 3.42E+07 | 3.77E+07 |
| | amyloid A-4 | | | | | | | |
| | protein | | | | | | | |
| Q01279 | Epidermal | EGFR | 1.15E+07 | 2.53E+06 | 5.55E+06 | 1.70E+07 | 5.73E+07 | 1.70E+07 |
| | growth factor | | | | | | | |
| | receptor | | | | | | | |
| P10605 | Cathepsin B | CTSB | Not | 7.93E+05 | Not | 3.56E+06 | 8.72E+05 | 5.75E+06 |
| | | | detected | | detected | | | |
| P17742 | Peptidyl- | PPIA | Not | Not | 6.30E+07 | 4.52E+06 | Not | Not |
| | prolyl | | detected | detected | | | detected | detected |
| | cis-trans | | | | | | | |
| | isomerase A | | | | | | | |
| P29699 | Alpha-2-HS- | AHSG | 1.29E+08 | 1.50E+08 | 1.68E+08 | 2.76E+08 | 5.02E+08 | 4.75E+08 |
| | glycoprotein | | | | | | | |

| | Normalized | | | P-value | Abundance ratio | | |
|---|---|---|---|---|---|---|---|
| Accession | abundance_L-Cb | | | (one-way | D-Cb/ | D-Cb/ | L-Cb/ |
| No. | 1 | 2 | 3 | ANOVA) | L-Cb^{a} | Bead^{a} | Bead^{a} |
| P19221 | 2.80E+07 | 2.81E+07 | 2.76E+07 | 6.59E−07 | 5.29 | 6.23 | 1.18 |
| E9Q557 | 1.18E+08 | 1.30E+08 | 1.95E+08 | 3.11E−06 | 0.00 | 0.02 | 17.37 |
| P06684 | 1.81E+07 | 1.50E+07 | 1.18E+07 | 9.60E−06 | 11.65 | 17.97 | 1.54 |
| P52430 | 2.57E+07 | 1.80E+07 | 2.24E+07 | 1.59E−05 | 7.33 | 10.93 | 1.49 |
| Q9R269 | 8.20E+06 | 1.59E+07 | 2.12E+07 | 2.71E−05 | — | — | 82.95 |
| Q9R098 | 7.40E+05 | 1.00E+06 | 1.01E+06 | 2.87E−05 | 8.31 | 8.91 | 1.07 |
| Q61646 | 1.24E+09 | 1.03E+09 | 1.30E+09 | 4.15E−05 | 3.66 | 4.20 | 1.15 |
| P98086 | 5.51E+06 | 9.13E+06 | 5.25E+06 | 5.92E−05 | 8.99 | 11.05 | 1.23 |
| Q61247 | 3.99E+07 | 3.31E+07 | 3.46E+07 | 1.42E−04 | 4.35 | 4.39 | 1.01 |
| Q9Z1R3 | 2.67E+06 | 2.44E+06 | 2.69E+06 | 1.45E−04 | 6.38 | 7.44 | 1.17 |
| P26041 | 1.23E+07 | 1.58E+07 | 2.14E+07 | 1.97E−04 | 0.02 | — | — |
| P35441 | 2.59E+07 | 2.87E+07 | 1.51E+07 | 2.11E−04 | 9.45 | 18.95 | 2.00 |
| Q02105 | 7.88E+06 | 1.11E+07 | 7.07E+06 | 2.46E−04 | 7.11 | 9.79 | 1.38 |
| O88783 | 1.32E+06 | 8.04E+05 | 8.70E+05 | 2.58E−04 | 10.34 | 20.96 | 2.03 |
| P14106 | 1.84E+07 | 2.33E+07 | 1.64E+07 | 2.61E−04 | 7.28 | 8.94 | 1.23 |
| P01029 | 1.26E+08 | 8.03E+07 | 7.66E+07 | 2.73E−04 | 7.25 | 13.88 | 1.91 |
| P01872 | 4.06E+08 | 2.26E+08 | 3.83E+08 | 3.03E−04 | 7.42 | 12.17 | 1.64 |
| P01878 | 3.93E+08 | 2.77E+08 | 2.28E+08 | 3.19E−04 | 5.29 | 5.28 | 1.00 |
| P21180 | 1.87E+06 | 8.76E+05 | 1.24E+06 | 3.29E−04 | 8.98 | 18.40 | 2.05 |
| Q61233 | 6.48E+06 | 3.47E+06 | 3.27E+06 | 3.30E−04 | 0.13 | 1.58 | 12.59 |
| P01898 | 5.16E+07 | 4.34E+07 | 3.59E+07 | 4.34E−04 | 8.54 | 10.58 | 1.24 |
| P14847 | 1.19E+06 | 2.44E+06 | 9.75E+05 | 4.41E−04 | 9.17 | 18.43 | 2.01 |
| P14426 | 1.18E+06 | 7.56E+05 | 1.44E+06 | 4.65E−04 | 7.65 | 13.16 | 1.72 |
| Q61696 | 3.36E+07 | 6.05E+07 | 8.20E+07 | 4.76E−04 | 0.07 | 1.00 | 13.90 |
| P08226 | 2.07E+07 | 1.62E+07 | 1.93E+07 | 4.81E−04 | 2.82 | 2.91 | 1.03 |
| P01791 | 1.59E+06 | 1.90E+06 | 4.64E+06 | 5.89E−04 | 8.14 | 12.59 | 1.55 |
| P12246 | 7.53E+07 | 1.21E+08 | 7.22E+07 | 5.95E−04 | 6.21 | 7.40 | 1.19 |
| P01631 | 1.73E+08 | 2.74E+08 | 2.07E+08 | 6.83E−04 | 4.28 | 5.32 | 1.24 |
| P62806 | 2.84E+08 | 2.67E+08 | 3.77E+08 | 6.92E−04 | 0.02 | 0.12 | 5.68 |
| P01632 | 3.87E+07 | 6.18E+07 | 4.47E+07 | 7.03E−04 | 4.60 | 5.36 | 1.16 |
| P01844 | 5.59E+06 | 1.10E+07 | 6.37E+06 | 7.41E−04 | 6.67 | 11.54 | 1.73 |
| P01843 | 1.79E+07 | 3.08E+07 | 1.97E+07 | 7.77E−04 | 6.76 | 8.82 | 1.30 |
| O70362 | 2.28E+07 | 2.13E+07 | 1.34E+07 | 8.37E−04 | 5.12 | 10.31 | 2.01 |
| A6X935 | 2.10E+08 | 2.52E+08 | 2.22E+08 | 9.29E−04 | 4.46 | 4.64 | 1.04 |
| P28666 | 6.42E+07 | 5.60E+07 | 3.30E+07 | 9.30E−04 | 11.33 | 31.38 | 2.77 |
| P01630 | 1.52E+07 | 2.51E+07 | 1.65E+07 | 9.47E−04 | 6.11 | 7.66 | 1.25 |
| P01638 | 2.34E+07 | 3.65E+07 | 2.36E+07 | 1.12E−03 | 5.86 | 8.00 | 1.36 |
| Q923 D2 | 5.05E+05 | 7.57E+05 | 4.14E+05 | 1.13E−03 | 5.23 | 7.66 | 1.46 |
| P01635 | 9.16E+07 | 1.64E+08 | 1.20E+08 | 1.18E−03 | 4.57 | 5.69 | 1.24 |
| P01728 | 5.11E+06 | 5.97E+06 | 6.76E+06 | 1.20E−03 | 4.33 | 5.57 | 1.29 |
| P84750 | 7.69E+06 | 1.41E+07 | 1.14E+07 | 1.20E−03 | 5.74 | 7.36 | 1.28 |
| Q60994 | 1.58E+07 | 2.25E+07 | 1.61E+07 | 1.23E−03 | 6.32 | 7.01 | 1.11 |
| Q07968 | 2.29E+06 | 1.01E+06 | 1.49E+06 | 1.27E−03 | 10.28 | 20.39 | 1.98 |
| Q60590 | 2.22E+07 | 1.69E+07 | 5.14E+07 | 1.31E−03 | 6.76 | 10.08 | 1.49 |
| P09103 | 1.10E+07 | 7.18E+06 | 7.93E+06 | 1.32E−03 | 0.06 | 0.37 | 6.51 |
| P08607 | 4.15E+07 | 2.69E+07 | 2.94E+07 | 1.34E−03 | 4.71 | 9.09 | 1.93 |
| Q61702 | 3.45E+07 | 2.87E+07 | 1.12E+07 | 1.36E−03 | 7.04 | 16.36 | 2.32 |
| Q07456 | 3.59E+07 | 2.90E+07 | 1.60E+07 | 1.42E−03 | 6.30 | 12.40 | 1.97 |
| Q9JJN5 | 4.72E+06 | 5.74E+06 | 1.33E+07 | 1.48E−03 | 7.00 | 12.92 | 1.85 |
| Q80YC5 | 3.13E+06 | 3.06E+06 | 6.77E+06 | 1.49E−03 | 7.95 | 12.50 | 1.57 |
| P06909 | 2.20E+08 | 1.79E+08 | 1.02E+08 | 1.54E−03 | 8.11 | 19.96 | 2.46 |
| P01837 | 9.31E+08 | 1.28E+09 | 7.98E+08 | 1.56E−03 | 3.95 | 4.21 | 1.07 |
| P01636 | 4.32E+07 | 7.80E+07 | 5.27E+07 | 1.62E−03 | 5.02 | 6.50 | 1.29 |
| O08742 | 1.40E+06 | 6.22E+05 | 8.22E+05 | 1.64E−03 | 5.77 | 15.68 | 2.72 |
| P48678 | 1.37E+07 | 1.16E+07 | 2.93E+07 | 1.67E−03 | 0.03 | 0.01 | 0.38 |
| Q9DBD0 | 9.43E+07 | 3.47E+07 | 4.63E+07 | 1.69E−03 | 8.78 | 19.79 | 2.25 |
| Q921I1 | 2.52E+10 | 1.03E+10 | 1.39E+10 | 1.71E−03 | 5.37 | 11.00 | 2.05 |
| P29788 | 2.48E+07 | 2.32E+07 | 1.07E+07 | 1.76E−03 | 5.34 | 6.81 | 1.27 |
| Q61704 | 6.27E+07 | 6.19E+07 | 3.65E+07 | 1.86E−03 | 6.51 | 14.96 | 2.30 |
| P01642 | 6.50E+06 | 1.43E+07 | 7.59E+06 | 1.88E−03 | 5.63 | 8.17 | 1.45 |
| P28665 | 9.73E+08 | 8.51E+08 | 5.60E+08 | 1.92E−03 | 7.30 | 17.20 | 2.36 |
| P97350 | 3.88E+07 | 3.99E+07 | 5.59E+07 | 1.93E−03 | — | — | 18.40 |
| Q8VCG4 | 4.84E+06 | 5.57E+06 | 3.37E+06 | 1.97E−03 | 5.71 | 6.65 | 1.16 |
| Q8CG16 | 3.93E+06 | 4.45E+06 | 5.82E+06 | 2.07E−03 | 4.24 | 6.43 | 1.52 |
| Q61703 | 6.08E+07 | 5.31E+07 | 2.28E+07 | 2.20E−03 | 5.92 | 12.67 | 2.14 |
| Q61147 | 5.00E+08 | 4.20E+08 | 2.50E+08 | 2.33E−03 | 7.53 | 18.50 | 2.46 |
| Q61838 | 6.75E+09 | 6.26E+09 | 4.81E+09 | 2.58E−03 | 5.34 | 7.75 | 1.45 |
| P01807 | 5.65E+07 | 6.45E+07 | 1.45E+08 | 2.64E−03 | 6.46 | 8.08 | 1.25 |
| P00920 | 4.06E+06 | 7.33E+06 | 2.18E+06 | 2.92E−03 | 6.30 | 9.49 | 1.51 |
| P01746 | 1.05E+07 | 1.25E+07 | 3.63E+07 | 2.95E−03 | 6.97 | 9.42 | 1.35 |
| Q8BND5 | 1.95E+07 | 1.11E+07 | 7.00E+06 | 3.04E−03 | 5.32 | 6.21 | 1.17 |
| P23953 | 1.08E+09 | 4.46E+08 | 6.40E+08 | 3.19E−03 | 4.67 | 8.97 | 1.92 |
| P01723 | 1.11E+07 | 2.02E+07 | 9.29E+06 | 3.23E−03 | 4.48 | 5.96 | 1.33 |
| Q62351 | 9.34E+05 | 1.92E+06 | 1.57E+06 | 3.23E−03 | 9.36 | 15.12 | 1.62 |
| Q91Y97 | 4.41E+05 | 2.45E+05 | 1.85E+05 | 3.63E−03 | 5.99 | 7.15 | 1.19 |
| P20918 | 8.59E+07 | 1.66E+08 | 1.36E+08 | 3.68E−03 | 8.60 | 8.85 | 1.03 |
| O70456 | 1.37E+08 | 1.24E+08 | 1.57E+08 | 3.74E−03 | 0.10 | 0.23 | 2.26 |
| Q60930 | 4.48E+06 | 8.39E+06 | 1.36E+07 | 3.78E−03 | — | — | 14.83 |
| P01801 | 2.37E+07 | 2.75E+07 | 8.10E+07 | 3.91E−03 | 5.15 | 7.64 | 1.48 |
| P68372 | 3.60E+07 | 3.16E+07 | 5.98E+07 | 3.97E−03 | 0.08 | 0.30 | 3.64 |
| P01633 | 1.57E+08 | 2.23E+08 | 1.82E+08 | 3.97E−03 | 4.28 | 5.68 | 1.33 |
| O88947 | 6.71E+06 | 7.13E+06 | 2.26E+07 | 3.99E−03 | 4.83 | 10.10 | 2.09 |
| P32261 | 7.88E+07 | 9.30E+07 | 5.54E+07 | 4.11E−03 | 3.28 | 3.29 | 1.01 |
| P01660 | 1.58E+07 | 2.86E+07 | 1.28E+07 | 4.12E−03 | 4.24 | 5.78 | 1.36 |
| Q9ESB3 | 1.52E+08 | 1.39E+08 | 1.44E+08 | 4.21E−03 | 3.09 | 2.68 | 0.87 |
| P46412 | 4.04E+06 | 4.07E+06 | 4.74E+06 | 4.27E−03 | 4.86 | 4.29 | 0.88 |
| P26262 | 5.25E+06 | 4.61E+06 | 1.48E+07 | 4.29E−03 | 7.02 | 9.45 | 1.35 |
| Q8R121 | 2.50E+06 | 1.55E+06 | 2.85E+06 | 4.42E−03 | 7.89 | 7.06 | 0.90 |
| P04186 | 3.45E+07 | 5.28E+07 | 4.68E+07 | 4.42E−03 | 6.61 | 6.29 | 0.95 |
| P01027 | 4.43E+09 | 4.47E+09 | 2.03E+09 | 4.56E−03 | 3.75 | 4.10 | 1.09 |
| Q03734 | 9.52E+06 | 3.43E+06 | 7.33E+06 | 4.60E−03 | 4.84 | 4.26 | 0.88 |
| P01867 | 8.66E+08 | 9.39E+08 | 2.96E+09 | 4.77E−03 | 6.41 | 9.03 | 1.41 |
| P20152 | 7.30E+07 | 9.07E+07 | 5.15E+07 | 4.78E−03 | 0.31 | 0.15 | 0.50 |
| P01869 | 6.59E+08 | 7.58E+08 | 2.23E+09 | 4.79E−03 | 5.53 | 8.20 | 1.48 |
| P18527 | 4.31E+06 | 5.77E+06 | 1.67E+07 | 5.04E−03 | 6.07 | 8.96 | 1.48 |
| P01865 | 1.19E+09 | 1.33E+09 | 4.07E+09 | 5.06E−03 | 5.77 | 7.61 | 1.32 |
| P01864 | 4.67E+08 | 5.96E+08 | 1.79E+09 | 5.22E−03 | 5.84 | 7.51 | 1.29 |
| P18531 | 4.04E+06 | 4.59E+06 | 1.60E+07 | 5.40E−03 | 6.71 | 11.23 | 1.67 |
| P49182 | 2.69E+07 | 1.33E+07 | 8.21E+06 | 5.45E−03 | 5.45 | 9.21 | 1.69 |
| Q6GQT1 | 9.30E+05 | 6.32E+05 | 3.03E+05 | 5.56E−03 | 9.30 | 30.25 | 3.25 |
| Q64726 | 2.50E+07 | 1.11E+07 | 4.49E+07 | 5.82E−03 | 4.30 | 9.14 | 2.12 |
| Q8VCS0 | 5.70E+06 | 1.79E+06 | 1.85E+06 | 5.90E−03 | 5.34 | 12.67 | 2.37 |
| P06683 | 3.13E+06 | 4.14E+05 | 1.73E+06 | 6.07E−03 | 3.43 | 15.87 | 4.63 |
| P70274 | 4.34E+05 | 3.49E+05 | 1.71E+06 | 6.20E−03 | 6.37 | 15.71 | 2.47 |
| Q06890 | 3.45E+07 | 3.19E+07 | 3.09E+07 | 6.25E−03 | 3.06 | 3.09 | 1.01 |
| P17182 | 3.48E+07 | 1.08E+07 | 1.03E+07 | 6.78E−03 | 0.01 | 0.11 | 8.23 |
| P10126 | 9.09E+07 | 1.15E+08 | 1.57E+08 | 6.82E−03 | 0.09 | 0.13 | 1.49 |
| Q8CG14 | 2.87E+06 | 3.29E+06 | 2.16E+06 | 6.83E−03 | 6.90 | 8.55 | 1.24 |
| P07361 | 3.47E+06 | 2.30E+06 | 5.93E+06 | 6.97E−03 | 6.90 | 8.05 | 1.17 |
| P13020 | 4.52E+07 | 9.41E+07 | 9.19E+07 | 7.27E−03 | 6.77 | 6.81 | 1.01 |
| O08677 | 2.46E+08 | 2.30E+08 | 1.47E+08 | 7.48E−03 | 2.87 | 2.86 | 1.00 |
| Q61129 | 7.60E+06 | 1.60E+07 | 2.94E+07 | 7.58E−03 | 4.39 | 6.50 | 1.48 |
| O88968 | 3.17E+05 | Not | Not | 7.76E−03 | 2.63 | — | — |
| | | detected | detected | | | | |
| P02089 | 4.08E+06 | 3.38E+06 | 4.92E+06 | 7.80E−03 | 2.39 | 3.36 | 1.41 |
| P97290 | 3.77E+07 | 5.20E+07 | 5.19E+07 | 7.93E−03 | 5.38 | 5.71 | 1.06 |
| P01670 | 9.29E+06 | 2.39E+07 | 1.77E+07 | 8.13E−03 | 3.84 | 7.13 | 1.86 |
| Q02257 | 3.00E+07 | 3.27E+07 | 3.47E+07 | 8.33E−03 | 0.00 | 0.02 | 5.84 |
| Q01339 | 4.89E+07 | 7.75E+07 | 1.78E+08 | 8.38E−03 | 4.53 | 4.24 | 10.94 |
| P11859 | 2.54E+06 | 3.87E+06 | 1.38E+07 | 3.47E−03 | 5.74 | 8.08 | 1.41 |
| P21614 | 1.41E+08 | 2.01E+08 | 6.94E+08 | 9.66E−03 | 5.61 | 8.49 | 1.51 |
| P03987 | 1.34E+08 | 1.60E+08 | 5.95E+08 | 1.03E−02 | 5.35 | 7.63 | 1.43 |
| P97298 | 3.72E+06 | 4.41E+06 | 2.07E+07 | 1.04E−02 | 4.41 | 10.34 | 2.34 |
| P18524 | 3.11E+07 | 3.64E+07 | 1.23E+08 | 1.05E−02 | 4.45 | 6.54 | 1.47 |
| Q8VCM7 | 7.57E+06 | 7.34E+06 | 1.15E+07 | 1.07E−02 | 0.05 | 0.14 | 2.72 |
| P11276 | 8.26E+07 | 1.20E+08 | 2.16E+07 | 1.09E−02 | 10.30 | 19.88 | 1.93 |
| P07356 | 1.45E+08 | 1.40E+08 | 1.79E+08 | 1.09E−02 | 0.11 | 0.26 | 2.31 |
| Q9Z126 | 2.80E+07 | 2.65E+07 | 4.54E+07 | 1.20E−02 | 3.31 | 4.24 | 1.28 |
| Q00724 | 1.10E+07 | 1.10E+07 | 1.46E+07 | 1.31E−02 | 3.81 | 3.68 | 0.97 |
| Q8BTM8 | Not | 1.66E+05 | Not | 1.34E−02 | 5.37 | 20.46 | 3.81 |
| | detected | | detected | | | | |
| P42703 | 2.76E+06 | 2.15E+06 | 1.08E+06 | 1.44E−02 | 4.36 | 7.57 | 1.74 |
| P11247 | 1.34E+08 | 4.80E+07 | 5.98E+07 | 1.47E−02 | 0.03 | 0.44 | 13.33 |
| P11680 | 1.92E+06 | 3.68E+06 | 1.46E+07 | 1.63E−02 | 3.82 | 8.77 | 2.29 |
| P06330 | 2.79E+08 | 2.46E+08 | 9.08E+08 | 1.71E−02 | 4.90 | 6.30 | 1.28 |
| P70389 | 1.77E+06 | 4.58E+06 | 4.15E+06 | 1.72E−02 | 8.19 | 8.07 | 0.99 |
| P06336 | 1.30E+06 | 2.53E+06 | 2.52E+06 | 1.73E−02 | 7.04 | 8.73 | 1.24 |
| Q91X72 | 7.70E+09 | 5.26E+09 | 3.59E+09 | 1.74E−02 | 2.85 | 3.78 | 1.32 |
| Q03311 | 9.57E+04 | 2.07E+05 | 2.29E+05 | 1.96E−02 | 7.27 | 4.54 | 0.62 |
| Q9QWK4 | 1.15E+07 | 2.54E+07 | 7.32E+07 | 2.08E−02 | 4.32 | 5.76 | 1.33 |
| P07309 | 7.75E+08 | 6.31E+08 | 1.13E+09 | 2.16E−02 | 1.67 | 2.00 | 1.20 |
| Q06770 | 1.87E+07 | 1.82E+07 | 4.72E+06 | 2.24E−02 | 4.52 | 4.73 | 1.04 |
| P26039 | 1.31E+05 | 6.84E+04 | Not | 2.39E−02 | 20.60 | 37.86 | 1.84 |
| | | | detected | | | | |
| Q9D2Q8 | 2.54E+07 | 1.91E+07 | 2.21E+07 | 2.40E−02 | 0.02 | 0.07 | 2.71 |
| O89020 | 3.68E+07 | 4.16E+07 | 5.77E+07 | 2.53E−02 | 3.75 | 3.35 | 0.89 |
| Q9DBB9 | 4.52E+06 | 5.86E+06 | 7.92E+06 | 2.61E−02 | 5.31 | 4.42 | 0.83 |
| P10107 | 1.06E+09 | 1.20E+09 | 1.33E+09 | 2.74E−02 | 0.02 | 0.08 | 3.54 |
| P01749 | 3.23E+06 | 2.44E+06 | 1.78E+07 | 2.93E−02 | 4.48 | 9.06 | 2.02 |
| E9Q414 | 6.19E+07 | 5.79E+07 | 3.26E+07 | 2.94E−02 | 2.25 | 6.95 | 3.09 |
| P01887 | 1.08E+07 | 1.24E+07 | 2.00E+07 | 3.12E−02 | 2.75 | 3.45 | 1.25 |
| P09581 | 3.90E+04 | Not | 1.35E+05 | 3.15E−02 | 24.40 | 12.14 | 0.50 |
| | | detected | | | | | |
| P63017 | 1.82E+07 | 2.30E+07 | 2.06E+07 | 3.38E−02 | 0.17 | 0.21 | 1.30 |
| Q60963 | 2.18E+06 | 6.60E+05 | Not | 3.39E−02 | 3.50 | 7.35 | 2.10 |
| | | | detected | | | | |
| Q9JHH6 | 3.53E+06 | 2.65E+06 | 2.29E+05 | 3.44E−02 | 6.26 | 8.91 | 1.42 |
| Q08879 | 9.09E+04 | 1.25E+05 | 1.24E+05 | 3.75E−02 | 7.40 | 5.99 | 0.81 |
| P21107 | 2.57E+07 | 3.72E+07 | 1.51E+07 | 3.78E−02 | 0.43 | 0.06 | 0.13 |
| P07901 | 5.74E+06 | 1.05E+07 | 1.28E+07 | 3.83E−02 | — | — | 4.33 |
| P82198 | 1.34E+06 | 9.82E+05 | Not | 3.86E−02 | 3.54 | 4.06 | 1.14 |
| | | | detected | | | | |
| P07724 | 1.52E+11 | 1.42E+11 | 5.27E+10 | 3.90E−02 | 2.81 | 3.25 | 1.15 |
| P20029 | 8.69E+06 | 1.57E+07 | 5.68E+06 | 4.22E−02 | 0.41 | 0.15 | 0.37 |
| P11499 | 6.79E+06 | 1.44E+07 | 4.41E+06 | 4.25E−02 | 0.38 | 0.22 | 0.58 |
| P16301 | Not | Not | Not | 4.31E−02 | — | 3.00 | — |
| | detected | detected | detected | | | | |
| P31532 | 2.68E+07 | 2.88E+07 | 3.54E+07 | 4.42E−02 | 2.27 | 2.79 | 1.22 |
| Q01279 | 5.36E+06 | 7.04E+06 | 9.53E+06 | 4.61E−02 | 4.16 | 4.67 | 1.12 |
| P10605 | 9.86E+05 | 3.34E+05 | 2.96E+05 | 4.68E−02 | 6.30 | 4.28 | 0.68 |
| P17742 | 9.42E+07 | 8.65E+07 | 1.22E+08 | 4.70E−02 | 0.04 | 0.07 | 1.60 |
| P29699 | 1.26E+08 | 1.17E+08 | 3.51E+08 | 4.91E−02 | 2.11 | 2.80 | 1.33 |

Hierarchical clustering analysis of the 165 proteins exhibiting statistically significant changes among the three types of samples pulled down with beads, D-Cb, and L-Cb revealed that about 80% of the proteins pulled down with D-Cb showed increased abundance levels (>1.50-fold increases) compared to those pulled down with beads and L-Cb (FIG. 10). These 165 proteins, with a p-value <0.05 from ANOVA analysis, were further subjected to a Student's t-test analysis to identify proteins exhibiting statistically significant changes between those pulled down with D-Cb and L-Cb. Of the 165 proteins, 133 proteins showed statistically significant changes (p-value <0.05) between those pulled down with D-Cb and L-Cb. All 133 proteins exhibited increased protein abundance when pulled down with D-Cb compared with L-Cb (FIG. 11 and Table 5 show a hierarchical clustering analysis of the 133 proteins). Among these 133 proteins, 15 proteins were ligands of cell surface endothelial cell receptors known to mediate endothelial transcytosis for BBB penetration, such as transferrin receptor 1 (TfR1), lipoprotein receptor-related protein 1 (LRP1), and LRP2 (FIG. 9D and Table 6). ApoE, the previously known ligand for brain delivery via low-density lipoprotein receptor (LDLR), was also more abundantly found in the protein corona of D-Cb. A heat map showing the hierarchical clustering of these 15 proteins is presented in FIG. 9E.

To confirm that the brain distribution of D-Cb is based on TfR1, LRP1, LRP2, and LDLR-mediated endothelial transcytosis, the inventors investigated the uptake of D-Cb with a protein corona into mouse endothelial cells (bEnd.3) after inhibiting the major receptors involved in the transport of various ligands across the BBB (FIG. 9F). In the presence of serum, the cellular uptake level of D-Cb was significantly inhibited by antibodies against these receptors. However, in the absence of serum, the cellular uptake efficiency of D-Cb was not significantly affected by antibodies against TfR1, LRP1, LRP2, and LDLR. The uptake level of D-Cb was reduced by 80% with TfR1 Ab, while the uptake level of D-Cb was inhibited by less than 30% with LRP1 Ab. The uptake level was moderately decreased by both LRP2 Ab and LDLR Ab. These results suggest that the BBB penetration of D-Cb is mainly mediated by TfR1 through interaction with transferrin in the protein corona, but all receptors investigated play an important role in transcytosis. Similarly, transcytosis of D-Cb through an in vitro BBB monolayer was also significantly reduced by the antibodies in the presence of serum (FIGS. 9G and 9A), but the effect of antibodies on transcytosis of D-Cb in the monolayer model was negligible in the absence of serum. These results indicate that endothelial transcytosis of D-Cb for BBB penetration depends on a protein corona containing ligands for endothelial receptors. In contrast, the cellular uptake efficiency of L-Cb in the endothelial cells was not affected by the presence of antibodies, regardless of the serum (FIG. 12B). This suggests that the protein corona-assisted transcytosis of L-Cb is unlikely due to its low protein adsorption property (FIG. 9B). The tight junctions were not disrupted by the treatment of antibodies and Cbs, as assessed by TEER levels (FIG. 12C).

TABLE 6

| | | | P-value | Abundance | Ligands |
|---|---|---|---|---|---|
| Accession | | Gene | (Student's | ratio (D-Cb/ | of LRP1 and |
| No. | Protein description | symbol | t-test) | L-Cb)^{a} | LRP2^{b} |
| P16301 | Phosphatidylcholine-sterol | LCAT | 5.58E−03 | — | |
| | acyltransferase | | | | |
| P09581 | Macrophagecolony- | CSF1R | 2.72E−02 | 24.40 | |
| | stimulating factor 1 receptor | | | | |
| P06684 | Complement C5 | C5 | 1.86E−04 | 11.65 | |
| P28666 | Murinoglobulin-2 | MUG2 | 6.56E−03 | 11.33 | |
| O88783 | Coagulation factor V | F5 | 2.20E−03 | 10.34 | |
| P11276 | Fibronectin | FN1 | 1.49E−02 | 10.30 | |
| Q07968 | Coagulation factor XIII B | F13B | 1.48E−03 | 10.28 | |
| | chain | | | | |
| P35441 | Thrombospondin-1 | THBS1 | 2.09E−03 | 9.45 | |
| Q62351 | Transferrin receptor protein 1 | TFRC | 6.51E−03 | 9.36 | |
| Q6GQT1 | Alpha-2-macroglobulin-P | A2M | 1.10E−02 | 9.30 | |
| | (Alpha-2-macroglobulin) | | | | |
| P14847 | C-reactive protein | CRP | 1.93E−03 | 9.17 | |
| P98086 | Complement C1q | C1QA | 5.58E−04 | 8.99 | |
| | subcomponent subunit A | | | | |
| P21180 | Complement C2 | C2 | 3.86E−04 | 8.98 | |
| Q9DBD0 | Inhibitor of carbonic | ICA | 3.46E−03 | 8.78 | |
| | anhydrase | | | | |
| P20918 | Plasminogen | PLG | 5.04E−03 | 8.60 | O |
| P01898 | H-2 class I | H2-Q10 | 2.72E−04 | 8.54 | |
| | histocompatibility | | | | |
| | antigen, Q10 alpha chain | | | | |
| Q9R098 | Hepatocyte growth factor activator | HGFAC | 1.46E−04 | 8.31 | |
| P70389 | Insulin-like growth factor- | IGFALS | 8.12E−03 | 8.19 | |
| | binding protein complex acid | | | | |
| | labile subunit | | | | |
| P01791 | Ig heavy chain V region HPCM6 | | 2.14E−03 | 8.14 | |
| P06909 | Complement factor H | CFH | 8.10E−03 | 8.11 | |
| Q80YC5 | Coagulation factor XII | F12 | 1.03E−03 | 7.95 | |
| Q8R121 | Protein Z-dependent protease inhibitor | SERPINA10 | 4.66E−04 | 7.89 | |
| P14426 | H-2 class I | H2-D1 | 6.00E−04 | 7.65 | |
| | histocompatibility | | | | |
| | antigen, D-K alpha chain | | | | |
| Q61147 | Ceruloplasmin | CP | 1.19E−02 | 7.53 | |
| P01872 | Immunoglobulin heavy | IGHM | 1.74E−03 | 7.42 | |
| | constant mu | | | | |
| Q08879 | Fibulin-1 | FBLN1 | 2.10E−02 | 7.40 | |
| P52430 | Serum | PON1 | 2.81E−04 | 7.33 | |
| | paraoxonase/arylesterase 1 | | | | |
| P28665 | Murinoglobulin-1 | MUG1 | 1.10E−02 | 7.30 | |
| P14106 | Complement C1q | C1QB | 1.21E−03 | 7.28 | |
| | subcomponent subunit B | | | | |
| Q03311 | Cholinesterase | BCHE | 9.38E−03 | 7.27 | |
| P01029 | Complement C4-B | C4B | 7.78E−04 | 7.25 | |
| Q02105 | Complement C 1 q | C1QC | 4.81E−04 | 7.11 | |
| | subcomponent subunit C | | | | |
| Q61702 | Inter-alpha-trypsin inhibitor | ITIH1 | 3.20E−03 | 7.04 | |
| | heavy chain H1 | | | | |
| P06336 | Ig epsilon chain C region | | 8.79E−03 | 7.04 | |
| P26262 | Plasma kallikrein | KLKB1 | 5.00E−03 | 7.02 | |
| Q9JJN5 | Carboxypeptidase N catalytic chain | CPN1 | 1.91E−03 | 7.00 | |
| P01746 | Ig heavy chain V region 93G7 | | 5.04E−03 | 6.97 | |
| P07361 | Alpha-1-acid glycoprotein 2 | ORM2 | 1.03E−03 | 6.90 | |
| Q8CG14 | Complement C1s-1 subcomponent | C1S1 | 5.40E−03 | 6.90 | |
| P13020 | Gelsolin | GSN | 7.95E−03 | 6.77 | O |
| Q60590 | Alpha-1-acid glycoprotein 1 | ORM1 | 3.38E−03 | 6.76 | |
| P01843 | Ig lambda-1 chain C region | | 1.42E−03 | 6.76 | |
| P18531 | Ig heavy chain V region 3-6 | IGHV3-6 | 7.65E−03 | 6.71 | |
| P01844 | Ig lambda-2 chain C region | IGLC2 | 8.08E−04 | 6.67 | |
| P04186 | Complement factor B | CFB | 4.49E−03 | 6.61 | |
| Q61704 | Inter-alpha-trypsin inhibitor | ITIH3 | 9.33E−03 | 6.51 | |
| | heavy chain H3 | | | | |
| P01807 | Ig heavy chain V region X44 | | 3.92E−03 | 6.46 | |
| P01867 | Immunoglobulin heavy | IGHG2B | 5.93E−03 | 6.41 | |
| | constant gamma 2B | | | | |
| Q9Z1R3 | Apolipoprotein M | APOM | 2.26E−04 | 6.38 | O |
| P70274 | Selenoprotein P | SELENOP | 1.64E−02 | 6.37 | O |
| Q60994 | Adiponectin | ADIPOQ | 4.69E−04 | 6.32 | |
| Q07456 | Protein AMBP | AMBP | 5.27E−03 | 6.30 | |
| P00920 | Carbonic anhydrase 2 | CA2 | 6.46E−03 | 6.30 | |
| P10605 | Cathepsin B | CTSB | 3.19E−02 | 6.30 | O |
| Q9JHH6 | Carboxypeptidase B2 | CPB2 | 2.75E−02 | 6.26 | |
| P12246 | Serum amyloid P-component | APCS | 1.48E−03 | 6.21 | |
| P01630 | Ig kappa chain V-II region 7S34.1 | | 1.34E−03 | 6.11 | |
| P18527 | Ig heavy chain V region 914 | | 6.62E−03 | 6.07 | |
| Q91Y97 | Fructose-bisphosphate | ALDOB | 2.55E−03 | 5.99 | |
| | aldolase B | | | | |
| Q61703 | Inter-alpha-trypsin inhibitor | ITIH2 | 9.25E−03 | 5.92 | |
| | heavy chain H2 | | | | |
| P01638 | Ig kappa chain V-V region L6 | | 1.62E−03 | 5.86 | |
| | (Fragment) | | | | |
| P01864 | Lg gamma-2A chain C | | 5.93E−03 | 5.84 | |
| | region | | | | |
| | secreted form | | | | |
| O08742 | Platelet glycoprotein V | GP5 | 3.92E−03 | 5.77 | |
| P01865 | Ig gamma-2A chain C region, | IGH-1A | 6.41E−03 | 5.77 | |
| | membrane-bound form | | | | |
| P11859 | Angiotensinogen | AGT | 8.73E−03 | 5.74 | |
| P84750 | Ig kappa chain V region Mem5 | | 1.23E−03 | 5.74 | |
| | (Fragment) | | | | |
| Q8VCG4 | Complement component C8 | C8G | 1.69E−03 | 5.71 | |
| | gamma chain | | | | |
| P01642 | Ig kappa chain V-V region L7 | GM10881 | 2.42E−03 | 5.63 | |
| | (Fragment) | | | | |
| P21614 | Vitamin D-binding protein | GC | 1.09E−02 | 5.61 | O |
| P01869 | Ig gamma-1 chain C | IGHG1 | 6.82E−03 | 5.53 | |
| | region, | | | | |
| | membrane-bound form | | | | |
| P49182 | Heparin cofactor 2 | SERPIND 1 | 4.13E−03 | 5.45 | |
| P97290 | Plasma protease C1 inhibitor | SERPING1 | 4.09E−03 | 5.38 | |
| Q921I1 | Serotransferrin | TF | 5.41E−03 | 5.37 | |
| P03987 | Ig gamma-3 chain C region | | 1.01E−02 | 5.35 | |
| P29788 | Vitronectin | VTN | 2.03E−03 | 5.34 | |
| Q8VCS0 | N-acetylmuramoyl-L-alanine amidase | PGLYRP2 | 9.14E−03 | 5.34 | |
| Q61838 | Pregnancy zone protein | PZP | 5.39E−03 | 5.34 | |
| Q8BND5 | Sulfhydryl oxidase 1 | QSOX1 | 2.14E−03 | 5.32 | |
| Q9DBB9 | Carboxypeptidase N subunit 2 | CPN2 | 3.77E−03 | 5.31 | |
| P19221 | Prothrombin | F2 | 1.25E−05 | 5.29 | |
| P01878 | Ig alpha chain C region | | 4.99E−04 | 5.29 | |
| Q923D2 | Flavin reductase (NADPH) | BLVRB | 1.20E−03 | 5.23 | |
| P01801 | Ig heavy chain V-III region J606 | | 7.23E−03 | 5.15 | |
| O70362 | Phosphatidylinositol-glycan- | GPLD1 | 5.41E−03 | 5.12 | |
| | specific phospholipase D | | | | |
| P01636 | Ig kappa chain | | 2.20E−03 | 5.02 | |
| | V-V region MOPC 149 | | | | |
| P06330 | Ig heavy chain V region AC38 | | 1.59E−02 | 4.90 | |
| | 205.12 | | | | |
| P46412 | Glutathione peroxidase 3 | GPX3 | 5.25E−04 | 4.86 | |
| Q03734 | Serine protease inhibitor A3M | SERPINA3 M | 5.35E−03 | 4.84 | |
| O88947 | Coagulation factor X | F10 | 1.25E−02 | 4.83 | |
| P08607 | C4b-binding protein | C4BPA | 4.03E−03 | 4.71 | |
| P23953 | Carboxylesterase 1C | CES1C | 8.38E−03 | 4.67 | |
| P01632 | Ig kappa chain V-I region S107A | IGKV7-33 | 1.09E−03 | 4.60 | |
| P01635 | Immunoglobulin kappa | IGKV12-41 | 1.72E−03 | 4.57 | |
| | chain variable 12-41 (Fragment) | | | | |
| Q01339 | Beta-2-glycoprotein 1 | APOH | 8.03E−03 | 4.53 | O |
| Q06770 | Corticosteroid-binding globulin | SERPINA6 | 1.38E−02 | 4.52 | |
| P01723 | Ig lambda-1 chain V region | | 3.82E−03 | 4.48 | |
| P01749 | Ig heavy chain V region 3 | IGHV1-61 | 2.86E−02 | 4.48 | |
| A6X935 | Inter alpha-trypsin | ITIH4 | 3.21E−04 | 4.46 | |
| | inhibitor, heavy chain 4 | | | | |
| P18524 | Ig heavy chain V region RF | | 1.27E−02 | 4.45 | |
| P97298 | Pigment epithelium-derived factor | SERPINF1 | 1.98E−02 | 4.41 | |
| Q61129 | Complement factor I | CFI | 1.36E−02 | 4.39 | |
| P42703 | Leukemia inhibitory factor receptor | LIFR | 2.54E−02 | 4.36 | |
| Q61247 | Alpha-2-antiplasmin | SERPINF2 | 8.72E−06 | 4.35 | |
| P01728 | Ig lambda-2 chain V region | | 1.29E−03 | 4.33 | |
| Q9QWK4 | CD5 antigen-like | CD5L | 1.82E−02 | 4.32 | |
| Q64726 | Zinc-alpha-2-glycoprotein | AZGP1 | 1.22E−02 | 4.30 | |
| P01633 | Immunoglobulin kappa | IGKV6-17 | 4.61E−03 | 4.28 | |
| | chain variable 6-17 | | | | |
| P01631 | Ig kappa chain V-II region 26- 10 | | 1.23E−03 | 4.28 | |
| Q8CG16 | Complement C1r-A subcomponent | C1RA | 1.98E−03 | 4.24 | |
| P01660 | Ig kappa chain V-III region PC | | 4.43E−03 | 4.24 | |
| | 3741/TEPC 111 | | | | |
| Q01279 | Epidermal growth factor receptor | EGFR | 2.15E−02 | 4.16 | |
| P01837 | Immunoglobulin kappa constant | IGKC | 7.19E−04 | 3.95 | |
| P01670 | Ig kappa chain V-III region PC 6684 | | 7.70E−03 | 3.84 | |
| P11680 | Properdin | CFP | 2.82E−02 | 3.82 | |
| Q00724 | Retinol-binding protein 4 | RBP4 | 1.09E−03 | 3.81 | O |
| P01027 | Complement C3 | C3 | 3.81E−03 | 3.75 | |
| O89020 | Afamin | AFM | 2.24E−03 | 3.75 | |
| Q61646 | Haptoglobin | HP | 4.01E−04 | 3.66 | |
| P82198 | Transforming growth | TGFBI | 3.41E−03 | 3.54 | |
| | factor-beta-induced protein ig-h3 | | | | |
| Q60963 | Platelet-activating factor | PLA2G7 | 1.76E−02 | 3.50 | |
| | acetylhydrolase | | | | |
| P06683 | Complement component C9 | C9 | 3.56E−02 | 3.43 | |
| Q9Z126 | Platelet factor 4 | PF4 | 1.77E−02 | 3.31 | |
| P32261 | Antithrombin-III | SERPINC1 | 5.64E−03 | 3.28 | |
| Q9ESB3 | Histidine-rich glycoprotein | HRG | 1.29E−03 | 3.09 | |
| Q06890 | Clusterin (Apolipoprotein J) | CLU | 3.25E−03 | 3.06 | O |
| O08677 | Kininogen-1 | KNG1 | 2.39E−03 | 2.87 | |
| Q91X72 | Hemopexin | HPX | 1.35E−02 | 2.85 | |
| P08226 | Apolipoprotein E | APOE | 9.95E−04 | 2.82 | O |
| P07724 | Albumin | ALB | 2.15E−02 | 2.81 | O |
| P01887 | Beta-2-microglobulin | B2M | 2.45E−02 | 2.75 | O |
| P02089 | Hemoglobin subunit beta-2 | HBB-B2 | 7.91E−03 | 2.39 | O |
| P31532 | Serum amyloid A-4 protein | SAA4 | 2.68E−02 | 2.27 | |
| P07309 | Transthyretin | TTR | 3.23E−02 | 1.67 | O |

### 4. D-Cb as a Drug Delivery System for Targeting GBM

After observing the possibility of a protein corona on D-Cb penetrating the BBB, the inventors hypothesized that D-Cb could serve as a platform for targeting glial cells in the brain when coated with a protein corona containing ligands of endocytic receptors, as these receptors are also abundantly expressed in GBM cells such as U87MG (FIG. 13A). To investigate the possibility of targeting glioblastoma in vivo, the inventors injected D-Cb intravenously into mice bearing orthotopic GBM, which was generated by intracranial injection of firefly luciferase (FLuc)- and green fluorescence protein (GFP)-expressing U87MG cells (U87MG-FLuc-GFP), and analyzed its distribution in the brain tissue. Representative ex vivo imaging of the brain at 2 hours post injection revealed a clear distribution of D-Cb in the brain of GBM-bearing mice (FIG. 13B). The brain distribution level of D-Cb in GBM-bearing mice was about 0.42% ID/g, as quantified by measuring the intensity of Cy5.5 in brain lysates, and 0.43% ID/g, as quantified by PAGE analysis to estimate the amount of intact form of the structure. These levels were higher than the brain distribution level of D-Cb in healthy mice (FIG. 13C and FIG. 14). Confocal fluorescence microscopic images of brain sections showed D-Cb accumulated in the tumor region (FIG. 13D). The enhanced brain distribution and tumor accumulation of D-Cb may be due to the endothelial cell receptors expressed in U87MG cells as well as in the endothelial cells, which interact with the ligands of the protein corona on D-Cb. Indeed, cellular uptake of D-Cb into U87MG cells was significantly inhibited by antibodies against these receptors in the presence of serum (FIG. 13E), which may promote the formation of a protein corona on D-Cb. However, the uptake level was not significantly affected by the antibodies in the absence of serum. The internalization of D-Cb into U87MG cells after transcytosis through endothelial cells was examined using an in vitro blood-tumor barrier (BTB) model (FIG. 13F). D-Cb added in the apical side were transcytosed through endothelial cells (bEnd.3) to reach the basolateral side and subsequently internalized into U87MG cells (FIG. 13G). These results support the findings related to in vivo tumor distribution of D-Cb in the brain.

Next, L-Cb was intravenously injected into glioblastoma-induced mice for comparison. Neither significant brain distribution nor accumulation of L-Cb in the tumor was observed (FIGS. 13B-D). The cellular uptake efficiency of L-Cb into U87MG cells was not affected by the presence of antibodies, regardless of the serum (FIG. 15). These results are consistent with the cell uptake pattern observed in bEnd.3 cells (FIG. 4F). In the in vitro BTB model, the internalization of L-Cb into U87MG cells was found to be approximately 3-fold lower than that of D-Cb (FIGS. 13G and 13H). This may be attributed to the lack of L-Cb protein corona necessary for interaction with the endocytic receptors, which may limit its penetration through the BBB and BTB before reaching U87MG cells and limit its uptake into the glioblastoma cells even after penetration. Overall, these results suggest that the ligands of the endocytic receptors in protein corona are responsible for the accumulation of D-Cb in U87MG tumors in the brain.

### 5. Systemic Brain Delivery of ASO Using D-Cb for Treatment of GBM

After confirming that the protein corona recognizable by receptors mediating BBB penetration is a key factor in the brain distribution of D-Cb and its accumulation in glioblastoma tumors, the inventors investigated the potential of D-Cb as a system for delivering ASO to the brain for the treatment of glioblastoma. As a model therapeutic ASO, an ASO sequence targeting polo-like kinase 1 (PLK1) mRNA, a potential target for the treatment of glioblastoma, was selected.

ASO was loaded onto D-Cb by adding the ASO sequence to the 3′-end of the S6 strand (FIG. 16A, FIG. 17 and Table 7). The hydrodynamic size of ASO@D-Cb was 8.32±0.19 nm, slightly larger than that of D-Cb (FIG. 18). The gene silencing effect of ASO@D-Cb was initially evaluated in glioblastoma cells (U87MG), where D-Cb enhanced cellular uptake of ASO by approximately 10-fold, as estimated by fluorescence microscopy and flow cytometry (FIGS. 16B and 16C). Treatment with ASO@D-Cb reduced PLK1 mRNA in U87MG by approximately 50% compared to phosphate buffered saline (PBS)-treated cells (negative control), as determined by quantitative reverse-transcriptase PCR (qRT-PCR) (FIG. 16D). The gene silencing effect by ASO@D-Cb was comparable to that of the positive control, lipofectamine-delivered ASO. D-Cb loaded with a scrambled ASO sequence (ASO-SC@D-Cb) did not exhibit significant target gene silencing activity, indicating that gene silencing by ASO@D-Cb is achieved in a target sequence-specific manner. The downregulation of PLK1 mRNA subsequently led to PLK1 protein level in the cells, as observed by western blot analysis (FIGS. 16D and 19).

TABLE 7

| | | SEQ |
|---|---|---|
| | | ID |
| ASO | Sequence | NO. |
| S6-T5-ASO | CTT AAT GAC TTT GGC CGG | 20 |
| (PLK1) | CGC TTT GAC CTT CTG CTT | |
| | ATG | |
| | TCC CCT A TTT TTC* A*TT | |
| | AAG CAG CT*C* G | |
| S6-T5-ASO | CTT AAT GAC TTT GGC CGG | 21 |
| (SC) | CGC TTT GAC CTT CTG CTT | |
| | ATG | |
| | TCC CCT A TTT TTC* A*GG | |
| | GCTGACAG*C* G | |
| ASO(Ctrl) | C* A*TT AAG CAG CT*C* G | 22 |

After confirming the cellular activity of ASO@D-Cb, Cy5.5-labeled ASO@D-Cb was intravenously injected into orthotopic GBM-induced mice, and the fluorescence intensity in the head regions of the mice was monitored (FIG. 16E). Both ASO and ASO@D-Cb were detected in the head region 10 minutes after injection. After 1-2 hours, the Cy5.5 intensity in the head region peaked and then slowly decreased. The brain was harvested and imaged using the Cy5.5 label on ASO at 2 and 24 hours post injection (FIGS. 16F and 20). ASO@D-Cb showed a higher brain distribution level than ASO alone. PAGE analysis of brain lysates revealed that ASO delivered by D-Cb was mostly in intact form (FIGS. 16G, 21A and 21B), whereas no intact ASO was detected when the ASO was delivered without D-Cb, indicating that ASO alone was unable to reach the brain. The brain distribution of ASO@D-Cb in glioblastoma mice was not significantly different from that in healthy BALB/c mice (FIGS. 16G, 22, 23A and 23B). Fluorescence microscopy examination of brain sections showed that the majority of ASO@D-Cb was distributed within the U87MG-FLuc-GFP tumor region, which was indicated by the green fluorescence signals due to GFP expression (FIGS. 16H and 16I). ASO uptake in the brain tissue section was negligible. This indicates that tumor distribution of D-Cb was not significantly affected by the loading of ASO.

### 6. Therapeutic Efficacy of Systemically Administered ASO@D-Cb for GBM Treatment

To evaluate the in vivo therapeutic efficacy of ASO@D-Cb, mice were serially administered with 5 doses at 2-day intervals (FIGS. 24 and 24B). By generating a glioblastoma model using U87MG-FLuc-GFP cells, it was possible to monitor tumor size in vivo using luminescence (FIG. 24C). Tumor growth in mice treated with ASO@D-Cb was inhibited by about 85% compared to tumor growth in untreated control mice (FIG. 24D). In contrast, free ASO failed to inhibit tumor growth, demonstrating the need for delivery with D-Cb to achieve significant therapeutic potency for glioblastoma. Treatment with ASO-SC@D-Cb did not exhibit significant potency for glioblastoma treatment, indicating that the potency of ASO@D-Cb resulted from ASO sequences specific to the target mRNA. Hematoxylin and eosin (H&E) staining of tumor tissue sections revealed damaged areas only after treatment with ASO@D-Cb (FIG. 24E). No significant tissue damage was observed in other major organs (FIG. 25). Apoptotic cell death induced by ASO@D-Cb in the brain was also confirmed by a transferase-mediated nick end labeling (TUNEL) assay (FIG. 24E). Accordingly, PLK1 mRNA and protein levels in tumor lysates were downregulated only by ASO@D-Cb, as determined by qRT-PCR and western blot analysis, respectively (FIGS. 24F and 26). These results suggest that the antitumor effect of ASO@D-Cb was mediated by apoptotic cell death resulting from PLK1 downregulation. This therapeutic potency of ASO@D-Cb overall alleviated the weight loss of mice caused by the glioblastoma toxicity (FIG. 27).

While D-Cb significantly enhances the systemic brain delivery of ASO for the treatment of glioblastoma, there is still room for improvement in the brain distribution level of D-Cb. The protein corona on D-Cb, which appears to contain proteins contributing to BBB penetration, may be further optimized.

Although a number of embodiments have been described with reference to limited drawings, one of ordinary skill in the art will recognize that various modifications and alterations may be made to these embodiments based on the above detailed description. For example, suitable results may be achieved if the described techniques are performed in a different order, and/or if components in a described system, architecture, device, or circuit are combined in a different manner, and/or replaced or supplemented by other components or their equivalents.

Therefore, other implementations, other examples, and equivalents to the claims are also within the scope of the following claims.

What is claimed is:

1. A drug delivery system for penetrating the blood-brain barrier (BBB), consisting of deoxyribonucleic acid (DNA),
wherein the drug delivery system has a double-stranded DNA as a frame,
wherein the drug delivery system forms a cube structure.

2. The drug delivery system of claim 1, wherein the DNA is D-form DNA.

3. The drug delivery system of claim 1, wherein one side of the cube structure has a length of greater than 5 bp and less than 30 bp.

4. The drug delivery system of claim 1, wherein a protein corona is formed on a surface of the drug delivery system, and the drug delivery system passes through the BBB via transcytosis.

5. The drug delivery system of claim 1, wherein the drug delivery system is for loading drugs for the treatment of brain tumors.

6. The drug delivery system of claim 5, wherein the brain tumor is glioblastoma (GBM).

7. A pharmaceutical composition for treating brain tumors, comprising DNA nanostructures loaded with drugs as active ingredients,
wherein the DNA nanostructures form a cube structure using double-stranded DNA as a frame.

8. The pharmaceutical composition of claim 7, wherein the DNA is D-form DNA.

9. The pharmaceutical composition of claim 7, wherein one side of the cube structure has a length of greater than 5 bp and less than 30 bp.

10. The pharmaceutical composition of claim 7, wherein the drug is an oligonucleotide or a hydrophobic drug.

11. The pharmaceutical composition of claim 7, wherein the brain tumor is glioblastoma.


SEQUENCE LISTING (22 sequences)

SEQ ID NO: 1 - DNA, 34 bp, synthetic construct
  misc_feature 1..34: note = Td_S1
  source 1..34: mol_type = other DNA
gggatcccgattcgagacagcatttctcccacac

SEQ ID NO: 2 - DNA, 34 bp, synthetic construct
  misc_feature 1..34: note = Td_S2
  source 1..34: mol_type = other DNA
cgtggtaggttttgctgtctcgttagcgccggcc

SEQ ID NO: 3 - DNA, 34 bp, synthetic construct
  misc_feature 1..34: note = Td_S3
  source 1..34: mol_type = other DNA
tcgggatcccttcacgggcaacttggccggcgct

SEQ ID NO: 4 - DNA, 34 bp, synthetic construct
  misc_feature 1..34: note = Td_S4
  source 1..34: mol_type = other DNA
acctaccacgttgttgcccgtgttgtgtgggaga

SEQ ID NO: 5 - DNA, 34 bp, synthetic construct
  misc_feature 1..34: note = Tp_S1
  source 1..34: mol_type = other DNA
tgctgtctcgttcgtggtaggtttgcagaaggtc

SEQ ID NO: 6 - DNA, 46 bp, synthetic construct
  misc_feature 1..46: note = Tp_S2
  source 1..46: mol_type = other DNA
cgagacagcattcacgggcaactttctcccacacttgggaaaggtc

SEQ ID NO: 7 - DNA, 34 bp, synthetic construct
  misc_feature 1..34: note = Tp_S3
  source 1..34: mol_type = other DNA
gtgtgggagattccgagggatcttgcggattgta

SEQ ID NO: 8 - DNA, 46 bp, synthetic construct
  misc_feature 1..46: note = Tp_S4
  source 1..46: mol_type = other DNA
ggccggcgctttacctaccacgttgacctttccctttacaatccgc

SEQ ID NO: 9 - DNA, 46 bp, synthetic construct
  misc_feature 1..46: note = Tp_S5
  source 1..46: mol_type = other DNA
agcgccggccttgatccctcggttgttgcccgtgttgaccttctgc

SEQ ID NO: 10 - DNA, 46 bp, synthetic construct
  misc_feature 1..46: note = Cb_S1
  source 1..46: mol_type = other DNA
gtgtgggagattagtcattaagtttacaatccgcttgtaatcgtag

SEQ ID NO: 11 - DNA, 46 bp, synthetic construct
  misc_feature 1..46: note = Cb_S2
  source 1..46: mol_type = other DNA
gttgcccgtgttctacgattacttggtcgggaaattcgtggtaggt

SEQ ID NO: 12 - DNA, 46 bp, synthetic construct
  misc_feature 1..46: note = Cb_S3
  source 1..46: mol_type = other DNA
gcggattgtatttaggggacatttcgagacagcatttttcccgacc

SEQ ID NO: 13 - DNA, 46 bp, synthetic construct
  misc_feature 1..46: note = Cb_S4
  source 1..46: mol_type = other DNA
gcagaaggtcttccgagggatcttacctaccacgtttgctgtctcg

SEQ ID NO: 14 - DNA, 46 bp, synthetic construct
  misc_feature 1..46: note = Cb_S5
  source 1..46: mol_type = other DNA
agcgccggcctttctcccacacttcacgggcaacttgatccctcgg

SEQ ID NO: 15 - DNA, 46 bp, synthetic construct
  misc_feature 1..46: note = Cb_S6
  source 1..46: mol_type = other DNA
cttaatgactttggccggcgctttgaccttctgcttatgtccccta

SEQ ID NO: 16 - DNA, 22 bp, synthetic construct
  misc_feature 1..22: note = PLK1_Forward
  source 1..22: mol_type = other DNA
gcacagtgtcaatgcctccaag

SEQ ID NO: 17 - DNA, 22 bp, synthetic construct
  misc_feature 1..22: note = PLK1_Reverse
  source 1..22: mol_type = other DNA
gccgtacttgtccgaatagtcc

SEQ ID NO: 18 - DNA, 22 bp, synthetic construct
  misc_feature 1..22: note = GAPDH_Forward
  source 1..22: mol_type = other DNA
gtctcctctgacttcaacagcg

SEQ ID NO: 19 - DNA, 22 bp, synthetic construct
  misc_feature 1..22: note = GAPDH_Reverse
  source 1..22: mol_type = other DNA
accaccctgttgctgtagccaa

SEQ ID NO: 20 - DNA, 65 bp, synthetic construct
  misc_feature 1..65: note = S6-T5-ASO(PLK1)
  source 1..65: mol_type = other DNA
cttaatgactttggccggcgctttgaccttctgcttatgtcccctatttttcattaagcagctcg

SEQ ID NO: 21 - DNA, 65 bp, synthetic construct
  misc_feature 1..65: note = S6-T5-ASO(SC)
  source 1..65: mol_type = other DNA
cttaatgactttggccggcgctttgaccttctgcttatgtcccctatttttcagggctgacagcg

SEQ ID NO: 22 - DNA, 14 bp, synthetic construct
  misc_feature 1..14: note = ASO(Ctrl)
  source 1..14: mol_type = other DNA
cattaagcagctcg